(19) United States
(12) Patent Application Publication
(10) Pub. No.: US 2024/0158806 A1
(43) Pub. Date: May 16, 2024

(54) BACTERIAL STRAINS WITH TOXIN COMPLEX FOR INSECT CONTROL

(71) Applicant: PIONEER HI-BRED INTERNATIONAL, INC., JOHNSTON, IA (US)

(72) Inventors: BRIAN J. GIBBONS, REDWOOD CITY, CA (US); STEVEN D. GRUVER, PACIFICA, CA (US); LU LIU, PALO ALTO, CA (US); YOUNG-JUN PARK, FREMONT, CA (US); JUN-ZHI WEI, JOHNSTON, IA (US); GENHAI ZHU, SAN JOSE, CA (US)

(73) Assignee: PIONEER HI-BRED INTERNATIONAL, INC., JOHNSTON, IA (US)

(21) Appl. No.: 18/394,021

(22) Filed: Dec. 22, 2023

Related U.S. Application Data
(62) Division of application No. 17/457,070, filed on Dec. 1, 2021, now Pat. No. 11,891,613.
(60) Provisional application No. 63/126,645, filed on Dec. 17, 2020.

(51) Int. Cl. C12N 15/82; A01N 63/20

(52) U.S. Cl. CPC C12N 15/8286; A01N 63/20

(57) ABSTRACT

Biological strains, compositions, and methods of using the strains and compositions for reducing overall insect damage.

### FIELD

Biological strains, compositions, and methods of using the strains and compositions for reducing overall insect damage. Also provided are novel genes that encode pesticidal proteins. These pesticidal proteins and the nucleic acid sequences that encode them are useful in preparing pesticidal formulations and in the production of transgenic pest-resistant plants.

### REFERENCE TO SEQUENCE LISTING SUBMITTED ELECTRONICALLY

The official copy of the sequence listing is submitted electronically via EFS-Web as an ASCII formatted sequence listing with a file named 8472_SeqList.txt created on Oct. 8, 2020 and having a size of 344 kilobytes and is filed concurrently with the specification. The sequence listing contained in this ASCII formatted document is part of the specification and is herein incorporated by reference in its entirety.

### BACKGROUND

Certain species of microorganisms of the genus Bacillus are known to possess pesticidal activity against a range of insect pests including Lepidoptera, Diptera, Coleoptera, Hemiptera and others. Bacillus thuringiensis (Bt) and Bacillus popilliae are among the most successful biocontrol agents discovered to date. Insect pathogenicity has also been attributed to strains of B. larvae, B. lentimorbus, B. sphaericus and B. cereus. Microbial insecticides, particularly those obtained from Bacillus strains, have played an important role in agriculture as alternatives to chemical pest control.

Crop plants have been developed with enhanced insect resistance by genetically engineering crop plants to produce pesticidal proteins from Bacillus. For example, corn and cotton plants have been genetically engineered to produce pesticidal proteins isolated from strains of Bt. These genetically modified crops are now widely used in agriculture and have provided the farmer with an environmentally friendly alternative to traditional insect-control methods. While they have proven to be very successful commercially, these genetically modified, insect-resistant crop plants provide resistance to only a narrow range of the economically important insect pests. In some cases, insects can develop resistance to different insecticidal compounds, which raises the need to identify alternative biological control agents for pest control.

There has been a long felt need for environmentally friendly compositions and methods for controlling or eradicating insect pests of agricultural significance, i.e., methods that are selective, environmentally inert, non-persistent, and biodegradable, and that fit well into insect pest management schemes.

### SUMMARY

Some embodiments relate to a composition comprising a Pantoea agglomerans, wherein the Pantoea agglomerans has insecticidal activity. In some embodiments, the methods and compositions relate to a insecticidal bacterial strain comprising IPD126 gene or gene cluster. In some embodiments, the IPD126 gene comprises an amino acid sequence having at least 90% sequence identity to any one of SEQ ID NOs: 19-36. In some embodiments, the methods and compositions relate to bacterial strains comprising a 16S RNA sequence having at least 95% identity to any one of SEQ ID NOs: 37-39.

In one embodiment, the disclosure relates to a composition comprising a Pantoea agglomerans strain PMC3671E3-1 (NRRL Deposit No. B-67697), wherein the Pantoea agglomerans strain PMC3671E3-1 has insecticidal activity.

In one embodiment, the disclosure relates to a composition comprising a Pantoea agglomerans strain PMC3671E9-1 (NRRL Deposit No. B-67698), wherein the Pantoea agglomerans strain PMC3671E9-1 has insecticidal activity.

In one embodiment, the disclosure relates to a composition comprising a Pantoea agglomerans strain PMCJ4082D4-1 (NRRL Deposit No. B-67699), wherein the Pantoea agglomerans strain PMCJ4082D4-1 has insecticidal activity.

In yet another embodiment, the disclosure relates methods and compositions comprising a bacterial strain disclosed herein, or a progeny, mutant, or variant thereof, a fermentate produced from a strain disclosed herein progeny, mutant, or variant thereofin an effective amount to achieve an effect of inhibit growth of a plant pathogen, pest or insect. In another embodiment, the composition further comprises a biocontrol agent selected from the group consisting of bacteria, fungi, yeast, protozoans, viruses, entomopathogenic nematodes, botanical extracts, proteins, secondary metabolites, and inoculants.

In another embodiment, the compositions and methods disclosed herein further comprise one or more agrochemically active compounds selected from the group consisting of an insecticide, a fungicide, a bactericide, and a nematicide. In still another embodiment, the composition further comprises a compound selected from the group consisting of a safener, a lipo-chitooligosaccharide, an isoflavone, and a ryanodine receptor modulator.

In another embodiment, the compositions and methods comprise at least one at least one seed, plant, or plant part. In one embodiment, the seed, plant, or plant part is genetically modified.

In one embodiment, the compositions and methods inhibit the growth of one or more plant pathogens, pests, or insects including but not limited to bacteria, a fungus, a virus, protozoa, nematode, or an arthropod. In one embodiment, the compositions and methods inhibit the growth of an insect, including but not limited to a Coleopteran, Hemipteran, or Lepidopteran insect. In still another embodiment, the composition inhibits the growth of Diabrotica virgifera virgifera, Ostrinia nubilalis, Spodoptera frugiperda, Pseudoplusia includens, Anticarsia gemmatalis, Plutella xylostella, and/or Aphis fabae.

In another embodiment, the compositions and methods comprise an effective amount to provide pesticidal activity to bacteria, plants, plant cells, tissues and seeds. In another embodiment, the composition is an effective amount to provide pesticidal activity to Coleopteran or Lepidopteran insects. In still another embodiment, the composition is an effective amount to provide pesticidal activity to Diabrotica virgifera virgifera, Ostrinia nubilalis, Spodoptera frugiperda, Pseudoplusia includens, Anticarsia gemmatalis, Plutella xylostella, and/or Aphis fabae.

In another embodiment, the compositions and methods comprise in an effective amount to improve plant performance including but not limited to increased root formation, increased root mass, increased root function, increased shoot height, increased shoot function, increased flower bud presence, increased flower bud formation, increased seed germination, increased yield, increased total plant wet weight, and increased total plant dry weight.

In another embodiment, the disclosure relates to a method comprising applying a composition comprising a bacterial strain disclosed herein, or a progeny, mutant, or variant thereof, a fermentate produced from a strain disclosed herein, or a progeny, mutant, or variant thereof, a plant, plant part or soil in an effective amount to achieve an effect selected from the group consisting of: inhibit a plant pathogen, pest, or insect or to prevent damage to a plant by a pathogen, pest, or insect; improve plant performance; improve plant yield; improve plant vigor; increase phosphate availability; increase production of a plant hormone; increase root formation; increase shoot height in a plant, increase leaf length of a plant; increase flower bud formation of a plant; increase total plant fresh weight; increase total plant dry weight; and increase seed germination.

In another embodiment, the method further comprises applying a biocontrol agent, wherein the biocontrol agent is selected from the group consisting of bacteria, fungi, yeast, protozoans, viruses, entomopathogenic nematodes, botanical extracts, proteins, secondary metabolites, and inoculants.

In yet another embodiment, the method further comprises applying an agrochemically active compound selected from the group consisting of an insecticide, a fungicide, a bactericide, and a nematicide.

In still another embodiment, the method further comprises applying a compound selected from the group consisting of a safener, a lipo-chitooligosaccharide, an isoflavone, and a ryanodine receptor modulator.

In another embodiment, the method comprises applying the composition in an effective amount to inhibit growth of a plant pathogen, including but not limited to bacteria, a fungus, a nematode, an insect, a virus, and protozoa.

In one aspect compositions and methods for conferring pesticidal activity to bacteria, plants, plant cells, tissues and seeds are provided. Compositions include nucleic acid molecules encoding sequences for pesticidal and insecticidal polypeptides, vectors comprising those nucleic acid molecules, and host cells comprising the vectors. Compositions also include the pesticidal polypeptide sequences and antibodies to those polypeptides. Compositions also comprise transformed bacteria, plants, plant cells, tissues and seeds.

In another aspect methods are provided for producing the polypeptides and for using those polypeptides for controlling or killing a Hemipteran, Coleopteran, Lepidopteran, or nematode pests. The transgenic plants of the embodiments express one or more of the pesticidal sequences disclosed herein. In various embodiments, the transgenic plant further comprises one or more additional genes for insect resistance, for example, one or more additional genes for controlling Hemipteran, Coleopteran, Lepidopteran, or nematode pests. It will be understood by one of skill in the art that the transgenic plant may comprise any gene imparting an agronomic trait of interest.

In another aspect methods for detecting the nucleic acids and polypeptides of the embodiments in a sample are also included. A kit for detecting the presence of an IPD126 polypeptide or detecting the presence of a polynucleotide encoding an IPD126 polypeptide in a sample is provided. The kit may be provided along with all reagents and control samples necessary for carrying out a method for detecting the intended agent, as well as instructions for use.

In another aspect, the compositions and methods of the embodiments are useful for the production of organisms with enhanced pest resistance or tolerance. These organisms and compositions comprising the organisms are desirable for agricultural purposes. The compositions of the embodiments are also useful for generating altered or improved proteins that have pesticidal activity or for detecting the presence of IPD126 polypeptides.

### DESCRIPTION OF FIGURES

FIG. 1 shows IPD126 gene clusters in insecticidal strains of Pantoea agglomerans.

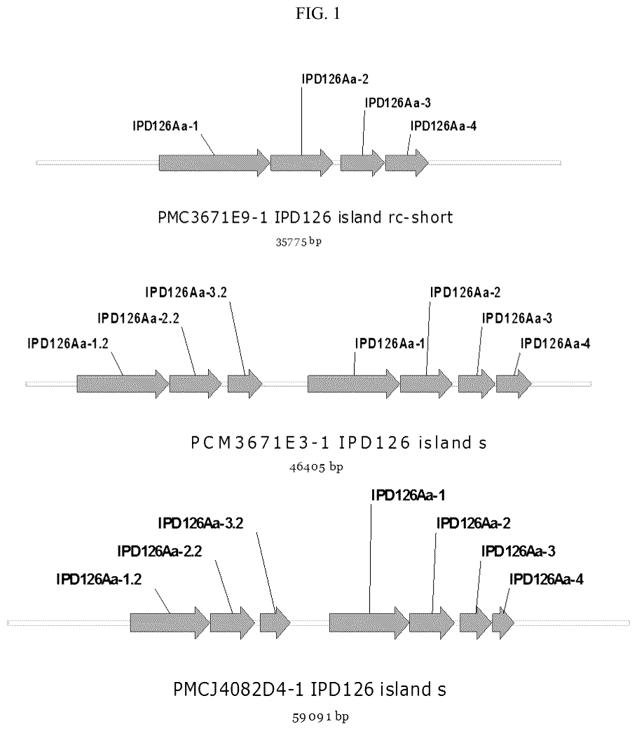

### DESCRIPTION OF THE SEQUENCES

The disclosure can be more fully understood from the following detailed description and the accompanying drawings and Sequence Listing that form a part of this application.

The sequence descriptions summarize the Sequence Listing attached hereto, which is hereby incorporated by reference. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

TABLE 1
Sequence Listing Description

| Strain_Gene | SEQ ID NO: |
|---|---|
| PMC3671E9-1_IPD126Aa-1 DNA | 1 |
| PMC3671E9-1_IPD126Aa-2 DNA | 2 |
| PMC3671E9-1_IPD126Aa-3 DNA | 3 |
| PMC3671E9-1_IPD126Aa-4 DNA | 4 |
| PCM3671E3-1_IPD126Aa-1 DNA | 5 |
| PCM3671E3-1_IPD126Aa-2 DNA | 6 |
| PCM3671E3-1_IPD126Aa-3 DNA | 7 |
| PCM3671E3-1_IPD126Aa-4 DNA | 8 |
| PCM3671E3-1_IPD126Aa-1.2 DNA | 9 |
| PCM3671E3-1_IPD126Aa-2.2 DNA | 10 |
| PCM3671E3-1_IPD126Aa-3.2 DNA | 11 |
| PMCJ4082D4-1_IPD126Aa-1 DNA | 12 |
| PMCJ4082D4-1_IPD126Aa-2 DNA | 13 |
| PMCJ4082D4-1_IPD126Aa-3 DNA | 14 |
| PMCJ4082D4-1_IPD126Aa-4 DNA | 15 |
| PMCJ4082D4-1_IPD126Aa-1.2 DNA | 16 |
| PMCJ4082D4-1_IPD126Aa-2.2 DNA | 17 |
| PMCJ4082D4-1_IPD126Aa-3.2 DNA | 18 |
| PMC3671E9-1_IPD126Aa-1 protein | 19 |
| PMC3671E9-1_IPD126Aa-2 Protein | 20 |
| PMC3671E9-1_IPD126Aa-3 Protein | 21 |
| PMC3671E9-1_IPD126Aa-4 Protein | 22 |
| PCM3671E3-1_IPD126Aa-1 protein | 23 |
| PCM3671E3-1_IPD126Aa-2 protein | 24 |
| PCM3671E3-1_IPD126Aa-3 Protein | 25 |
| PCM3671E3-1_IPD126Aa-4 Protein | 26 |
| PCM3671E3-1_IPD126Aa-1.2 protein | 27 |
| PCM3671E3-1_IPD126Aa-2.2 Protein | 28 |
| PCM3671E3-1_IPD126Aa-3.2 Protein | 29 |
| PMCJ4082D4-1_IPD126Aa-1 Protein | 30 |
| PMCJ4082D4-1_IPD126Aa-2 protein | 31 |
| PMCJ4082D4-1_IPD126Aa-3 protein | 32 |
| PMCJ4082D4-1_IPD126Aa-4 protein | 33 |
| PMCJ4082D4-1_IPD126Aa-1.2 protein | 34 |
| PMCJ4082D4-1_IPD126Aa-2.2 protein | 35 |
| PMCJ4082D4-1_IPD126Aa-3.2 protein | 36 |
| PMC3671E3-1-16S | 37 |
| PMC3671E9-1-16S | 38 |
| PMCJ4082D4-1-16S | 39 |

### DETAILED DESCRIPTION

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof, and so forth. All technical and scientific terms used herein have the same meaning as commonly understood to one of ordinary skill in the art to which this disclosure belongs unless clearly indicated otherwise.

As used herein, “administer” refers to the action of introducing a strain and/or a composition to an environment for pathogen, pest, or insect inhibition or to improve plant performance.

As used herein, the term “agrochemically active compounds” refers to any substance that is or may be customarily used for treating plants including, but not limited to, fungicides, bactericides, insecticides, acaricides, nematicides, molluscicides, safeners, plant growth regulators, and plant nutrients, as well as, microorganisms.

As used herein, a composition may be a liquid, a heterogeneous mixture, a homogeneous mixture, a powder, a solution, a dispersion or any combination thereof.

As used herein, “effective amount” refers to a quantity of a bacterial strain disclosed herein, or a progeny, mutant, or variant thereof, a fermentate produced from a strain disclosed herein progeny, mutant, or variant thereof sufficient to inhibit growth of a pathogenic microorganism or to impede the rate of growth of the pathogenic microorganism. In another embodiment, the term “effective amount” refers to a quantity of a bacterial strain disclosed herein, or a progeny, mutant, or variant thereof, a fermentate produced from a strain disclosed herein progeny, mutant, or variant thereof sufficient to improve plant performance. In another embodiment, the term “effective amount” refers to a quantity of a bacterial strain disclosed herein, or a progeny, mutant, or variant thereof, a fermentate produced from a strain disclosed herein progeny, mutant, or variant thereof, sufficient to control, kill, inhibit, and reduce the number, emergence, or growth of a pathogen, pest, or insect. In another embodiment, the term “effective amount” refers to a quantity of a bacterial strain disclosed herein, or a progeny, mutant, or variant thereof, a fermentate produced from a strain disclosed herein progeny, mutant, or variant thereof sufficient to prevent damage from a pathogen, pest, or insect. One skilled in the art will recognized that an effective amount of a bacterial strain disclosed herein, or a progeny, mutant, or variant thereof, a fermentate produced from a strain disclosed herein progeny, mutant, or variant thereof may not reduce the numbers of pathogens, pests or insects, but is effective in decreasing damage to plants and/or plant parts as a result of a pathogen, pest or insect. For example, a pesticidally effective amount may reduce pathogen, pest or insect emergence, or damage to seeds, roots, shoots, or foliage of plants that are treated compared to those that are untreated.

As used herein, “fermentate broth,” “fermentate,” or “fermented broth” refers to a media used to grow or ferment a bacterial strain disclosed herein. The bacterial strain may be removed from a media by filtration, sterilization, or other means. The leftover broth contains metabolites produced by a bacterial strain disclosed herein, which is collectively referred to as a “fermentate broth,” “fermentate,” or “fermented broth.”

As used herein, the term “inhibit” refers to destroy, prevent, reduce, resist, control, decrease, slow or otherwise interfere with the growth or survival of a pathogen, pest, or insect when compared to the growth or survival of the pathogen, pest, or insect in an untreated control. Any of the terms of inhibit, destroy, prevent, control, decrease, slow, interfere, resist, or reduce may be used interchangeably. In one embodiment, to “inhibit” is to destroy, prevent, control, reduce, resist, decrease, slow or otherwise interfere with the growth, emergence, or survival of a pathogen, pest, or insect by at least about 3% to at least about 100%, or any value in between for example at least about 10%, 15%, 20%, 25%, 30%, 35%, 40%, 45%, 50%, 55%, 60%, 65%, 70%, 75%, 80%, 85%, 90%, 95%, 99%, or 100% when compared to the growth or survival of the pathogen, pest, or insect in an untreated control. The amount of inhibition can be measured as described herein or by other methods known in the art. As used herein, “protects a plant from a pathogen, pest, or insect pest” is intended to mean the limiting or eliminating of the pathogen, pest, or insect related damage to a plant and/or plant part by, for example, inhibiting the ability of the pathogen, pest, or insect to grow, emerge, feed, and/or reproduce or by killing the pathogen, pest, or insect. As used herein, pesticidal and/or insecticidal activity refers to an activity of compound, composition, and or method that protects a plant and/or plant part from a pathogen, pest, or insect.

In some embodiments, inhibition a pathogen, pest, or insect lasts for or provides protection for greater than a day, two days, three days, four days, five days, six days, a week, two weeks, three weeks, a month or more after of a bacterial strain disclosed herein, or a progeny, mutant, or variant thereof, a fermentate produced from a strain disclosed herein progeny, mutant, or variant thereof disclosed herein is applied to subject material. In another embodiment, inhibition a pathogen, pest or insect lasts from one to seven days, from seven to 14 days, from 14 to 21 days, or from 21 to 30 days or more. In another embodiment, the inhibition of the growth of a pathogen, pest, or insect lasts for or provides protection for greater than the time from application to adult emergence of the pathogen, pest, or insect.

As used herein, the term “genetically modified” is intended to mean any species containing a genetic trait, loci, or sequence that was not found in the species or strain prior to manipulation. A genetically modified plant may be transgenic, cis-genic, genome edited, or bred to contain a new genetic trait, loci, or sequence. A genetically modified plant or bacteria may be prepared by means known to those skilled in the art, such as transformation by bombardment, by a gene editing technique such as Cas/CRISPR or TALENS, or by breeding techniques. As used herein, a “trait” is a new or modified locus or sequence of a genetically modified plant or bacteria, including but not limited to a transgenic plant or bacteria. In some embodiments, a bacterial strain disclosed herein, or a progeny, mutant, or variant thereof, may be edited. In some embodiments any bacterial strain maybe modified or edited to comprise an IPD126 gene. In some embodiments, the methods and compositions relate to an insecticidal bacterial strain comprising an IPD126 gene

As used herein, the term “environment of a plant or plant part” is intended to mean the area surrounding the plant or plant part, including but not limited to the soil, the air, or in-furrow. The environment of a plant or plant part may be in proximity, touching, adjacent to, or in the same field as the plant or plant part. The compositions described herein may be applied to the environment of the plant or plant part as a seed treatment, as a foliar application, as a granular application, as a soil application, or as an encapsulated application. As used herein, “in-furrow” is intended to mean within or near the area where a seed is planted. The compositions disclosed herein may be applied in-furrow concurrently or simultaneously with a seed. In another embodiment, the compositions disclosed herein may be applied sequentially, either before or after a seed is planted.

As used herein, the term “different mode of action” is used to refer to a pesticidal composition inhibiting a pathogen, pest, or insect through a pathway or receptor that is different from another pesticidal composition. As used herein, the term “different mode of action” includes the pesticidal effects of one or more pesticidal compositions to different binding sites (i.e., different toxin receptors and/or different sites on the same toxin receptor) in the gut membranes of insects or through the RNA interference pathway to different target genes.

As used herein, the term “pathogen, pest, or insect”includes but is not limited to pathogenic fungi, bacteria, mites, ticks, pathogenic microorganisms, and nematodes, as well as insect from the orders Coleoptera, Lepidoptera, Mallophaga, Homoptera, Hemiptera, Orthoptera. Thysanoptera, Dermaptera, Isoptera, Anoplura, Siphonaptera, Trichoptera, and others, including but not limited to Diabrotica virgifera virgifera, Diabrotica undecimpunctata howardi, and Diabrotica barberi.

Larvae of the order Lepidoptera include, but are not limited to, armyworms, cutworms, loopers and heliothines in the family Noctuidae Spodoptera frugiperda J E Smith (fall armyworm); S. exigua Hübner (beet armyworm); S. litura Fabricius (tobacco cutworm, cluster caterpillar); Mamestra configurata Walker (bertha armyworm); M. brassicae Linnaeus (cabbage moth); Agrotis ipsilon Hufnagel (black cutworm); A. orthogonia Morrison (western cutworm); A. subterranea Fabricius (granulate cutworm); Alabama argillacea Hübner (cotton leaf worm); Trichoplusia ni Hübner (cabbage looper); Pseudoplusia includens Walker (soybean looper); Anticarsia gemmatalis Hübner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messoria Harris (darksided cutworm); Earias insulana Boisduval (spiny bollworm); E vittella Fabricius (spotted bollworm); Helicoverpa armigera Hübner (American bollworm); H. zea Boddie (corn earworm or cotton bollworm); Melanchra picta Harris (zebra caterpillar); Egira (Xylomyges) curialis Grote (citrus cutworm); borers, casebearers, webworms, coneworms, and skeletonizers from the family Pyralidae Ostrinia nubilalis Hübner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus, (sorghum borer); Corcyra cephalonica Stainton (rice moth); Crambus caliginosellus Clemens (corn root webworm); C. teterrellus Zincken (bluegrass webworm); Cnaphalocrocis medinalis Guenée (rice leaf roller); Desmia funeralis Hübner (grape leaffolder); Diaphania hyalinata Linnaeus (melon worm); D. nitidalis Stoll (pickleworm); Diatraea grandiosella Dyar (southwestern corn borer), D. saccharalis Fabricius (surgarcane borer); Eoreuma loftini Dyar (Mexican rice borer); Ephestia elutella Hübner (tobacco (cacao) moth); Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia grisella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hübner (Indian meal moth); Scirpophaga incertulas Walker (yellow stem borer); Udea rubigalis Guenée (celery leaftier); and leafrollers, budworms, seed worms and fruit worms in the family Tortricidae Acleris gloverana Walsingham (Western blackheaded budworm); A. variana Fernald (Eastern blackheaded budworm); Archips argyrospila Walker (fruit tree leaf roller); A. rosana Linnaeus (European leaf roller); and other Archips species, Adoxophyes orana Fischer von Rösslerstamm (summer fruit tortrix moth); Cochylis hospes Walsingham (banded sunflower moth); Cydia latiferreana Walsingham (filbertworm); C. pomonella Linnaeus (coding moth); Platynota flavedana Clemens (variegated leafroller); P. stultana Walsingham (omnivorous leafroller); Lobesia botrana Denis & Schiffermiller (European grape vine moth); Spilonota ocellana Denis & Schiffermüller (eyespotted bud moth); Endopiza viteana Clemens (grape berry moth); Eupoecilia ambiguella Hübner (vine moth); Bonagota salubricola Meyrick (Brazilian apple leafroller); Grapholita molesta Busck (oriental fruit moth); Suleima helianthana Riley (sunflower bud moth); Argyrotaenia spp.; Choristoneura spp..

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Méneville (Chinese Oak Tussah Moth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Euproctis chrysorrhoea Linnaeus (browntail moth); Harrisina americana Guerin-Meneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M. sexta Haworth (tomato hornworm, tobacco hornworm); Operophtera brumata Linnaeus (winter moth); Paleacrita vernata Peck (spring cankerworm); Papilio cresphontes Cramer (giant swallowtail orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval and Leconte (Southern cabbageworm); Sabulodes aegrotata Guenée (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tula absoluta Meyrick (tomato leafminer); Yponomeuta padella Linnaeus (ermine moth); Heliothis subflexa Guenée; Malacosoma spp. and Orgyia spp.

Of interest are larvae and adults of the order Coleoptera including weevils from the families Anthribidae, Bruchidae and Curculionidae (including, but not limited to: Anthonomus grandis Boheman (boll weevil); Lissorhoptrus oryzophilus Kuschel (rice water weevil); Sitophilus granarius Linnaeus (granary weevil); S. oryzae Linnaeus (rice weevil); Hypera punctata Fabricius (clover leaf weevil); Cylindrocopturus adspersus LeConte (sunflower stem weevil); Smicronyx fulvus LeConte (red sunflower seed weevil); S. sordidus LeConte (gray sunflower seed weevil); Sphenophorus maidis Chittenden (maize billbug)); flea beetles, cucumber beetles, rootworms, leaf beetles, potato beetles and leafminers in the family Chrysomelidae (including, but not limited to: Leptinotarsa decemlineata Say (Colorado potato beetle); Diabrotica virgifera virgifera LeConte (western corn rootworm); D. barberi Smith and Lawrence (northern corn rootworm); D. undecimpunctata howardi Barber (southern corn rootworm); Chaetocnema pulicaria Melsheimer (corn flea beetle); Phyllotreta cruciferae Goeze (Crucifer flea beetle); Phyllotreta striolata (stripped flea beetle); Colaspis brunnea Fabricius (grape colaspis); Oulema melanopus Linnaeus (cereal leaf beetle); Zygogramma exclamationis Fabricius (sunflower beetle)); beetles from the family Coccinellidae (including, but not limited to: Epilachna varivestis Mulsant (Mexican bean beetle)); chafers and other beetles from the family Scarabaeidae (including, but not limited to: Popillia japonica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); Ligyrus gibbosus De Geer (carrot beetle)); carpet beetles from the family Dermestidae; wireworms from the family Elateridae, Eleodes spp., Melanotus spp.; Conoderus spp.; Limonius spp.; Agriotes spp.; Ctenicera spp.; Aeolus spp.; bark beetles from the family Scolytidae and beetles from the family Tenebrionidae.

Adults and immatures of the order Diptera are of interest, including leafminers Agromyza parvicornis Loew (corn blotch leafminer); midges (including, but not limited to: Contarinia sorghicola Coquillett (sorghum midge); Mayetiola destructor Say (Hessian fly); Sitodiplosis mosellana Géhin (wheat midge); Neolasioptera murtfeldtiana Felt, (sunflower seed midge)); fruit flies (Tephritidae), Oscinella frit Linnaeus (fruit flies); maggots (including, but not limited to: Delia platura Meigen (seedcorn maggot); D. coarctata Fallen (wheat bulb fly) and other Delia spp., Meromyza americana Fitch (wheat stem maggot); Musca domestica Linnaeus (house flies); Fannia canicularis Linnaeus, F. femoralis Stein (lesser house flies); Stomoxys calcitrans Linnaeus (stable flies)); face flies, horn flies, blow flies, Chrysomya spp.; Phormia spp. and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds) and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are adults and nymphs of the orders Hemiptera and Homoptera such as, but not limited to, adelgids from the family Adelgidae, plant bugs from the family Miridae, cicadas from the family Cicadidae, leafhoppers, Empoasca spp.; from the family Cicadellidae, planthoppers from the families Cixiidae, Flatidae, Fulgoroidea, Issidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Asterolecanidae, Coccidae, Dactylopiidae, Diaspididae, Eriococcidae Ortheziidae, Phoenicococcidae and Margarodidae, lace bugs from the family Tingidae, stink bugs from the family Pentatomidae, cinch bugs, Blissus spp.; and other seed bugs from the family Lygaeidae, spittlebugs from the family Cercopidae squash bugs from the family Coreidae and red bugs and cotton stainers from the family Pyrrhocoridae.

Agronomically important members from the order Homoptera further include, but are not limited to: Acyrthisiphon pisum Harris (pea aphid); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacorthum solani Kaltenbach (foxglove aphid); Chaetosiphonfragaefolii Cockerell (strawberry aphid); Diuraphis noxia Kurdjumov/Mordvilko (Russian wheat aphid); Dysaphis plantaginea Paaserini (rosy apple aphid); Eriosoma lanigerum Hausmann (woolly apple aphid); Brevicoryne brassicae Linnaeus (cabbage aphid); Hyalopterus pruni Geoffroy (mealy plum aphid); Lipaphis erysimi Kaltenbach (turnip aphid); Metopolophium dirrhodum Walker (cereal aphid); Macrosiphum euphorbiae Thomas (potato aphid); Myzus persicae Sulzer (peach-potato aphid, green peach aphid); Nasonovia ribisnigri Mosley (lettuce aphid); Pemphigus spp. (root aphids and gall aphids); Rhopalosiphum maidis Fitch (corn leaf aphid); R padi Linnaeus (bird cherry-oat aphid); Schizaphis graminum Rondani (greenbug); Siphaflava Forbes (yellow sugarcane aphid); Sitobion avenae Fabricius (English grain aphid); Therioaphis maculata Buckton (spotted alfalfa aphid); Toxoptera aurantih Boyer de Fonscolombe (black citrus aphid) and T. citricida Kirkaldy (brown citrus aphid); Melanaphis sacchari (sugarcane aphid); Adelges spp. (adelgids); Phylloxera devastatrix Pergande (pecan phylloxera); Bemisia tabaci Gennadius (tobacco whitefly, sweetpotato whitefly); B. argentifolii Bellows & Perring (silverleaf whitefly); Dialeurodes citri Ashmead (citrus whitefly); Trialeurodes abutiloneus (bandedwinged whitefly) and T. vaporariorum Westwood (greenhouse whitefly); Empoasca fabae Harris (potato leafhopper); Laodelphax striatellus Fallen (smaller brown planthopper); Macrolestes quadrilineatus Forbes (aster leafhopper); Nephotettix cinticeps Uhler (green leafhopper); N. nigropictus Stil (rice leafhopper); Nilaparvata lugens Stil (brown planthopper); Peregrinus maidis Ashmead (corn planthopper); Sogatellafurcifera Horvath (white-backed planthopper); Sogatodes orizicola Muir (rice delphacid); Typhlocyba pomaria McAtee (white apple leafhopper); Erythroneoura spp. (grape leafhoppers); Magicicada septendecim Linnaeus (periodical cicada); Icerya purchasi Maskell (cottony cushion scale); Quadraspidiotus perniciosus Comstock (San Jose scale); Planococcus citri Risso (citrus mealybug); Pseudococcus spp. (other mealybug complex); Cacopsylla pyricola Foerster (pear psylla); Trioza diospyri Ashmead (persimmon psylla).

Agronomically important species of interest from the order Hemiptera include, but are not limited to: Acrosternum hilare Say (green stink bug); Anasa tristis De Geer (squash bug); Blissus leucopterus leucopterus Say (chinch bug); Corythuca gossypii Fabricius (cotton lace bug); Cyrtopeltis modesta Distant (tomato bug); Dysdercus suturellus Herrich-Schiffer (cotton stainer); Euschistus servus Say (brown stink bug); E variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnar Fabricius (rice stink bug); Oncopeltus fasciatus Dallas (large milkweed bug); Pseudatomoscelis seriatus Reuter (cotton fleahopper).

Furthermore, embodiments may be effective against Hemiptera such, Calocoris norvegicus Gmelin (strawberry bug); Orthops campestris Linnaeus; Plesiocoris rugicollis Fallen (apple capsid); Cyrtopeltis modestus Distant (tomato bug); Cyrtopeltis notatus Distant (suckfly); Spanagonicus albofasciatus Reuter (whitemarked fleahopper); Diaphnocoris chlorionis Say (honeylocust plant bug); Labopidicola allii Knight (onion plant bug); Pseudatomoscelis seriatus Reuter (cotton fleahopper); Adelphocoris rapidus Say (rapid plant bug); Poecilocapsus lineatus Fabricius (four-lined plant bug); Nysius ericae Schilling (false chinch bug); Nysius raphanus Howard (false chinch bug); Nezara viridula Linnaeus (Southern green stink bug); Eurygaster spp.; Coreidae spp.; Pyrrhocoridae spp.; Tinidae spp.; Blostomatidae spp.; Reduviidae spp. and Cimicidae spp.

Also included are adults and larvae of the order Acari (mites) such as Aceria tosichella Keifer (wheat curl mite); Petrobia latens Muller (brown wheat mite); spider mites and red mites in the family Tetranychidae, Panonychus ulmi Koch (European red mite); Tetranychus urticae Koch (two spotted spider mite); (T. mcdanieli McGregor (McDaniel mite); T. cinnabarinus Boisduval (carmine spider mite); T. turkestani Ugarov & Nikolski (strawberry spider mite); flat mites in the family Tenuipalpidae, Brevipalpus lewisi McGregor (citrus flat mite); rust and bud mites in the family Eriophyidae and other foliar feeding mites and mites important in human and animal health, i.e., dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick) and scab and itch mites in the families Psoroptidae, Pyemotidae and Sarcoptidae.

Insect pests of the order Thysanura are of interest, such as Lepisma saccharina Linnaeus (silverfish); Thermobia domestica Packard (firebrat).

Additional arthropod pests covered include: spiders in the order Araneae such as Loxosceles reclusa Gertsch and Mulaik (brown recluse spider) and the Latrodectus mactans Fabricius (black widow spider) and centipedes in the order Scutigeromorpha such as Scutigera coleoptrata Linnaeus (house centipede).

Insect pest of interest include the superfamily of stink bugs and other related insects including but not limited to species belonging to the family Pentatomidae (Nezara viridula, Halyomorpha halys, Piezodorus guildini, Euschistus servus, Acrosternum hilare, Euschistus heros, Euschistus tristigmus, Acrosternum hilare, Dichelopsfurcatus, Dichelops melacanthus, and Bagrada hilaris (Bagrada Bug)), the family Plataspidae (Megacopta cribraria —Bean plataspid) and the family Cydnidae (Scaptocoris castanea—Root stink bug) and Lepidoptera species including but not limited to: diamond-back moth, e.g., Helicoverpa zea Boddie; soybean looper, e.g., Pseudoplusia includens Walker and velvet bean caterpillar e.g., Anticarsia gemmatalis HObner.

Methods for measuring pesticidal activity are well known in the art. See, for example, Czapla and Lang, (1990) J. Econ. Entomol. 83:2480-2485; Andrews, et al., (1988) Biochem. J. 252:199-206; Marrone, et al., (1985) J. of Economic Entomology 78:290-293 and U.S. Pat. No. 5,743,477. Generally, the pesticide is mixed and used in feeding assays. See, for example Marrone, et al., (1985) J. of Economic Entomology 78:290-293. Such assays can include contacting plants with one or more pests and determining the plant's ability to survive and/or cause the death of the pests.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence x 100).

In some embodiments, an IPD126 polypeptide comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of any one of SEQ ID NOs: 19-36. In some embodiments, a nucleic acid sequence encoding an IPD126 polynucletoide sequence having at least about 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of any one of SEQ ID NOs: 1-18.

As used herein, the term “plant” refers to all plants, plant parts, seed, and plant populations, such as desirable and undesirable wild plants, cultivars, transgenic plants, and plant varieties (whether or not protectable by plant variety or plant breeder's rights). Cultivars and plant varieties can be plants obtained by conventional propagation and breeding methods that can be assisted or supplemented by one or more biotechnological methods such as by use of double haploids, protoplast fusion, random and directed mutagenesis, molecular or genetic markers or by bioengineering and genetic engineering methods.

The embodiments disclosed herein may generally be used for any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Fleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables ornamentals, and conifers.

As used herein, the term “plant parts” refers to all above ground and below ground parts and organs of plants such as shoot, leaf, blossom and root, whereby for example leaves, needles, stems, branches, blossoms, fruiting bodies, fruits and seeds, as well as roots, tubers, corms and rhizomes are included. Crops and vegetative and generative propagating material, for example, cuttings, corms, rhizomes, tubers, runners and seeds are also plant parts.

As used herein, the term “viable” refers to a microbial cell, propagule, or spore that is metabolically active or able to differentiate. Thus, propagules, such as spores, are “viable” when they are dormant and capable of germinating.

The embodiments disclosed herein relate to a Pantoea agglomerans strain PMC3671E3-1 (NRRL Deposit No. B-67697), a Pantoea agglomerans strain PMC3671E9-1 (NRRL Deposit No. B-67698), or a Pantoea agglomerans strain PMCJ4082D4-1 (NRRL Deposit No. B-67699); and/or a fermentate produced from a growth medium comprising a Pantoea agglomerans strain PMC3671E3-1 (NRRL Deposit No. B-67697), a Pantoea agglomerans strain PMC3671E9-1 (NRRL Deposit No. B-67698), or a Pantoea agglomerans strain PMCJ4082D4-1 (NRRL Deposit No. B-67699. In one embodiment the bacterial strain disclosed herein, or a progeny, mutant, or variant thereof, and/or a fermentate produced from a strain disclosed herein progeny, mutant, or variant thereof; compositions and methods find use in inhibiting, controlling, or killing a pathogen, pest, or insect, including, but is not limited to, fungi, pathogenic fungi, bacteria, mites, ticks, pathogenic microorganisms, and nematodes, as well as insects from the orders Coleoptera, Diptera, Hymenoptera, Lepidoptera, Mallophaga, Homoptera, Hemiptera Orthroptera, Thysanoptera, Dermaptera, Isoptera, Anoplura, Siphonaptera, Trichoptera, etc., particularly Coleoptera, including but not limited to Diabrotica virgifera virgifera, Diabrotica undecimpunctata howardi, and Diabrotica barberi, and for producing compositions with pesticidal activity.

The Pantoea agglomerans strain PMC3671E3-1 (NRRL Deposit No. B-67697), Pantoea agglomerans strain PMC3671E9-1 (NRRL Deposit No. B-67698), and Pantoea agglomerans strain PMCJ4082D4-1 (NRRL Deposit No. B-67699) were deposited on Nov. 9, 2018 at the Agricultural Research Service Culture Collection (NRRL), 1815 North University Street, Peoria, Ill., 61604. The deposits were made under the provisions of the Budapest Treaty on the International Recognition of the Deposit of Microorganisms for the Purposes of Patent Procedure.

Further, these deposits will be maintained under the terms of the Budapest Treaty on the International Recognition of the Deposit of Microorganisms for the Purposes of Patent Procedure. Access to these deposits will be available during the pendency of the application to the Commissioner of Patents and Trademarks and persons determined by the Commissioner to be entitled thereto upon request. Upon allowance of any claims in the application, the Applicant will make available to the public, pursuant to 37 C.F.R. § 1.808, sample(s) of the deposits. The deposits will be maintained in the NRRL depository, which is a public depository, for a period of 30 years, or 5 years after the most recent request, or for the enforceable life of the patent, whichever is longer, and will be replaced if it becomes nonviable during that period. Additionally, Applicant has satisfied all the requirements of 37 C.F.R. §§ 1.801-1.809, including providing an indication of the viability of the sample upon deposit.

Some embodiments relate to compositions comprising or consisting of or consisting essentially of a bacterial strain disclosed herein, or a progeny, mutant, or variant thereof, a fermentate produced from a strain disclosed herein progeny, mutant, or variant thereof. In one embodiment, the compositions are biologically pure cultures of the strain disclosed herein.

Some embodiments relate to a composition comprising a bacterial strain disclosed herein, or a progeny, mutant, or variant thereof, a fermentate produced from a strain disclosed herein progeny, mutant, or variant thereof disclosed herein and one or more compounds or agents selected from the group consisting of: agrochemically active compounds, biocontrol agents, lipo-chitooligosaccharide compounds (LCOs), isoflavones, quinazolines, insecticidal compounds, azolopyrimidinylamines, polymeric compounds, ionic compound, substituted thiophenes, substituted dithiines, fluopyramm, enaminocarbonyl compounds, strigolactone compound, and dithiino-tetracarboximide compounds.

A further embodiment relates to the use of a first composition comprising a bacterial strain disclosed herein, or a progeny, mutant, or variant thereof, a fermentate produced from a strain disclosed herein progeny, mutant, or variant thereof disclosed herein and a second composition comprising one or more compounds or agents selected from the group consisting of: agrochemically active compounds, biocontrol agents, lipo-chitooligosaccharide compounds (LCOs), isoflavones, quinazolines, insecticidal compound, azolopyrimidinylamine, polymeric compounds, ionic compound, substituted thiophenes, substituted dithiines, fluopyramm, enaminocarbonyl compounds, strigolactone compound, and dithiino-tetracarboximide compounds.

In one embodiment, the disclosure relates to a composition comprising a bacterial strain disclosed herein, or a progeny, mutant, or variant thereof, a fermentate produced from a strain disclosed herein progeny, mutant, or variant thereof disclosed herein and one or more biocontrol agents. As used herein, the term “biocontrol agent” (“BCA”) includes bacteria, fungi or yeasts, protozoans, viruses, entomopathogenic nematodes, and botanical extracts, or products produced by microorganisms including proteins or secondary metabolite, and inoculants that have one or both of the following characteristics: (1) inhibits or reduces plant infestation and/or growth of pathogens, pests, or insects, including but not limited to pathogenic fungi, bacteria, and nematodes, as well as arthropod pests such as insects, arachnids, chilopods, diplopods, or that inhibits plant infestation and/or growth of a combination of plant pathogens, pests, or insects; (2) improves plant performance; (3) improves plant yield; (4) improves plant vigor; and (5) improves plant health.

In one embodiment, the disclosure relates to a composition comprising a bacterial strain disclosed herein, or a progeny, mutant, or variant thereof, a fermentate produced from a strain disclosed herein progeny, mutant, or variant thereof disclosed herein and one or more agrochemically active compounds. Agrochemically active compounds are substances that are or may be used for treating a seed, a plant, plant part, or the environment of the seed or plant or plant part including but not limited to fungicides, bactericides, insecticides, acaricides, nematicides, molluscicides, safeners, plant growth regulators, plant nutrients, chemical entities with a known mechanism of action, additional microorganisms, and biocontrol agents.

In another embodiment, the disclosure relates to a first composition comprising a bacterial strain disclosed herein, or a progeny, mutant, or variant thereof, a fermentate produced from a strain disclosed herein progeny, mutant, or variant thereof and a second composition comprising one or more agrochemically active compounds, wherein the first and second composition may inhibit plant pathogens, pests, or insects and/or improve plant performance.

In one embodiment, the first and second compositions can be applied at the same time to a seed, a plant, plant part, or the environment of the plant. In another embodiment, the first composition can be applied to the seed followed by application of the second composition to the seed. In yet another embodiment, the second composition can be applied to the seed followed by application of the first composition to the seed.

In another embodiment, the first composition can be applied to the plant or plant part followed by application of the second composition to the plant or plant part. In yet another embodiment, the second composition can be applied to the plant or plant part followed by application of the first composition to the plant or plant part.

In another embodiment, the first composition can be applied to the seed and the second composition applied to the plant or plant part. In yet another embodiment, the second composition can be applied to the seed and the first composition applied to the plant or plant part.

In another embodiment, the first composition may be planted on or near the seed in a field. In yet another embodiment, the second composition can be applied to the seed and the first composition applied to the plant or plant part.

In one embodiment, the disclosure relates to the use of a bacterial strain disclosed herein, or a progeny, mutant, or variant thereof, a fermentate produced from a strain disclosed herein, progeny, mutant, or variant thereof, disclosed herein with a composition comprising an insecticidal protein from Pseudomonas sp. such as PSEEN3174 (Monalysin; (2011) PLoS Pathogens 7:1-13); from Pseudomonas protegens strain CHAO and Pf-5 (previously fluorescens) (Pechy-Tarr, (2008) Environmental Microbiology 10:2368-2386; GenBank Accession No. EU400157); from Pseudomonas Taiwanensis (Liu, et al., (2010)J Agric. Food Chem., 58:12343-12349) and from Pseudomonas pseudoalcligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxicology Journal, 3:101-118 and Morgan, et al., (2001) Applied and Envir. Micro. 67:2062-2069); U.S. Pat. Nos. 6,048,838, and 6,379,946; a PIP-1 polypeptide of U.S. Pat. No. 9,688,730; an AfIP-1A and/or AfIP-1B polypeptide of U.S. Pat. No. 9,475,847; a PIP-47 polypeptide of US Publication Number US20160186204; an IPD045 polypeptide, an IPD064 polypeptide, an IPD074 polypeptide, an IPD075 polypeptide, and an IPD077 polypeptide of PCT Publication Number WO 2016/114973; an IPD080 polypeptide of PCT Serial Number PCT/US17/56517; an IPD078 polypeptide, an IPD084 polypeptide, an IPD085 polypeptide, an IPD086 polypeptide, an IPD087 polypeptide, an IPD088 polypeptide, and an IPD089 polypeptide of Serial Number PCT/US17/54160; PIP-72 polypeptide of US Patent Publication Number US20160366891; a PtIP-50 polypeptide and a PtIP-65 polypeptide of US Publication Number US20170166921; an IPD098 polypeptide, an IPD059 polypeptide, an IPD108 polypeptide, an IPD109 polypeptide of U.S. Ser. No. 62/521,084; a PtIP-83 polypeptide of US Publication Number US20160347799; a PtIP-96 polypeptide of US Publication Number US20170233440; an IPD079 polypeptide of PCT Publication Number WO2017/23486; an IPD082 polypeptide of PCT Publication Number WO 2017/105987, an IPDO90 polypeptide of Serial Number PCT/US17/30602, an IPD093 polypeptide of U.S. Ser. No. 62/434,020; an IPD103 polypeptide of Serial Number PCT/US17/39376; an IPD101 polypeptide of U.S. Ser. No. 62/438,179; an IPD121 polypeptide of US Ser. No. 62/508,514; and 8-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry 51 and Cry55 classes of 6-endotoxin genes and the B. thuringiensis cytolytic Cyt1 and Cyt2 genes. Other Cry proteins are well known to one skilled in the art (see, Crickmore, et al., “Bacillus thuringiensis toxin nomenclature” (2011), at lifesci.sussex.ac.uk/home/Neil_Crickmore/Bt/which can be accessed on the world-wide web using the “www” prefix). The insecticidal activity of Cry proteins is well known to one skilled in the art (for review, see, van Frannkenhuyzen, (2009) J. Invert. Path. 101:1-16).

In one embodiment the composition comprises a silencing element of one or more polynucleotides of interest resulting in suppression of one or more target pathogen, pest, or insect polypeptides. By “silencing element” is it intended to mean a polynucleotide which when contacted by or ingested by a pest, is capable of reducing or eliminating the level or expression of a target polynucleotide or the polypeptide encoded thereby. The silencing element employed can reduce or eliminate the expression level of the target sequence by influencing the level of the target RNA transcript or, alternatively, by influencing translation and thereby affecting the level of the encoded polypeptide. Silencing elements may include, but are not limited to, a sense suppression element, an antisense suppression element, a double stranded RNA, a siRNA, an amiRNA, a miRNA, or a hairpin suppression element.

Nucleic acid molecules including silencing elements for targeting the vacuolar ATPase H subunit, useful for controlling a coleopteran pest population and infestation as described in US Patent Application Publication 2012/0198586. PCT Publication WO 2012/055982 describes ribonucleic acid (RNA or double stranded RNA) that inhibits or down regulates the expression of a target gene that encodes: an insect ribosomal protein such as the ribosomal protein L19, the ribosomal protein L40 or the ribosomal protein S27A; an insect proteasome subunit such as the Rpn6 protein, the Pros 25, the Rpn2 protein, the proteasome beta 1 subunit protein or the Pros beta 2 protein; an insect β-coatomer of the COPI vesicle, the γ-coatomer of the COPI vesicle, the β′-coatomer protein or the ζ-coatomer of the COPI vesicle; an insect Tetraspanine 2 A protein which is a putative transmembrane domain protein; an insect protein belonging to the actin family such as Actin 5C; an insect ubiquitin-5E protein; an insect Sec23 protein which is a GTPase activator involved in intracellular protein transport; an insect crinkled protein which is an unconventional myosin which is involved in motor activity; an insect crooked neck protein which is involved in the regulation of nuclear alternative mRNA splicing; an insect vacuolar H+-ATPase G-subunit protein and an insect Tbp-1 such as Tat-binding protein. PCT publication WO 2007/035650 describes ribonucleic acid (RNA or double stranded RNA) that inhibits or down regulates the expression of a target gene that encodes Snf7. US Patent Application publication 2011/0054007 describes polynucleotide silencing elements targeting RPS10. US Patent Application publication 2014/0275208 describes polynucleotide silencing elements targeting RyanR and PAT3. US Patent Application Publications 2012/029750, US 20120297501, and 2012/0322660 describe interfering ribonucleic acids (RNA or double stranded RNA) that functions upon uptake by an insect pest species to down-regulate expression of a target gene in said insect pest, wherein the RNA comprises at least one silencing element wherein the silencing element is a region of double-stranded RNA comprising annealed complementary strands, one strand of which comprises or consists of a sequence of nucleotides which is at least partially complementary to a target nucleotide sequence within the target gene. US Patent Application Publication 2012/0164205 describe potential targets for interfering double stranded ribonucleic acids for inhibiting invertebrate pests including: a Chd3 Homologous Sequence, a Beta-Tubulin Homologous Sequence, a 40 kDa V-ATPase Homologous Sequence, a EF1α Homologous Sequence, a 26S Proteosome Subunit p28 Homologous Sequence, a Juvenile Hormone Epoxide Hydrolase Homologous Sequence, a Swelling Dependent Chloride Channel Protein Homologous Sequence, a Glucose-6-Phosphate 1-Dehydrogenase Protein Homologous Sequence, an Act42A Protein Homologous Sequence, a ADP-Ribosylation Factor 1 Homologous Sequence, a Transcription Factor IIB Protein Homologous Sequence, a Chitinase Homologous Sequences, a Ubiquitin Conjugating Enzyme Homologous Sequence, a Glyceraldehyde-3-Phosphate Dehydrogenase Homologous Sequence, an Ubiquitin B Homologous Sequence, a Juvenile Hormone Esterase Homolog, and an Alpha Tubulin Homologous Sequence.

Some embodiments comprise an additional component, which may be a carrier, an adjuvant, a solubilizing agent, a suspending agent, a diluent, an oxygen scavenger, an antioxidant, a food material, an anti-contaminant agent, or combinations thereof.

In another embodiment, the additional component(s) may be required for the application to which the strain or composition is to be utilized. For example, if the strain or composition is to be utilized on, or in, an agricultural product, the additional component(s) may be an agriculturally acceptable carrier, excipient, or diluent. Likewise, if the strain or composition is to be utilized on, or in, a foodstuff the additional component(s) may be an edible carrier, excipient or diluent. In one aspect, the one or more additional component(s) is a carrier, excipient, or diluent. “Carriers” or “vehicles” mean materials suitable for compound administration and include any such material known in the art such as, for example, any liquid, gel, solvent, liquid diluent, solubilizer, or the like, which is non-toxic and does not interact with any components of the composition in a deleterious manner.

Examples of nutritionally acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Examples of excipients include but are not limited to: microcrystalline cellulose and other celluloses, lactose, sodium citrate, calcium carbonate, dibasic calcium phosphate, glycine, starch, milk sugar, and high molecular weight polyethylene glycols.

Examples of diluents include but are not limited to: water, ethanol, propylene glycol and glycerin, and combinations thereof.

The other components may be used simultaneously (e.g. when they are in admixture together or even when they are delivered by different routes) or sequentially (e.g. they may be delivered by different routes).

The composition or its diluent may also contain chelating agents such as EDTA, citric acid, tartaric acid, etc. Moreover, the composition or its diluent may contain active agents selected from fatty acids esters, such as mono- and diglycerides, non-ionic surfactants, such as polysorbates, phospholipids, etc. Emulsifiers may enhance the stability of the composition, especially after dilution.

The bacterial strain disclosed herein, or a progeny, mutant, or variant thereof, a fermentate produced from a strain disclosed herein progeny, mutant, or variant thereof may be used in any suitable form—whether when alone or when present in a composition. The compositions may be formulated in any suitable way to ensure that the composition comprises an active compound(s) of interest.

The bacterial strain disclosed herein, or a progeny, mutant, or variant thereof, a fermentate produced from a strain disclosed herein progeny, mutant, or variant thereof compositions thereof may be in the form of a dry powder that can be sprinkled on or mixed in with a product. The compositions in the form of a dry powder may include an additive such as microcrystalline cellulose, gum tragacanth, gelatin, starch, lactose, alginic acid, Primogel, or corn starch (which can be used as a disintegrating agent).

In yet another embodiment, a bacterial strain disclosed herein, or a progeny, mutant, or variant thereof, a fermentate produced from a strain disclosed herein progeny, mutant, or variant thereof compositions disclosed herein can be a spray-dried fermentate re-suspended in H₂O to a percentage selected from the following: 0.05-1, 1-3, 3-5, 5-7, 7-10, 10-15, 15-20, and greater than 20%. In another embodiment, one or more than one clarification step(s) can be performed prior to spray-drying.

In one embodiment, the compositions disclosed herein can comprise concentrated, dried propagules, from the strain disclosed herein. In one embodiment, compositions can be in the range of 1×10³ to 1×10¹³ CFU/g.

In one embodiment, the compositions disclosed herein can be applied in wet or partially or completely desiccated form or in slurry, gel, or other form.

In at least some embodiments, the compositions disclosed herein can be freeze-dried or lypholized. In at least some embodiments, the compositions can be mixed with a carrier. The carrier includes but is not limited to whey, maltodextrin, sucrose, dextrose, limestone (calcium carbonate), rice hulls, yeast culture, dried starch, clay, and sodium silico aluminate. The compositions can also be used with or without preservatives and in concentrated, un-concentrated, or diluted form. In one embodiment, the compositions can be in the form of a pellet or a biologically pure pellet.

The compositions described herein can be added to one or more carrier. Where used, the carrier(s) and the compositions can be added to a ribbon or paddle mixer and mixed for about 15 minutes, although the timing can be increased or decreased. The components are blended such that a uniform mixture of the culture and carrier(s) is produced. The final product is preferably a dry, flowable powder.

In one embodiment, the compositions may be formulated as a liquid, a dry powder, or a granule. The dry powder or granules may be prepared by means known to those skilled in the art, such as, in top-spray fluid bed coater, in a bottom spray Wurster, or by drum granulation (e.g. high sheer granulation), extrusion, pan coating or in a micro-ingredients mixer.

In another embodiment, the compositions disclosed herein may be provided as a spray-dried or freeze-dried powder.

In yet another embodiment, the compositions are in a liquid formulation. Such liquid consumption may contain one or more of the following: a buffer, salt, sorbitol, and/or glycerol.

In one embodiment, the compositions disclosed herein may be formulated with at least one physiologically acceptable carrier selected from at least one of maltodextrin, calcined (illite) clay, limestone (calcium carbonate), cyclodextrin, wheat or a wheat component, sucrose, starch, Na₂SO₄, Talc, PVA, sorbitol, benzoate, sorbiate, glycerol, sucrose, propylene glycol, 1,3-propane diol, glucose, parabens, sodium chloride, citrate, acetate, phosphate, calcium, metabisulfite, formate and mixtures thereof.

In one embodiment, the compositions disclosed herein may be formulated by encapsulation technology to improve stability and as a way to protect the compositions from seed applications. In one embodiment the encapsulation technology may comprise a bead polymer for timed release of the compositions over time. In one embodiment, the encapsulated compositions may be applied in a separate application of beads in-furrow to the seeds. In another embodiment, the encapsulated compositions may be co-applied along with seeds simultaneously.

The coating agent usable for the sustained release microparticles of an encapsulation embodiment may be a substance which is useful for coating the microgranular form with the substance to be supported thereon. Any coating agent which can form a coating difficultly permeable for the supported substance may be used in general, without any particular limitation. For example, higher saturated fatty acid, wax, thermoplastic resin, thermosetting resin and the like may be used.

Examples of useful higher saturated fatty acid include stearic acid, zinc stearate, stearic acid amide and ethylenebis-stearic acid amide; those of wax include synthetic waxes such as polyethylene wax, carbon wax, Hoechst wax, and fatty acid ester; natural waxes such as carnauba wax, bees wax and Japan wax; and petroleum waxes such as paraffin wax and petrolatum. Examples of thermoplastic resin include polyolefins such as polyethylene, polypropylene, polybutene and polystyrene; vinyl polymers such as polyvinyl acetate, polyvinyl chloride, polyvinylidene chloride, polyacrylic acid, polymethacrylic acid, polyacrylate and polymethacrylate; diene polymers such as butadiene polymer, isoprene polymer, chloroprene polymer, butadiene-styrene copolymer, ethylene-propylene-diene copolymer, styrene-isoprene copolymer, MMA-butadiene copolymer and acrylonitrile-butadiene copolymer; polyolefin copolymers such as ethylene-propylene copolymer, butene-ethylene copolymer, butene-propylene copolymer, ethylene-vinyl acetate copolymer, ethylene-acrylic acid copolymer, styreneacrylic acid copolymer, ethylene-methacrylic acid copolymer, ethylene-methacrylic ester copolymer, ethylene-carbon monoxide copolymer, ethylene-vinyl acetate-carbon monoxide copolymer, ethylene-vinyl acetate-vinyl chloride copolymer and ethylene-vinyl acetate-acrylic copolymer; and vinyl chloride copolymers such as vinyl chloride-vinyl acetate copolymer and vinylidene chloride-vinyl chloride copolymer. Examples of thermosetting resin include polyurethane resin, epoxy resin, alkyd resin, unsaturated polyester resin, phenolic resin, urea-melamine resin, urea resin and silicone resin. Of those, thermoplastic acrylic ester resin, butadienestyrene copolymer resin, thermosetting polyurethane resin and epoxy resin are preferred, and among the preferred resins, particularly thermosetting polyurethane resin is preferred. These coating agents can be used either singly or in combination of two or more kinds.

In one embodiment, the compositions may include a seed, a part of a seed, a plant, or a plant part.

All plants, plant parts, seeds or soil may be treated in accordance with the compositions and methods disclosed herein. The compositions disclosed herein may include a plant, a plant part, a seed, a seed part, or soil. The compositions and methods disclosed herein may be applied to the seed, the plant or plant parts, the fruit, or the soil in which the plants grow.

Some embodiments relate to a method for reducing plant pathogen, pest, or insect damage to a plant or plant part comprising: (a) treating a seed with a composition disclosed herein prior to planting. In another embodiment, the method further comprises: (b) treating a plant part obtained from the seed with a composition disclosed herein. The composition used in step (a) may be the same or different than the composition used in step (b).

Some embodiments relate to a method for reducing plant pathogen, pest, or insect damage to a plant or plant part comprising: (a) treating the soil surrounding a seed or plant a bacterial strain disclosed herein, or a progeny, mutant, or variant thereof, a fermentate produced from a strain disclosed herein progeny, mutant, or variant thereof. In another embodiment, the method further comprises: (b) treating a plant part with a bacterial strain disclosed herein, or a progeny, mutant, or variant thereof, a fermentate produced from a strain disclosed herein progeny, mutant, or variant thereof disclosed herein. The bacterial strain disclosed herein, or a progeny, mutant, or variant thereof, a fermentate produced from a strain disclosed herein progeny, mutant, or variant thereof used in step (a) may be the same or different than a bacterial strain disclosed herein, or a progeny, mutant, or variant thereof, a fermentate produced from a strain disclosed herein progeny, mutant, or variant thereof used in step (b).

Some embodiments relate to a method for reducing plant pathogen, pest, or insect damage to a plant or plant part comprising: (a) treating a seed prior to planting with a bacterial strain disclosed herein, or a progeny, mutant, or variant thereof, a fermentate produced from a strain disclosed herein progeny, mutant, or variant thereof disclosed herein. In another embodiment, the method further comprises: (b) treating the soil surrounding the seed or plant with a bacterial strain disclosed herein, or a progeny, mutant, or variant thereof, a fermentate produced from a strain disclosed herein progeny, mutant, or variant thereof disclosed herein. In still another embodiment, the method further comprises: (c) treating a plant part of a plant produced from the seed with a bacterial strain disclosed herein, or a progeny, mutant, or variant thereof, a fermentate produced from a strain disclosed herein progeny, mutant, or variant thereof disclosed herein. The bacterial strain disclosed herein, or a progeny, mutant, or variant thereof, a fermentate produced from a strain disclosed herein progeny, mutant, or variant thereof used in step (a) may be the same or different than the bacterial strain disclosed herein, or a progeny, mutant, or variant thereof, a fermentate produced from a strain disclosed herein progeny, mutant, or variant thereof used in step (b). The bacterial strain disclosed herein, or a progeny, mutant, or variant thereof, a fermentate produced from a strain disclosed herein progeny, mutant, or variant thereof used in step (a) may be the same or different than the bacterial strain disclosed herein, or a progeny, mutant, or variant thereof, a fermentate produced from a strain disclosed herein progeny, mutant, or variant thereof, used in step (c). The bacterial strain disclosed herein, or a progeny, mutant, or variant thereof, a fermentate produced from a strain disclosed herein progeny, mutant, or variant thereof used in step (b) may be the same or different than the bacterial strain disclosed herein, or a progeny, mutant, or variant thereof, a fermentate produced from a strain disclosed herein progeny, mutant, or variant thereof used in step (c).

In one embodiment, wild plant species and plant cultivars, or those obtained by conventional biological breeding, such as crossing or protoplast fusion, and parts thereof, can be treated with a bacterial strain disclosed herein, or a progeny, mutant, or variant thereof, a fermentate produced from a strain disclosed herein progeny, mutant, or variant thereof disclosed herein. In another embodiment, transgenic plants and plant cultivars obtained by genetic engineering, and plant parts thereof, are treated with a bacterial strain disclosed herein, or a progeny, mutant, or variant thereof, a fermentate produced from a strain disclosed herein progeny, mutant, or variant thereof disclosed herein.

In another embodiment, plants or plant cultivars (obtained by plant biotechnology methods such as genetic engineering or editing) that may be treated according to the strains, compositions and methods disclosed herein are herbicide-tolerant plants, i.e. plants made tolerant to one or more given herbicides. Such plants can be obtained either by genetic modification, or by selection of plants containing a mutation imparting such herbicide tolerance. Herbicide-resistant plants are for example glyphosate-tolerant plants, i.e. plants made tolerant to the herbicide glyphosate or salts thereof. Plants can be made tolerant to glyphosate through different means. For example, glyphosate-tolerant plants can be obtained by transforming the plant with a gene encoding the enzyme 5-enolpyruvylshilcimate-3-phosphate synthase (EPSPS).

Plants or plant cultivars (obtained by plant biotechnology methods such as genetic engineering or editing) that may also be treated are insect-resistant genetically modified plants, i.e. plants made resistant to attack by certain target insects. Such plants can be obtained by genetic transformation, or by selection of plants containing a mutation imparting such insect resistance.

In another embodiment, plants or plant cultivars (obtained by plant biotechnology methods such as genetic engineering) that may be treated according to the disclosure are tolerant to abiotic stresses. Such plants can be obtained by genetic transformation, or by selection of plants containing a mutation imparting such stress resistance.

In another embodiment, plants or plant cultivars (obtained by plant biotechnology methods such as genetic engineering or editing) that may be treated according to the disclosure are conventionally bred, by mutagenesis, or genetically engineered to contain a combination or stack of valuable traits, including but not limited to, herbicide tolerance, insect resistance, and abiotic stress tolerance.

The embodiments disclosed herein also apply to plant varieties which will be developed, or marketed, in the future and which have these genetic traits or traits to be developed in the future.

As used herein, applying a bacterial strain disclosed herein, or a progeny, mutant, or variant thereof, a fermentate produced from a strain disclosed herein progeny, mutant, or variant thereof to a seed, a plant, or plant part includes contacting the seed, plant, or plant part directly and/or indirectly with the bacterial strain disclosed herein, or a progeny, mutant, or variant thereof, a fermentate produced from a strain disclosed herein progeny, mutant, or variant thereof. In one embodiment, a bacterial strain disclosed herein, or a progeny, mutant, or variant thereof, a fermentate produced from a strain disclosed herein progeny, mutant, or variant thereof may be directly applied as a spray, a rinse, or a powder, or any combination thereof.

As used herein, a spray refers to a mist of liquid particles that contain a bacterial strain disclosed herein, or a progeny, mutant, or variant thereof, a fermentate produced from a strain disclosed herein progeny, mutant, or variant thereof of the present disclosure. In one embodiment, a spray may be applied to a plant or plant part while a plant or plant part is being grown. In another aspect, a spray may be applied to a plant or plant part while a plant or plant part is being fertilized. In another aspect, a spray may be applied to a plant or plant part while a plant or plant part is being harvested. In another aspect, a spray may be applied to a plant or plant part after a plant or plant part has been harvested. In another aspect, a spray may be applied to a plant or plant part while a plant or plant part is being processed. In another aspect, a spray may be applied to a plant or plant part while a plant or plant part is being packaged. In another aspect, a spray may be applied to a plant or plant part while a plant or plant part is being stored.

In another embodiment, a bacterial strain disclosed herein, or a progeny, mutant, or variant thereof, a fermentate produced from a strain disclosed herein progeny, mutant, or variant thereof disclosed herein may be applied directly to a plant or plant part as a rinse. As used herein, a rinse is a liquid containing a bacterial strain disclosed herein, or a progeny, mutant, or variant thereof, a fermentate produced from a strain disclosed herein progeny, mutant, or variant thereof disclosed herein. Such a rinse may be poured over a plant or plant part. A plant or plant part may also be immersed or submerged in the rinse, then removed and allowed to dry.

In another embodiment, a bacterial strain disclosed herein, or a progeny, mutant, or variant thereof, a fermentate produced from a strain disclosed herein progeny, mutant, or variant thereof may be applied to a plant or plant part and may cover 50% of the surface area of a plant material. In another embodiment, a bacterial strain disclosed herein, or a progeny, mutant, or variant thereof, a fermentate produced from a strain disclosed herein progeny, mutant, or variant thereof may be applied to a plant or plant part and may cover a percentage of the surface area of a plant material selected from the group consisting of: from 50% to about 95%, from 60% to about 95%, from 70% to about 95%, from 80% to about 95%, and from 90% to about 95%.

In another aspect, a bacterial strain disclosed herein, or a progeny, mutant, or variant thereof, a fermentate produced from a strain disclosed herein progeny, mutant, or variant thereof may cover from about 20% to about 30%, from about 30% to about 40%, from about 40% to about 50%, from about 50% to about 60%, from about 60% to about 70%, from about 70% to about 80%, from about 80% to about 90%, from about 90% to about 95%, from about 95% to about 98%, from about 98% to about 99% or 100% of the surface area of a plant or plant part.

In another aspect, a bacterial strain disclosed herein, or a progeny, mutant, or variant thereof, a fermentate produced from a strain disclosed herein progeny, mutant, or variant thereof disclosed herein may be applied directly to a plant or plant part as a powder. As used herein, a powder is a dry or nearly dry bulk solid composed of a large number of very fine particles that may flow freely when shaken or tilted. A dry or nearly dry powder composition disclosed herein preferably contains a low percentage of water, such as, for example, in various aspects, less than 5%, less than 2.5%, or less than 1% by weight.

In another aspect, a composition can be applied indirectly to the plant or plant part. For example, a plant or plant part having a bacterial strain disclosed herein, or a progeny, mutant, or variant thereof, a fermentate produced from a strain disclosed herein progeny, mutant, or variant thereof already applied may be touching a second plant or plant part so that a bacterial strain disclosed herein, or a progeny, mutant, or variant thereof, a fermentate produced from a strain disclosed herein progeny, mutant, or variant thereof rubs off on a second plant or plant part. In a further aspect, a bacterial strain disclosed herein, or a progeny, mutant, or variant thereof, a fermentate produced from a strain disclosed herein progeny, mutant, or variant thereof may be applied using an applicator. In various aspects, an applicator may include, but is not limited to, a syringe, a sponge, a paper towel, or a cloth, or any combination thereof.

A contacting step may occur while a plant material is being grown, while a plant or plant part is being fertilized, while a plant or plant part is being harvested, after a plant or plant part has been harvested, while a plant or plant part is being processed, while a plant or plant part is being packaged, or while a plant or plant part is being stored in warehouse or on the shelf of a store.

In another embodiment, a bacterial strain disclosed herein, or a progeny, mutant, or variant thereof, a fermentate produced from a strain disclosed herein progeny, mutant, or variant thereof disclosed herein may be a colloidal dispersion. A colloidal dispersion is a type of chemical mixture where one substance is dispersed evenly throughout another. Particles of the dispersed substance are only suspended in the mixture, unlike a solution, where they are completely dissolved within. This occurs because the particles in a colloidal dispersion are larger than in a solution—small enough to be dispersed evenly and maintain a homogenous appearance, but large enough to scatter light and not dissolve. Colloidal dispersions are an intermediate between homogeneous and heterogeneous mixtures and are sometimes classified as either “homogeneous” or “heterogeneous” based upon their appearance.

In one embodiment, the bacterial strain disclosed herein, or a progeny, mutant, or variant thereof, a fermentate produced from a strain disclosed herein progeny, mutant, or variant thereof compositions and methods disclosed herein are suitable for use with a seed. In another embodiment, the the bacterial strain disclosed herein, or a progeny, mutant, or variant thereof, a fermentate produced from a strain disclosed herein progeny, mutant, or variant thereof compositions and methods disclosed herein are suitable for use with a seed of one or more of any of the plants recited previously.

In still another embodiment, the bacterial strain disclosed herein, or a progeny, mutant, or variant thereof, a fermentate produced from a strain disclosed herein progeny, mutant, or variant thereof compositions and methods disclosed herein can be used to treat transgenic or genetically modified or edited seed. A transgenic seed refers to the seed of plants containing at least one heterologous gene that allows the expression of a polypeptide or protein not naturally found in the plant. The heterologous gene in transgenic seed can originate, for example, from microorganisms of the species Bacillus, Rhizobium, Pseudomonas, Serratia, Trichoderma, Cavibacter, Glomus or Gliocladium.

In one embodiment, the seed is treated in a state in which it is sufficiently stable so that the treatment does not cause any damage. In general, treatment of the seed may take place at any point in time between harvesting and sowing. In one embodiment, the seed used is separated from the plant and freed from cobs, shells, stalks, coats, hairs or the flesh of the fruits. Thus, it is possible to use, for example, seed which has been harvested, cleaned and dried. Alternatively, it is also possible to use seed which, after drying, has been treated, for example, with water and then dried again.

In one embodiment, seed is treated with a bacterial strain disclosed herein, or a progeny, mutant, or variant thereof, a fermentate produced from a strain disclosed herein progeny, mutant, or variant thereof compositions and methods disclosed herein in such a way that the germination of the seed is not adversely affected, or that the resulting plant is not damaged.

In one embodiment, a bacterial strain disclosed herein, or a progeny, mutant, or variant thereof, a fermentate produced from a strain disclosed herein progeny, mutant, or variant thereof compositions disclosed herein may be applied directly to the seed. For example, the bacterial strain disclosed herein, or a progeny, mutant, or variant thereof, fermentate produced from a strain disclosed herein progeny, mutant, or variant thereof compositions disclosed herein may be applied without additional components and without having been diluted.

In another embodiment, a bacterial strain disclosed herein, or a progeny, mutant, or variant thereof, a fermentate produced from a strain disclosed herein progeny, mutant, or variant thereof disclosed herein may be applied to the seed in the form of a suitable formulation. Suitable formulations and methods for the treatment of seed are known to the person skilled in the art and are described, for example, in the following documents: U.S. Pat. Nos. 4,272,417 A, 4,245,432 A, 4,808,430 A, 5,876,739 A, US 2003/0176428 A1, WO 2002/080675 A1, WO 2002/028186 A2.

A bacterial strain disclosed herein, or a progeny, mutant, or variant thereof, a fermentate produced from a strain disclosed herein progeny, mutant, or variant thereof disclosed herein can be converted into customary seed dressing formulations, such as solutions, emulsions, suspensions, powders, foams, slurries or other coating materials for seed, and also ULV formulations. These formulations are prepared in a known manner by mixing A bacterial strain disclosed herein, or a progeny, mutant, or variant thereof, a fermentate produced from a strain disclosed herein progeny, mutant, or variant thereof disclosed herein with customary additives, such as, for example, customary extenders and also solvents or diluents, colorants, wetting agents, dispersants, emulsifiers, defoamers, preservatives, secondary thickeners, adhesives, gibberellins and water as well.

In another embodiment, suitable colorants that may be present in the seed dressing formulations include all colorants customary for such purposes. Use may be made both of pigments, of sparing solubility in water, and of dyes, which are soluble in water. Examples that may be mentioned include the colorants known under the designations Rhodamine B, C.I. Pigment Red 112, and C. I. Solvent Red 1.

In another embodiment, suitable wetting agents that may be present in the seed dressing formulations include all substances that promote wetting and are customary in the formulation of active agrochemical substances. With preference it is possible to use alkylnaphthalene-sulphonates, such as diisopropyl- or diisobutylnaphthalene-sulphonates.

In still another embodiment, suitable dispersants and/or emulsifiers that may be present in the seed dressing formulations include all nonionic, anionic, and cationic dispersants that are customary in the formulation of active agrochemical substances. In one embodiment, nonionic or anionic dispersants or mixtures of nonionic or anionic dispersants can be used. In one embodiment, nonionic dispersants include but are not limited to ethylene oxide-propylene oxide block polymers, alkylphenol polyglycol ethers, and tristyrylphenol polyglycol ethers, and their phosphated or sulphated derivatives.

In still another embodiment, defoamers that may be present in the seed dressing formulations to be used include all foam-inhibiting compounds that are customary in the formulation of agrochemically active compounds including but not limited to silicone defoamers, magnesium stearate, silicone emulsions, long-chain alcohols, fatty acids and their salts and also organofluorine compounds and mixtures thereof.

In still another embodiment, secondary thickeners that may be present in the seed dressing formulations include all compounds which can be used for such purposes in agrochemical compositions, including but not limited to cellulose derivatives, acrylic acid derivatives, polysaccharides, such as xanthan gum or Veegum, modified clays, phyllosilicates, such as attapulgite and bentonite, and also finely divided silicic acids.

Suitable adhesives that may be present in the seed dressing formulations may include all customary binders which can be used in seed dressings. Polyvinylpyrrolidone, polyvinyl acetate, polyvinyl alcohol and tylose may be mentioned as being preferred.

In yet another embodiment, seed dressing formulations may be used directly or after dilution with water beforehand to treat seed of any of a very wide variety of types. The seed dressing formulations or their dilute preparations may also be used to dress seed of transgenic plants. In this context, synergistic effects may also arise in interaction with the substances formed by expression.

Suitable mixing equipment for treating seed with the seed dressing formulations or the preparations prepared from them by adding water includes all mixing equipment that can commonly be used for dressing. The specific procedure adopted when dressing comprises introducing the seed into a mixer, adding the particular desired amount of seed dressing formulation, either as it is or following dilution with water beforehand, and carrying out mixing until the formulation is uniformly distributed on the seed. Optionally, a drying operation follows.

In various embodiments, a bacterial strain disclosed herein, or a progeny, mutant, or variant thereof, a fermentate produced from a strain disclosed herein progeny, mutant, or variant thereof, can be added to the plant, plant part, and/or seed at a rate of about 1×10² to 1×10¹³ colony forming units (cfu) per seed, including about 1×10³ cfu/seed, or about 1×10⁴ cfu/seed, 1×10⁵ cfu/seed, or about 1×10⁶ cfu/seed, or about 1×10⁷ cfu/seed, or about 1×10⁸ cfu/seed, or about 1×10⁹ cfu/seed, or about 1×10¹⁰ cfu/seed, or about 1×10¹¹ cfu/seed, or about 1×10¹² cfu/seed, or about 1×10¹³ cfu/seed including about 1×10³ to 1×10⁸ cfu/seed about 1×10³ to 1×10⁷ cfu/seed, about 1×10³ to 1×10⁵ cfu/seed, about 1×10³ to 1×10⁶ cfu/seed, about 1×10³ to 1×10⁴ cfu/seed, about 1×10³ to 1×10⁵ cfu/seed, about 1×10³ to 1×10¹⁰ cfu/seed, about 1×10³ to 1×10¹¹ cfu/seed, about 1×10³ to 1×10¹² cfu/seed, about 1×10³ to 1×10¹³ cfu/seed, about 1×10⁴ to 1×10⁸ cfu/seed about 1×10⁴ to 1×10⁷ cfu/seed, about 1×10⁴ to 1×10⁵ cfu/seed, about 1×10⁴ to 1×10⁶ cfu/seed, about 1×10⁴ to 1×10⁹ cfu/seed, about 1×10⁴ to 1×10¹⁰ cfu/seed, about 1×10¹¹ to 1×10⁹ cfu/seed, about 1×10⁴ to 1×10¹² cfu/seed about 1×10⁴ to 1×10¹³ cfu/seed, about 1×10⁵ to 1×10⁷ cfu/per seed, about 1×10⁵ to 1×10⁶ cfu/per seed, about 1×10⁵ to 1×10⁸ cfu/per seed, about 1×10⁵ to 1×10⁹ cfu/per seed, about 1×10⁵ to 1×10¹⁰ cfu/per seed, about 1×10⁵ to 1×10¹¹ cfu/per seed, about 1×10⁵ to 1×10¹² cfu/per seed, about 1×10⁵ to 1×10¹³ cfu/per seed, about 1×10⁶ to 1×10¹² cfu/per seed, about 1×10⁶ to 1×10⁷ cfu/per seed, about 1×10⁶ to 1×10⁹ cfu/per seed, about 1×10⁶ to 1×10¹⁰ cfu/per seed, about 1×10⁶ to 1×10¹¹ cfu/per seed, about 1×10⁶ to 1×10¹² cfu/per seed, about 1×10⁶ to 1×10¹³ cfu/per seed, about 1×10⁷ to 1×10⁸ cfu/per seed, about 1×10⁷ to 1×10⁹ cfu/per seed, about 1×10⁷ to 1×10¹⁰ cfu/per seed, about 1×10⁷ to 1×10¹¹ cfu/per seed, about 1×10⁷ to 1×10¹² cfu/per seed, about 1×10⁷ to 1×10¹³ cfu/per seed, about 1×10⁸ to 1×10⁹ cfu/per seed, about 1×10⁸ to 1×10¹⁰ cfu/per seed, about 1×10⁸ to 1×10¹¹ cfu/per seed, about 1×10⁹ to 1×10¹² cfu/per seed, about 1×10⁸ to 1×10¹³ cfu/per seed, about 1×10⁹ to 1×10¹⁰ cfu/per seed, about 1×10⁹ to 1×10¹¹ cfu/per seed, about 1×10⁹ to 1×10¹² cfu/per seed, about 1×10⁹ to 1×10¹³ cfu/per seed, about 1×10¹⁰ to 1×10¹¹ cfu/per seed, about 1×10¹⁰ to 1×10¹² cfu/per seed, about 1×10¹⁰ to 1×10¹³ cfu/per seed, about 1×10¹¹¹ to 1×10¹² cfu/per seed, about 1×10¹¹ to 1×10¹³ cfu/per seed, and about 1×10¹² to 1×10¹³ cfu/per seed. As used herein, the tem “colony forming unit” or “cfu” is a unit capable of growing and producing a colony of a microbial strain in favorable conditions.

In one embodiment, a bacterial strain disclosed herein, or a progeny, mutant, or variant thereof, a fermentate produced from a strain disclosed herein progeny, mutant, or variant thereof, may be formulated as a liquid seed treatment. A seed treatment may comprise at least one a bacterial strain disclosed herein, or a progeny, mutant, or variant thereof, a fermentate produced from a strain disclosed herein progeny, mutant, or variant thereof. The seeds are substantially uniformly coated with one or more layers of a bacterial strain disclosed herein, or a progeny, mutant, or variant thereof, a fermentate produced from a strain disclosed herein progeny, mutant, or variant thereof, using conventional methods of mixing, spraying or a combination thereof. Application is done using equipment that accurately, safely, and efficiently applies seed treatment products to seeds. Such equipment uses various types of coating technology such as rotary coaters, drum coaters, fluidized bed techniques, spouted beds, rotary mists or a combination thereof.

In one embodiment, the application is done via either a spinning “atomizer” disk or a spray nozzle that evenly distributes the seed treatment onto the seed as it moves through the spray pattern. In yet another embodiment, the seed is then mixed or tumbled for an additional period of time to achieve additional treatment distribution and drying. The seeds may be primed or unprimed before coating with a composition disclosed herein to increase the uniformity of germination and emergence. In an alternative embodiment, a dry powder composition can be metered onto the moving seed.

In still another embodiment, the seeds may be coated via a continuous or batch coating process. In a continuous coating process, continuous flow equipment simultaneously meters both the seed flow and the seed treatment products. A slide gate, cone and orifice, seed wheel, or weight device (belt or diverter) regulates seed flow. Once the seed flow rate through treating equipment is determined, the flow rate of the seed treatment is calibrated to the seed flow rate in order to deliver the desired dose to the seed as it flows through the seed treating equipment. Additionally, a computer system may monitor the seed input to the coating machine, thereby maintaining a constant flow of the appropriate amount of seed.

In a batch coating process, batch treating equipment weighs out a prescribed amount of seed and places the seed into a closed treating chamber or bowl where the corresponding of seed treatment is then applied. The seed and seed treatment are then mixed to achieve a substantially uniform coating on each seed. This batch is then dumped out of the treating chamber in preparation for the treatment of the next batch. With computer control systems, this batch process is automated enabling it to continuously repeat the batch treating process.

A variety of additives can be added to the seed treatments. Binders can be added and include those composed preferably of an adhesive polymer that can be natural or synthetic without phytotoxic effect on the seed to be coated. A variety of colorants may be employed, including organic chromophores classified as nitroso, nitro, azo, including monoazo, bisazo, and polyazo, diphenylmethane, triarylmethane, xanthene, methane, acridine, thiazole, thiazine, indamine, indophenol, azine, oxazine, anthraquinone, and phthalocyanine. Other additives that can be added include trace nutrients such as salts of iron, manganese, boron, copper, cobalt, molybdenum, and zinc. A polymer or other dust control agent can be applied to retain the treatment on the seed surface.

Other conventional seed treatment additives include, but are not limited to, coating agents, wetting agents, buffering agents, and polysaccharides. At least one agriculturally acceptable carrier can be added to the seed treatment formulation such as water, solids or dry powders. The dry powders can be derived from a variety of materials such as wood barks, calcium carbonate, gypsum, vermiculite, talc, humus, activated charcoal, and various phosphorous compounds.

In one embodiment, the seed coating can comprise of at least one filler, which is an organic or inorganic, natural or synthetic component with which a bacterial strain disclosed herein, or a progeny, mutant, or variant thereof, a fermentate produced from a strain disclosed herein progeny, mutant, or variant thereof described herein is combined to facilitate its application onto the seed. In one embodiment, the filler is an inert solid such as clays, natural or synthetic silicates, silica, resins, waxes, solid fertilizers (for example ammonium salts), natural soil minerals, such as kaolins, clays, talc, lime, quartz, attapulgite, montmorillonite, bentonite, or diatomaceous earths, or synthetic minerals, such as silica, alumina, or silicates, in particular aluminum or magnesium silicates.

In one embodiment, a bacterial strain disclosed herein, or a progeny, mutant, or variant thereof, a fermentate produced from a strain disclosed herein progeny, mutant, or variant thereof disclosed herein may be formulated by encapsulation technology to improve fungal spore stability and as a way to protect the fungal spores from seed applied fungicides. In one embodiment the encapsulation technology may comprise a bead polymer for timed release of fungal spores over time. In one embodiment, the encapsulation technology may comprise a zeolite material. In one embodiment, an encapsulated bacterial strain disclosed herein, or a progeny, mutant, or variant thereof, fermentate produced from a strain disclosed herein progeny, mutant, or variant thereof may be applied in a separate application of beads in-furrow to the seeds. In another embodiment, the encapsulated bacterial strain disclosed herein, or a progeny, mutant, or variant thereof, fermentate produced from a strain disclosed herein progeny, mutant, or variant thereof may be co-applied along with seeds simultaneously.

Insect resistance management (IRM) is the term used to describe practices aimed at reducing the potential for insect pests to become resistant to an insect management tactic. IRM maintenance of Bt (Bacillus thuringiensis) derived pesticidal proteins, other pesticidal proteins, a chemical, a biological agent, or other biologicals, is of great importance because of the threat insect resistance poses to the future use of pesticidal plant-incorporated protectants and insecticidal trait technology as a whole. Specific IRM strategies, such as the refuge strategy, mitigate insect resistance to specific insecticidal proteins produced in corn, soybean, cotton, and other crops. However, such strategies result in portions of crops being left susceptible to one or more pests in order to ensure that non-resistant insects develop and become available to mate with any resistant pests produced in protected crops. Accordingly, from a farmer/producer's perspective, it is highly desirable to have as small a refuge as possible and yet still manage insect resistance, in order that the greatest yield be obtained while still maintaining the efficacy of the pest control method used, whether Bt, a different pesticidal protein, chemical, biological agent or other biologicals, some other method, or combinations thereof.

Another strategy to reduce the need for refuge is the pyramiding of traits with different modes of action against a target insect pest. For example, Bt toxins that have different modes of action pyramided in one transgenic plant are able to have reduced refuge requirements due to reduced resistance risk. Different modes of action in a pyramid combination also extend the durability of each trait, as resistance is slower to develop to each trait.

Currently, the size, placement, and management of the refuge are often considered critical to the success of refuge strategies to mitigate insect resistance to the Bt/pesticidal trait produced in corn, cotton, soybean, and other crops. Because of the decrease in yield in refuge planting areas, some farmers choose to eschew the refuge requirements, and others do not follow the size and/or placement requirements. These issues result in either no refuge or a less effective refuge, and a corresponding risk of the increase in the development of resistance pests.

Accordingly, there remains a need for methods for managing pest resistance in a plot of pest resistant crop plants. It would be useful to provide an improved method for the protection of plants, especially corn or other crop plants, from feeding damage by pests. It would be particularly useful if such a method would reduce the required application rate of conventional chemical pesticides, and also if it would limit the number of separate field operations that were required for crop planting and cultivation. In addition, it would be useful to have a method of deploying a biocontrol agent that increases the durability of an insecticidal trait or increases the efficacy of many resistance management strategies.

One embodiment relates to a method of reducing or preventing the resistance of pests to a plant pesticidal composition comprising providing a plant protection composition, such as a Bt pesticidal protein, a transgenic pesticidal protein, other pesticidal proteins, chemical pesticides, or pesticidal biological entomopathogens, to a plant and/or plant part or a planted area or insecticidal trait and providing a bacterial strain disclosed herein, or a progeny, mutant, or variant thereof, a fermentate produced from a strain disclosed herein progeny, mutant, or variant thereof described herein to the plant and/or plant part or planted area. Another embodiment relates to a method of reducing or preventing the resistance to a plant insecticidal trait comprising providing or contacting a plant with a bacterial strain disclosed herein, or a progeny, mutant, or variant thereof, a fermentate produced from a strain disclosed herein progeny, mutant, or variant thereof described herein.

A further embodiment relates to a method of increasing the durability of plant pest compositions comprising providing a plant protection composition to a plant or planted area, and providing a bacterial strain disclosed herein, or a progeny, mutant, or variant thereof, a fermentate produced from a strain disclosed herein progeny, mutant, or variant thereof described herein to the plant or planted area, wherein the bacterial strain disclosed herein, or a progeny, mutant, or variant thereof, fermentate produced from a strain disclosed herein progeny, mutant, or variant thereof described herein have a different mode of action than the plant protection composition.

In a still further embodiment, the refuge required may be reduced or eliminated by the presence of a bacterial strain disclosed herein, or a progeny, mutant, or variant thereof, a fermentate produced from a strain disclosed herein progeny, mutant, or variant thereof described herein applied to the non-refuge plants. In another embodiment, the refuge may include a bacterial strain disclosed herein, or a progeny, mutant, or variant thereof, a fermentate produced from a strain disclosed herein progeny, mutant, or variant thereof described herein as a spray, bait, or as a different mode of action.

In one embodiment, a composition comprises a bacterial strain disclosed herein, or a progeny, mutant, or variant thereof, a fermentate produced from a strain disclosed herein progeny, mutant, or variant thereof disclosed herein and a non-Bt insecticidal trait increases resistance to a pathogen, pest, or insect. In another embodiment, the non-Bt insecticidal trait comprises a plant-derived insecticidal protein, a bacterial/archeal-derived insecticidal protein not from a Bt (such as a Pseudomonas insecticidal protein), an animal-derived insecticidal protein, or a silencing element. In another embodiment, a composition comprising a bacterial strain disclosed herein, or a progeny, mutant, or variant thereof, a fermentate produced from a strain disclosed herein progeny, mutant, or variant thereof disclosed herein and a non-Bt insecticidal trait increases durability of the non-Bt insecticidal trait. In another embodiment, the non-Bt insecticidal trait comprises a PIP-72 polypeptide of PCT Serial Number PCT/US14/55128. In another embodiment, the non-Bt insecticidal trait comprises a polynucleotide silencing elements targeting RyanR (DvSSJ) (US Patent Application publication 2014/0275208). In another embodiment, the non-Bt insecticidal trait comprises a polynucleotide silencing elements targeting RyanR (DvSSJ) (US Patent Application publication 2014/0275208, herein incorporated by reference in its entirety) and a PIP-72 polypeptide of PCT Serial Number PCT/US14/55128, herein incorporated by reference in its entirety.

In another embodiment, a composition comprising a bacterial strain disclosed herein, or a progeny, mutant, or variant thereof, a fermentate produced from a strain disclosed herein progeny, mutant, or variant thereof disclosed herein and a fungal entomopathogen disclosed in U.S. Pat. No. 9,993,006, herein incorporated by reference in its entirety.

In some embodiments, a composition comprises a bacterial strain disclosed herein, or a progeny, mutant, or variant thereof, a fermentate produced from a strain disclosed herein progeny, mutant, or variant thereof disclosed herein and a Bt insecticidal trait that increases resistance to a pathogen, pest, or insect. A Bt insecticidal trait may have activity to Coleopteran, Lepidopteran, or Hemipteran plant pests. The compositions disclosed herein may provide to a plant or plant part additive or synergistic resistance to a pathogen, pest, or insect plant in combination with a Bt insecticidal trait. In one embodiment, a composition comprises a bacterial strain disclosed herein, or a progeny, mutant, or variant thereof, a fermentate produced from a strain disclosed herein progeny, mutant, or variant thereof disclosed herein and a Bt insecticidal trait, wherein the Bt insecticidal trait comprises a Cry3B toxin disclosed in U.S. Pat. Nos. 8,101,826, 6,551,962, 6,586,365, 6,593,273, and PCT Publication WO 2000/011185, a mCry3B toxin disclosed in U.S. Pat. Nos. 8,269,069, and 8,513,492, a mCry3A toxin disclosed in U.S. Pat. Nos. 8,269,069, 7,276,583 and 8,759,620, or a Cry34/35 toxin disclosed in U.S. Pat. Nos. 7,309,785, 7,524,810, 7,985,893, 7,939,651 and 6,548,291, and transgenic events containing these Bt insecticidal toxins and other Coleopteran active Bt insecticidal traits for example, event MON863 disclosed in U.S. Pat. No. 7,705,216, event MIR604 disclosed in U.S. Pat. No. 8,884,102, event 5307 disclosed in U.S. Pat. No. 9,133,474, event DAS-59122 disclosed in U.S. Pat. No. 7,875,429, event DP-4114 disclosed in U.S. Pat. No. 8,575,434, event MON 87411 disclosed in US Published Patent Application Number 2013/0340111, and event MON88017 disclosed in U.S. Pat. No. 8,686,230 all of which are incorporated herein by reference. All publications, patents and patent applications mentioned in the specification indicate the level of those skilled in the art to which this disclosure pertains. All publications, patents and patent applications are incorporated by reference to the same extent as if each individual publication, patent or patent application was specifically and individually indicated to be incorporated by reference.

### IPD126 Proteins and Variants and Fragments Thereof

IPD126 polypeptides are encompassed by the disclosure as set forth in SEQ ID NOs: 19-36. “IPD126 polypeptide,” and “IPD126 protein” as used herein interchangeably refers to a polypeptide(s) having insecticidal activity including but not limited to insecticidal activity against one or more insect pests of the Lepidoptera, Hemiptera, and/or Coleoptera orders. A variety of IPD126 polypeptides are contemplated.

“Sufficiently identical” is used herein to refer to an amino acid sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity. In one embodiment the IPD126 polypeptide has at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to any one of SEQ ID NOs: 19-36. The term “about” when used herein in context with percent sequence identity means+/−1.0%.

A “recombinant protein” is used herein to refer to a protein that is no longer in its natural environment, for example in vitro or in a recombinant bacterial or plant host cell.

“Fragments” or “biologically active portions” include polypeptide fragments comprising amino acid sequences sufficiently identical to an IPD126 polypeptide and that exhibit insecticidal activity. “Fragments” or “biologically active portions” of IPD126 polypeptides includes fragments comprising amino acid sequences sufficiently identical to the amino acid sequence set forth in any one of SEQ ID NOs: 19-36 wherein the IPD126 polypeptide has insecticidal activity. Such biologically active portions can be prepared by recombinant techniques and evaluated for insecticidal activity.

“Variants” as used herein refers to proteins or polypeptides having an amino acid sequence that is at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or greater identical to the parental amino acid sequence.

In some embodiments an IPD126 polypeptide comprises an amino acid sequence having at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity to the full length or a fragment of the amino acid sequence of any one of SEQ ID NOs: 19-36, wherein the IPD126 polypeptide has insecticidal activity.

In some embodiments an IPD126 polypeptide comprises an amino acid sequence of any one or more of SEQ ID NOS: 19-36 having 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or more amino acid substitutions compared to the amino acid at the corresponding position of any one or more of the respective SEQ ID NOS: 19-36.

Methods for such manipulations are generally known in the art. For example, amino acid sequence variants of an IPD126 polypeptide can be prepared by mutations in the DNA. This may also be accomplished by one of several forms of mutagenesis, such as for example site-specific double strand break technology, and/or in directed evolution. In some aspects, the changes encoded in the amino acid sequence will not substantially affect the function of the protein. Such variants will possess a desired pesticidal activity. However, it is understood that the ability of an IPD126 polypeptide to confer pesticidal activity or other polypeptide physical property may be improved or altered by the use of such techniques upon the compositions of this disclosure.

Conservative amino acid substitutions may be made at one or more predicted nonessential amino acid residues. A “nonessential” amino acid residue is a residue that can be altered from the wild-type sequence of an IPD126 polypeptide without altering the biological activity. A “conservative amino acid substitution” is one in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); polar, negatively charged residues and their amides (e.g., aspartic acid, asparagine, glutamic, acid, glutamine; uncharged polar side chains (e.g., glycine, asparagine, glutamine, serine, threonine, tyrosine, cysteine); small aliphatic, nonpolar or slightly polar residues (e.g., Alanine, serine, threonine, proline, glycine); nonpolar side chains (e.g., alanine, valine, leucine, isoleucine, proline, phenylalanine, methionine, tryptophan); large aliphatic, nonpolar residues (e.g., methionine, leucine, isoleucine, valine, cystine); beta-branched side chains (e.g., threonine, valine, isoleucine); aromatic side chains (e.g., tyrosine, phenylalanine, tryptophan, histidine); large aromatic side chains (e.g., tyrosine, phenylalanine, tryptophan).

Amino acid substitutions may be made in nonconserved regions that retain function. In general, such substitutions would not be made for conserved amino acid residues or for amino acid residues residing within a conserved motif, where such residues are essential for protein activity. Examples of residues that are conserved and that may be essential for protein activity include, for example, residues that are identical between all proteins contained in an alignment of similar or related toxins to the sequences of the embodiments (e.g., residues that are identical in an alignment of homologous proteins). Examples of residues that are conserved but that may allow conservative amino acid substitutions and still retain activity include, for example, residues that have only conservative substitutions between all proteins contained in an alignment of similar or related toxins to the sequences of the embodiments (e.g., residues that have only conservative substitutions between all proteins contained in the alignment homologous proteins). However, one of skill in the art would understand that functional variants may have minor conserved or nonconserved alterations in the conserved residues.

Variant nucleotide and amino acid sequences of the disclosure also encompass sequences derived from mutagenic and recombinogenic procedures such as DNA shuffling. With such a procedure, one or more different IPD126 polypeptide coding regions can be used to create a new IPD126 polypeptide possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between a pesticidal gene and other known pesticidal genes to obtain a new gene coding for a protein with an improved property of interest, such as an increased insecticidal activity. Strategies for such DNA shuffling are known in the art. See, for example, Stemmer, (1994) Proc. Natl. Acad. Sci. USA 91:10747-10751; Stemmer, (1994) Nature 370:389-391; and U.S. Pat. Nos. 5,605,793 and 5,837,458.

In some embodiments, chimeric polypeptides are provided comprising regions of at least two different IPD126 polypeptides selected from any one or more of SEQ ID NOS: 19-36.

### Nucleic Acid Molecules, and Variants and Fragments Thereof

Isolated or recombinant nucleic acid molecules comprising nucleic acid sequences encoding IPD126 polypeptides or biologically active portions thereof, as well as nucleic acid molecules sufficient for use as hybridization probes to identify nucleic acid molecules encoding proteins with regions of sequence homology are provided. As used herein, the term “nucleic acid molecule” refers to DNA molecules (e.g., recombinant DNA, cDNA, genomic DNA, plastid DNA, mitochondrial DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule (or DNA) is used herein to refer to a nucleic acid sequence (or DNA) that is no longer in its natural environment, for example in vitro. A “recombinant” nucleic acid molecule (or DNA) is used herein to refer to a nucleic acid sequence (or DNA) that is in a recombinant bacterial or plant host cell. In some embodiments, an “isolated” or “recombinant” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the disclosure, “isolated” or “recombinant” when used to refer to nucleic acid molecules excludes isolated chromosomes. For example, in various embodiments, the recombinant nucleic acid molecules encoding IPD126 polypeptides can contain less than about 5 kb, 4 kb, 3 kb, 2 kb, 1 kb, 0.5 kb or 0.1 kb of nucleic acid sequences that naturally flank the nucleic acid molecule in genomic DNA of the cell from which the nucleic acid is derived.

In some embodiments an isolated nucleic acid molecule encoding IPD126 polypeptides has one or more change in the nucleic acid sequence compared to the native or genomic nucleic acid sequence. In some embodiments the change in the native or genomic nucleic acid sequence includes but is not limited to: changes in the nucleic acid sequence due to the degeneracy of the genetic code; changes in the nucleic acid sequence due to the amino acid substitution, insertion, deletion and/or addition compared to the native or genomic sequence; removal of one or more intron; deletion of one or more upstream or downstream regulatory regions; and deletion of the 5′ and/or 3′ untranslated region associated with the genomic nucleic acid sequence. In some embodiments the nucleic acid molecule encoding an IPD126 polypeptide is a non-genomic sequence.

A variety of polynucleotides that encode IPD126 polypeptides or related proteins are contemplated. Such polynucleotides are useful for production of IPD126 polypeptides in host cells when operably linked to a suitable promoter, transcription termination and/or polyadenylation sequences. Such polynucleotides are also useful as probes for isolating homologous or substantially homologous polynucleotides that encode IPD126 polypeptides or related proteins.

The polynucleotides of any one or more of SEQ ID NOS: 1-18, can be used to express IPD126 polypeptides in recombinant bacterial hosts that include but are not limited to Agrobacterium, Bacillus, Escherichia, Salmonella, Lysinibacillus, Acetobacter, Pseudomonas and Rhizobium bacterial host cells. The polynucleotides are also useful as probes for isolating homologous or substantially homologous polynucleotides encoding IPD126 polypeptides or related proteins. Such probes can be used to identify homologous or substantially homologous polynucleotides, or portions thereof, derived from Bacillus thurengiensis.

Polynucleotides encoding IPD126 polypeptides can also be synthesized de novo from an IPD126 polypeptide sequence. The sequence of the polynucleotide gene can be deduced from an IPD126 polypeptide sequence through use of the genetic code. Computer programs such as “BackTranslate” (GCG^{T}M Package, Acclerys, Inc. San Diego, Calif.) can be used to convert a peptide sequence to the corresponding nucleotide sequence encoding the peptide. Examples of IPD126 polypeptide sequences that can be used to obtain corresponding nucleotide encoding sequences include, but are not limited to the IPD126 polypeptides of SEQ ID NOS: 19-36. Furthermore, synthetic IPD126 polynucleotide sequences of the disclosure can be designed so that they will be expressed in plants.

In some embodiments the nucleic acid molecule encoding a IPD126 polypeptide is a polynucleotide having the sequence set forth in any one of SEQ ID NOS: 1-18, and variants, fragments and complements thereof. “Complement” is used herein to refer to a nucleic acid sequence that is sufficiently complementary to a given nucleic acid sequence such that it can hybridize to the given nucleic acid sequence to thereby form a stable duplex. “Polynucleotide sequence variants” is used herein to refer to a nucleic acid sequence that except for the degeneracy of the genetic code encodes the same polypeptide.

In some embodiments the nucleic acid molecule encoding the IPD126 polypeptide is a non-genomic nucleic acid sequence. As used herein a “non-genomic nucleic acid sequence” or “non-genomic nucleic acid molecule” or “non-genomic polynucleotide” refers to a nucleic acid molecule that has one or more change in the nucleic acid sequence compared to a native or genomic nucleic acid sequence. In some embodiments the change to a native or genomic nucleic acid molecule includes but is not limited to: changes in the nucleic acid sequence due to the degeneracy of the genetic code; optimization of the nucleic acid sequence for expression in plants; changes in the nucleic acid sequence to introduce at least one amino acid substitution, insertion, deletion and/or addition compared to the native or genomic sequence; removal of one or more intron associated with the genomic nucleic acid sequence; insertion of one or more heterologous introns; deletion of one or more upstream or downstream regulatory regions associated with the genomic nucleic acid sequence; insertion of one or more heterologous upstream or downstream regulatory regions; deletion of the 5′ and/or 3′ untranslated region associated with the genomic nucleic acid sequence; insertion of a heterologous 5′ and/or 3′ untranslated region; and modification of a polyadenylation site. In some embodiments the non-genomic nucleic acid molecule is a synthetic nucleic acid sequence.

In some embodiments the nucleic acid molecule encoding a IPD126 polypeptide disclosed herein is a non-genomic polynucleotide having a nucleotide sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity, to the nucleic acid sequence of any one of SEQ ID NOS: 1-18, wherein the IPD126 polypeptide has insecticidal activity.

In some embodiments the nucleic acid molecule encodes an IPD126 polypeptide variant comprising one or more amino acid substitutions to the amino acid sequence of any one of SEQ ID NOS: 19-36.

Nucleic acid molecules that are fragments of these nucleic acid sequences encoding IPD126 polypeptides are also encompassed by the embodiments. “nucleotide fragment” as used herein refers to a portion of the nucleic acid sequence encoding an IPD126 polypeptide. A nucleotide fragment of a nucleic acid sequence may encode a biologically active portion of an IPD126 polypeptide or it may be a fragment that can be used as a hybridization probe or PCR primer using methods disclosed below. Nucleic acid molecules that are fragments of a nucleic acid sequence encoding an IPD126 polypeptide comprise at least about 150, 180, 210, 240, 270, 300, 330, 360, 400, 450, or 500 contiguous nucleotides or up to the number of nucleotides present in a full-length nucleic acid sequence encoding an IPD126 polypeptide disclosed herein, depending upon the intended use. “Contiguous nucleotides” is used herein to refer to nucleotide residues that are immediately adjacent to one another. Fragments of the nucleic acid sequences of the embodiments will encode protein fragments that retain the biological activity of the IPD126 polypeptide and, hence, retain insecticidal activity. “Retains insecticidal activity” is used herein to refer to a polypeptide having at least about 10%, at least about 30%, at least about 50%, at least about 70%, 80%, 90%, 95% or higher of the insecticidal activity of any one of the full-length IPD126 polypeptides set forth in SEQ ID NOS: 19-36. In some embodiments, the insecticidal activity is against a Lepidopteran species. In one embodiment, the insecticidal activity is against a Coleopteran species. In some embodiments, the insecticidal activity is against one or more insect pests of the corn rootworm complex: western corn rootworm, Diabrotica virgifera; northern corn rootworm, D. barberi: Southern corn rootworm or spotted cucumber beetle; Diabrotica undecimpunctata howardi, Diabrotica speciosa, and the Mexican corn rootworm, D. virgifera zeae. In one embodiment, the insecticidal activity is against a Diabrotica species.

In some embodiments the IPD126 polypeptide is encoded by a nucleic acid sequence sufficiently homologous to any one of the nucleic acid sequences of SEQ ID NOS: 1-18.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence x 100).

In some embodiments an IPD126 polynucleotide encodes an IPD126 polypeptide comprising an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or greater sequence identity across the entire length of the amino acid sequence of any one of SEQ ID NOS: 19-36.

In some embodiments polynucleotides are provided encoding chimeric polypeptides comprising regions of at least two different IPD126 polypeptides of the disclosure.

The embodiments also encompass nucleic acid molecules encoding IPD126 polypeptide variants. “Variants” of the IPD126 polypeptide encoding nucleic acid sequences include those sequences that encode the IPD126 polypeptides disclosed herein but that differ conservatively because of the degeneracy of the genetic code as well as those that are sufficiently identical as discussed above. Naturally occurring allelic variants can be identified with the use of well-known molecular biology techniques, such as polymerase chain reaction (PCR) and hybridization techniques as outlined below. Variant nucleic acid sequences also include synthetically derived nucleic acid sequences that have been generated, for example, by using site-directed mutagenesis but which still encode the IPD126 polypeptides disclosed as discussed below.

The present disclosure provides isolated or recombinant polynucleotides that encode any of the IPD126 polypeptides disclosed herein. Those having ordinary skill in the art will readily appreciate that due to the degeneracy of the genetic code, a multitude of nucleotide sequences encoding IPD126 polypeptides of the present disclosure exist.

The skilled artisan will further appreciate that changes can be introduced by mutation of the nucleic acid sequences thereby leading to changes in the amino acid sequence of the encoded IPD126 polypeptides, without altering the biological activity of the proteins. Thus, variant nucleic acid molecules can be created by introducing one or more nucleotide substitutions, additions and/or deletions into the corresponding nucleic acid sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleic acid sequences are also encompassed by the present disclosure.

The polynucleotides of the disclosure and fragments thereof are optionally used as substrates for a variety of recombination and recursive recombination reactions, in addition to standard cloning methods as set forth in, e.g., Ausubel, Berger and Sambrook, i.e., to produce additional pesticidal polypeptide homologues and fragments thereof with desired properties. A variety of such reactions are known. Methods for producing a variant of any nucleic acid listed herein comprising recursively recombining such polynucleotide with a second (or more) polynucleotide, thus forming a library of variant polynucleotides are also embodiments of the disclosure, as are the libraries produced, the cells comprising the libraries and any recombinant polynucleotide produced by such methods. Additionally, such methods optionally comprise selecting a variant polynucleotide from such libraries based on pesticidal activity, as is wherein such recursive recombination is done in vitro or in vivo.

The nucleotide sequences of the embodiments can also be used to isolate corresponding sequences from a bacterial source. In this manner, methods such as PCR, hybridization, and the like can be used to identify such sequences based on their sequence homology to the sequences set forth herein. Sequences that are selected based on their sequence identity to the entire sequences set forth herein or to fragments thereof are encompassed by the embodiments. Such sequences include sequences that are orthologs of the disclosed sequences. The term “orthologs” refers to genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species.

To identify potential IPD126 polypeptides from bacterium collections, the bacterial cell lysates can be screened with antibodies generated against IPD126 using Western blotting and/or ELISA methods. This type of assay can be performed in a high throughput fashion. Positive samples can be further analyzed by various techniques such as antibody based protein purification and identification. Methods of generating antibodies are well known in the art as discussed infra.

In hybridization methods, all or part of the pesticidal nucleic acid sequence can be used to screen cDNA or genomic libraries. Methods for construction of such cDNA and genomic libraries are generally known in the art and are disclosed in Sambrook and Russell, (2001), supra. The so-called hybridization probes may be genomic DNA fragments, cDNA fragments, RNA fragments or other oligonucleotides and may be labeled with a detectable group such as 32P or any other detectable marker, such as other radioisotopes, a fluorescent compound, an enzyme or an enzyme co-factor. Probes for hybridization can be made by labeling synthetic oligonucleotides based on the IPD126 polypeptide-encoding nucleic acid sequences disclosed herein. Degenerate primers designed on the basis of conserved nucleotides or amino acid residues in the nucleic acid sequence or encoded amino acid sequence can additionally be used. The probe typically comprises a region of nucleic acid sequence that hybridizes under stringent conditions to at least about 12, at least about 25, at least about 50, 75, 100, 125, 150, 175 or 200 consecutive nucleotides of nucleic acid sequences encoding IPD126 polypeptides of the disclosure or a fragment or variant thereof. Methods for the preparation of probes for hybridization and stringency conditions are generally known in the art and are disclosed in Sambrook and Russell, (2001), supra, herein incorporated by reference.

### Antibodies

Antibodies to an IPD126 polypeptide of the embodiments or to variants or fragments thereof are also encompassed. The antibodies of the disclosure include polyclonal and monoclonal antibodies as well as fragments thereof which retain their ability to bind to an IPD126 polypeptide. An antibody, monoclonal antibody or fragment thereof is said to be capable of binding a molecule if it is capable of specifically reacting with the molecule to thereby bind the molecule to the antibody, monoclonal antibody or fragment thereof. The term “antibody” (Ab) or “monoclonal antibody” (Mab) is meant to include intact molecules as well as fragments or binding regions or domains thereof (such as, for example, Fab and F(ab).sub.2 fragments) which are capable of binding hapten. Such fragments are typically produced by proteolytic cleavage, such as papain or pepsin. Alternatively, hapten-binding fragments can be produced through the application of recombinant DNA technology or through synthetic chemistry. Methods for the preparation of the antibodies of the present disclosure are generally known in the art. For example, see, Antibodies, A Laboratory Manual, Ed Harlow and David Lane (eds.) Cold Spring Harbor Laboratory, N.Y. (1988), as well as the references cited therein. Standard reference works setting forth the general principles of immunology include: Klein, J. Immunology: The Science of Cell-Noncell Discrimination, John Wiley & Sons, N.Y. (1982); Dennett, et al., Monoclonal Antibodies, Hybridoma: A New Dimension in Biological Analyses, Plenum Press, N.Y. (1980) and Campbell, “Monoclonal Antibody Technology,” In Laboratory Techniques in Biochemistry and Molecular Biology, Vol. 13, Burdon, et al., (eds.), Elsevier, Amsterdam (1984). See also, U.S. Pat. Nos. 4,196,265; 4,609,893; 4,713,325; 4,714,681; 4,716,111; 4,716,117 and 4,720,459. Antibodies against IPD126 polypeptides or antigen-binding portions thereof can be produced by a variety of techniques, including conventional monoclonal antibody methodology, for example the standard somatic cell hybridization technique of Kohler and Milstein, (1975) Nature 256:495. Other techniques for producing monoclonal antibody can also be employed such as viral or oncogenic transformation of B lymphocytes. An animal system for preparing hybridomas is a murine system. Immunization protocols and techniques for isolation of immunized splenocytes for fusion are known in the art. Fusion partners (e.g., murine myeloma cells) and fusion procedures are also known. The antibody and monoclonal antibodies of the disclosure can be prepared by utilizing an IPD126 polypeptide as antigens.

A kit for detecting the presence of an IPD126 polypeptide or detecting the presence of a nucleotide sequence encoding an IPD126 polypeptide in a sample is provided. In one embodiment, the kit provides antibody-based reagents for detecting the presence of an IPD126 polypeptide in a tissue sample. In another embodiment, the kit provides labeled nucleic acid probes useful for detecting the presence of one or more polynucleotides encoding an IPD126 polypeptide. The kit is provided along with appropriate reagents and controls for carrying out a detection method, as well as instructions for use of the kit.

### Receptor Identification and Isolation

Receptors to the IPD126 polypeptides of the embodiments or to variants or fragments thereof are also encompassed. Methods for identifying receptors are known in the art (see, Hofmann, et. al., (1988) Eur. J. Biochem. 173:85-91; Gill, et al., (1995) J. Biol. Chem. 27277-27282) and can be employed to identify and isolate the receptor that recognizes the IPD126 polypeptide using the brush-border membrane vesicles from susceptible insects. In addition to the radioactive labeling method listed in the cited literatures, an IPD126 polypeptide can be labeled with fluorescent dye and other common labels such as streptavidin. Brush-border membrane vesicles (BBMV) of susceptible insects such as soybean looper and stink bugs can be prepared according to the protocols listed in the references of Hofmann and Gill above and separated on SDS-PAGE gel and blotted on suitable membrane. Labeled IPD126 polypeptide can be incubated with blotted membrane of BBMV and labeled IPD126 polypeptide can be identified with the labeled reporters. Identification of protein band(s) that interact with the IPD126 polypeptide can be detected by N-terminal amino acid gas phase sequencing or mass spectrometry based protein identification method (Patterson, (1998) 10.22, 1-24, Current Protocol in Molecular Biology published by John Wiley & Son Inc). Once the protein is identified, the corresponding gene can be cloned from genomic DNA or cDNA library of the susceptible insects and binding affinity can be measured directly with the IPD126 polypeptide. Receptor function for insecticidal activity by the IPD126 polypeptide can be verified by RNAi type of gene knock out method (Rajagopal, et al., (2002) J. Biol. Chem. 277:46849-46851).

### Nucleotide Constructs, Expression Cassettes and Vectors

The use of the term “nucleotide constructs” herein is not intended to limit the embodiments to nucleotide constructs comprising DNA. Those of ordinary skill in the art will recognize that nucleotide constructs, particularly polynucleotides and oligonucleotides composed of ribonucleotides and combinations of ribonucleotides and deoxyribonucleotides, may also be employed in the methods disclosed herein. The nucleotide constructs, nucleic acids, and nucleotide sequences of the embodiments additionally encompass all complementary forms of such constructs, molecules, and sequences. Further, the nucleotide constructs, nucleotide molecules, and nucleotide sequences of the embodiments encompass all nucleotide constructs, molecules, and sequences which can be employed in the methods of the embodiments for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs, nucleic acids, and nucleotide sequences of the embodiments also encompass all forms of nucleotide constructs including, but not limited to, single-stranded forms, double-stranded forms, hairpins, stem-and-loop structures and the like.

A further embodiment relates to a transformed organism such as an organism selected from plant and insect cells, bacteria, yeast, baculovirus, protozoa, nematodes and algae. The transformed organism comprises a DNA molecule of the embodiments, an expression cassette comprising the DNA molecule or a vector comprising the expression cassette, which may be stably incorporated into the genome of the transformed organism.

The sequences of the embodiments are provided in DNA constructs for expression in the organism of interest. The construct will include 5′ and 3′ regulatory sequences operably linked to a sequence of the embodiments. The term “operably linked” as used herein refers to a functional linkage between a promoter and a second sequence, wherein the promoter sequence initiates and mediates transcription of the DNA sequence corresponding to the second sequence. Generally, operably linked means that the nucleic acid sequences being linked are contiguous and where necessary to join two protein coding regions in the same reading frame. The construct may additionally contain at least one additional gene to be cotransformed into the organism. Alternatively, the additional gene(s) can be provided on multiple DNA constructs.

Such a DNA construct is provided with a plurality of restriction sites for insertion of the IPD126 polypeptide gene sequence of the disclosure to be under the transcriptional regulation of the regulatory regions. The DNA construct may additionally contain selectable marker genes.

The DNA construct will generally include in the 5′ to 3′ direction of transcription: a transcriptional and translational initiation region (i.e., a promoter), a DNA sequence of the embodiments, and a transcriptional and translational termination region (i.e., termination region) functional in the organism serving as a host. The transcriptional initiation region (i.e., the promoter) may be native, analogous, foreign or heterologous to the host organism and/or to the sequence of the embodiments. Additionally, the promoter may be the natural sequence or alternatively a synthetic sequence. The term “foreign” as used herein indicates that the promoter is not found in the native organism into which the promoter is introduced. Where the promoter or any other nucleotide or amino acid sequence is “foreign” or “heterologous” to the sequence of the embodiments, it is intended that the nucleotide or amino acid sequence is not the native or naturally occurring promoter or nucleotide sequence for the operably linked sequence of the embodiments. As used herein, a chimeric gene comprises a coding sequence operably linked to a transcription initiation region that is heterologous to the coding sequence. Where the promoter is a native or natural sequence, the expression of the operably linked sequence is altered from the wild-type expression, which results in an alteration in phenotype.

In some embodiments the DNA construct comprises a polynucleotide encoding an IPD126 polypeptide of the embodiments. In some embodiments the DNA construct comprises a polynucleotide encoding a fusion protein comprising an IPD126 polypeptide of the embodiments.

In some embodiments the DNA construct may also include a transcriptional enhancer sequence. As used herein, the term an “enhancer” refers to a DNA sequence which can stimulate promoter activity, and may be an innate element of the promoter or a heterologous element inserted to enhance the level or tissue-specificity of a promoter. Various enhancers are known in the art including for example, introns with gene expression enhancing properties in plants (US Patent Application Publication Number 2009/0144863, the ubiquitin intron (i.e., the maize ubiquitin intron 1 (see, for example, NCBI sequence S94464)), the omega enhancer or the omega prime enhancer (Gallie, et al., (1989)Molecular Biology of RNA ed. Cech (Liss, New York) 237-256 and Gallie, et al., (1987) Gene 60:217-25), the CaMV 35S enhancer (see, e.g., Benfey, et al., (1990) EMBO J. 9:1685-96) and the enhancers of U.S. Pat. No. 7,803,992 may also be used. The above list of transcriptional enhancers is not meant to be limiting. Any appropriate transcriptional enhancer can be used in the embodiments.

The termination region may be native with the transcriptional initiation region, may be native with the operably linked DNA sequence of interest, may be native with the plant host or may be derived from another source (i.e., foreign or heterologous to the promoter, the sequence of interest, the plant host or any combination thereof).

Convenient termination regions are available from the Ti-plasmid of A. tumefaciens, such as the octopine synthase and nopaline synthase termination regions. See also, Guerineau, et al., (1991)Mol. Gen. Genet. 262:141-144; Proudfoot, (1991) Cell 64:671-674; Sanfacon, et al., (1991) Genes Dev. 5:141-149; Mogen, et al., (1990)Plant Cell 2:1261-1272; Munroe, et al., (1990) Gene 91:151-158; Ballas, et al., (1989)Nucleic Acids Res. 17:7891-7903 and Joshi, et al., (1987)Nucleic Acid Res. 15:9627-9639.

Where appropriate, a nucleic acid may be optimized for increased expression in the host organism. Thus, where the host organism is a plant, the synthetic nucleic acids can be synthesized using plant-preferred codons for improved expression. See, for example, Campbell and Gowri, (1990) Plant Physiol. 92:1-11 for a discussion of host-preferred usage. For example, although nucleic acid sequences of the embodiments may be expressed in both monocotyledonous and dicotyledonous plant species, sequences can be modified to account for the specific preferences and GC content preferences of monocotyledons or dicotyledons as these preferences have been shown to differ (Murray et al. (1989) Nucleic Acids Res. 17:477-498). Thus, the maize-preferred for a particular amino acid may be derived from known gene sequences from maize. Maize usage for 28 genes from maize plants is listed in Table 4 of Murray, et al., supra. Methods are available in the art for synthesizing plant-preferred genes. See, for example, Murray, et al., (1989) Nucleic Acids Res. 17:477-498, and Liu H et al. Mol Bio Rep 37:677-684, 2010, herein incorporated by reference. A Zea maize usage table can be also found at kazusa.or.jp//cgi-bin/show.cgi?species=4577, which can be accessed using the www prefix. A Glycine max usage table can be found at kazusa.or.jp//cgi-bin/show.cgi?species=3847&aa=1&style=N, which can be accessed using the www prefix.

In some embodiments the recombinant nucleic acid molecule encoding an IPD126 polypeptide has maize optimized codons.

Additional sequence modifications are known to enhance gene expression in a cellular host. These include elimination of sequences encoding spurious polyadenylation signals, exon-intron splice site signals, transposon-like repeats, and other well-characterized sequences that may be deleterious to gene expression. The GC content of the sequence may be adjusted to levels average for a given cellular host, as calculated by reference to known genes expressed in the host cell. The term “host cell” as used herein refers to a cell which contains a vector and supports the replication and/or expression of the expression vector is intended. Host cells may be prokaryotic cells such as E coli or eukaryotic cells such as yeast, insect, amphibian or mammalian cells or monocotyledonous or dicotyledonous plant cells. An example of a monocotyledonous host cell is a maize host cell. When possible, the sequence is modified to avoid predicted hairpin secondary mRNA structures.

In preparing the expression cassette, the various DNA fragments may be manipulated so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, e.g., transitions and transversions, may be involved.

A number of promoters can be used in the practice of the embodiments. The promoters can be selected based on the desired outcome. The nucleic acids can be combined with constitutive, tissue-preferred, inducible or other promoters for expression in the host organism. Suitable constitutive promoters for use in a plant host cell include, for example, the core promoter of the Rsyn7 promoter and other constitutive promoters disclosed in WO 1999/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EAMBO J. 3:2723-2730); ALS promoter (U.S. Pat. No. 5,659,026) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142 and 6,177,611.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones and 2,4-dichlorophenoxyacetate (2,4-D). Additional examples of suitable selectable marker genes include, but are not limited to, genes encoding resistance to chloramphenicol (Herrera Estrella, et al., (1983) FMBO J. 2:987-992); methotrexate (Herrera Estrella, et al., (1983) Nature 303:209-213 and Meijer, et al., (1991) Plant Mol. Biol. 16:807-820); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-136); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) FMBO J. 6:2513-2518). See generally, Yarranton, (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad Sci. USA 86:5400-5404; Fuerst, et al., (1989) Proc. Natl. Acad Sci. USA 86:2549-2553; Deuschle, et al., (1990) Science 248:480-483; Gossen, (1993) Ph.D. Thesis, University of Heidelberg; Reines, et al., (1993) Proc. Natl. Acad Sci. USA 90:1917-1921; Labow, et al., (1990) Mol. Cell. Biol. 10:3343-3356; Zambretti, et al., (1992) Proc. Natl. Acad Sci. USA 89:3952-3956; Baim, et al., (1991) Proc. Natl. Acad Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Nall. Acad Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin) and Gill, et al., (1988) Nature 334:721-724.

The above list of selectable marker genes is not meant to be limiting. Any selectable marker gene can be used in the embodiments.

### Plant Transformation

The methods of the embodiments involve introducing a polypeptide or polynucleotide into a plant. “Introducing” as used herein means presenting to the plant the polynucleotide or polypeptide in such a manner that the sequence gains access to the interior of a cell of the plant. The methods of the embodiments do not depend on a particular method for introducing a polynucleotide or polypeptide into a plant, only that the polynucleotide(s) or polypeptide(s) gains access to the interior of at least one cell of the plant. Methods for introducing polynucleotide(s) or polypeptide(s) into plants are known in the art including, but not limited to, stable transformation methods, transient transformation methods, and virus-mediated methods.

“Stable transformation” as used herein means that the nucleotide construct introduced into a plant integrates into the genome of the plant and is capable of being inherited by the progeny thereof. “Transient transformation” as used herein means that a polynucleotide is introduced into the plant and does not integrate into the genome of the plant or a polypeptide is introduced into a plant. “Plant” as used herein refers to whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells and pollen).

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (U.S. Pat. Nos. 5,563,055 and 5,981,840), direct gene transfer (Paszkowski, et al., (1984) EMBO J. 3:2717-2722) and ballistic particle acceleration (see, for example, U.S. Pat. Nos. 4,945,050; 5,879,918; 5,886,244 and 5,932,782; Tomes, et al., (1995) in Plant Cell, Tissue, and Organ Culture: Fundamental Methods, ed. Gamborg and Phillips, (Springer-Verlag, Berlin) and McCabe, et al., (1988) Biotechnology 6:923-926) and Led transformation (WO 00/28058). For potato transformation see, Tu, et al., (1998) Plant Molecular Biology 37:829-838 and Chong, et al., (2000) Transgenic Research 9:71-78. Additional transformation procedures can be found in Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens).

In specific embodiments, the sequences of the embodiments can be provided to a plant using a variety of transient transformation methods. Such transient transformation methods include, but are not limited to, the introduction of the IPD126 polynucleotide or variants and fragments thereof directly into the plant or the introduction of the IPD126 polypeptide transcript into the plant. Such methods include, for example, microinjection or particle bombardment. See, for example, Crossway, et al., (1986) Mol Gen. Genet. 202:179-185; Nomura, et al., (1986) Plant Sci. 44:53-58; Hepler, et al., (1994) Proc. Natl. Acad. Sci. 91:2176-2180 and Hush, et al., (1994) The Journal of Cell Science 107:775-784. Alternatively, the IPD126 polynucleotide can be transiently transformed into the plant using techniques known in the art. Such techniques include viral vector system and the precipitation of the polynucleotide in a manner that precludes subsequent release of the DNA. Thus, transcription from the particle-bound DNA can occur, but the frequency with which it is released to become integrated into the genome is greatly reduced. Such methods include the use of particles coated with polyethylimine (PEI; Sigma #P3143).

Methods are known in the art for the targeted insertion of a polynucleotide at a specific location in the plant genome. In one embodiment, the insertion of the polynucleotide at a desired genomic location is achieved using a site-specific recombination system. See, for example, WO 1999/25821, WO 1999/25854, WO 1999/25840, WO 1999/25855 and WO 1999/25853. Briefly, the polynucleotide of the embodiments can be contained in transfer cassette flanked by two non-identical recombination sites. The transfer cassette is introduced into a plant have stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

Plant transformation vectors may be comprised of one or more DNA vectors needed for achieving plant transformation. For example, it is a common practice in the art to utilize plant transformation vectors that are comprised of more than one contiguous DNA segment. These vectors are often referred to in the art as “binary vectors”. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker gene and the pesticidal gene are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux, (2000) Trends in Plant Science 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethylene glycol, etc.

In general, plant transformation methods involve transferring heterologous DNA into target plant cells (e.g., immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Following integration of heterologous foreign DNA into plant cells, one then applies a maximum threshold level of appropriate selection in the medium to kill the untransformed cells and separate and proliferate the putatively transformed cells that survive from this selection treatment by transferring regularly to a fresh medium. By continuous passage and challenge with appropriate selection, one identifies and proliferates the cells that are transformed with the plasmid vector. Molecular and biochemical methods can then be used to confirm the presence of the integrated heterologous gene of interest into the genome of the transgenic plant.

Explants are typically transferred to a fresh supply of the same medium and cultured routinely. Subsequently, the transformed cells are differentiated into shoots after placing on regeneration medium supplemented with a maximum threshold level of selecting agent. The shoots are then transferred to a selective rooting medium for recovering rooted shoot or plantlet. The transgenic plantlet then grows into a mature plant and produces fertile seeds (e.g., Hiei, et al., (1994) The Plant Journal 6:271-282; Ishida, et al., (1996) Nature Biotechnology 14:745-750). Explants are typically transferred to a fresh supply of the same medium and cultured routinely. A general description of the techniques and methods for generating transgenic plants are found in Ayres and Park, (1994) Critical Reviews in Plant Science 13:219-239 and Bommineni and Jauhar, (1997) Maydica 42:107-120. Since the transformed material contains many cells; both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants.

The cells that have been transformed may be grown into plants in accordance with conventional ways. See, for example, McCormick, et al., (1986) Plant Cell Reports 5:81-84. These plants may then be grown, and either pollinated with the same transformed strain or different strains, and the resulting hybrid having constitutive or inducible expression of the desired phenotypic characteristic identified. Two or more generations may be grown to ensure that expression of the desired phenotypic characteristic is stably maintained and inherited and then seeds harvested to ensure that expression of the desired phenotypic characteristic has been achieved.

The embodiments further relate to plant-propagating material of a transformed plant of the embodiments including, but not limited to, seeds, tubers, corms, bulbs, leaves and cuttings of roots and shoots.

The embodiments may be used for transformation of any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago saliva), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Fleusine coracana)), sunflower (Helianthus annuus), safflower (Carthamus tinctorius), wheat (Triticum aestivum), soybean (Glycine mar), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pucherrima), and chrysanthemum. Conifers that may be employed in practicing the embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Plants of the embodiments include crop plants (for example, corn, alfalfa, sunflower, Brassica, soybean, cotton, safflower, peanut, sorghum, wheat, millet, tobacco, etc.), such as corn and soybean plants.

Turf grasses include, but are not limited to: annual bluegrass (Poa annua); annual ryegrass (Lolium multiflorum); Canada bluegrass (Poa compressa); Chewing's fescue (Festuca rubra); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerata); perennial ryegrass (Lolium perenne); red fescue (Festuca rubra); redtop (Agrostis alba); rough bluegrass (Poa trivialis); sheep fescue (Festuca ovina); smooth bromegrass (Bromus inermis); tall fescue (Festuca arundinacea); timothy (Phleum pratense); velvet bentgrass (Agrostis canina); weeping alkaligrass (Puccinellia distans); western wheatgrass (Agropyron smithii); Bermuda grass (Cynodon spp.); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

Plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive, etc. Leguminous plants include beans and peas. Beans include guar, locust bean, fenugreek, soybean, garden beans, cowpea, mung bean, lima bean, fava bean, lentils, chickpea, etc.

Following introduction of heterologous foreign DNA into plant cells, the transformation or integration of heterologous gene in the plant genome is confirmed by various methods such as analysis of nucleic acids, proteins and metabolites associated with the integrated gene.

PCR analysis is a rapid method to screen transformed cells, tissue or shoots for the presence of incorporated gene at the earlier stage before transplanting into the soil (Sambrook and Russell, (2001) Molecular Cloning: A Laboratory Manual. Cold Spring Harbor Laboratory Press, Cold Spring Harbor, NY). PCR is carried out using oligonucleotide primers specific to the gene of interest or Agrobacterium vector background, etc.

Plant transformation may be confirmed by Southern blot analysis of genomic DNA (Sambrook and Russell, (2001) supra). In Northern blot analysis, RNA is isolated from specific tissues of transformant, fractionated in a formaldehyde agarose gel, and blotted onto a nylon filter according to standard procedures that are routinely used in the art (Sambrook and Russell, (2001) supra). Expression of RNA encoded by the pesticidal gene is then tested by hybridizing the filter to a radioactive probe derived from a pesticidal gene, by methods known in the art (Sambrook and Russell, (2001) supra). Western blot, biochemical assays and the like may be carried out on the transgenic plants to confirm the presence of protein encoded by the pesticidal gene by standard procedures (Sambrook and Russell, 2001, supra) using antibodies that bind to one or more epitopes present on the IPD126 polypeptide.

Methods to Introduce Genome Editing Technologies into Plants

In some embodiments, the disclosed IPD126 polynucleotide compositions can be introduced into the genome of a plant using genome editing technologies, or previously introduced IPD126 polynucleotides in the genome of a plant may be edited using genome editing technologies. For example, the disclosed polynucleotides can be introduced into a desired location in the genome of a plant through the use of double-stranded break technologies such as TALENs, meganucleases, zinc finger nucleases, CRISPR-Cas, and the like. For example, the disclosed polynucleotides can be introduced into a desired location in a genome using a CRISPR-Cas system, for the purpose of site-specific insertion. The desired location in a plant genome can be any desired target site for insertion, such as a genomic region amenable for breeding or may be a target site located in a genomic window with an existing trait of interest. Existing traits of interest could be either an endogenous trait or a previously introduced trait.

In some embodiments, where the disclosed IPD126 polynucleotide has previously been introduced into a genome, genome editing technologies may be used to alter or modify the introduced polynucleotide sequence. Site specific modifications that can be introduced into the disclosed IPD126 polynucleotide compositions include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. Such technologies can be used to modify the previously introduced polynucleotide through the insertion, deletion or substitution of nucleotides within the introduced polynucleotide. Alternatively, double-stranded break technologies can be used to add additional nucleotide sequences to the introduced polynucleotide. Additional sequences that may be added include, additional expression elements, such as enhancer and promoter sequences. In another embodiment, genome editing technologies may be used to position additional insecticidally-active proteins in proximity to the disclosed IPD126 polynucleotide compositions disclosed herein within the genome of a plant, in order to generate molecular stacks of insecticidally-active proteins.

An “altered target site,” “altered target sequence.” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

### Stacking of Traits in Transgenic Plant

Transgenic plants may comprise a stack of one or more insecticidal polynucleotides disclosed herein with one or more additional polynucleotides resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene and co-transformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Co-transformation of genes can be carried out using single transformation vectors comprising multiple genes or genes carried separately on multiple vectors. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO 1999/25821, WO 1999/25854, WO 1999/25840, WO 1999/25855 and WO 1999/25853, all of which are herein incorporated by reference.

In some embodiments, one or more of the polynucleotides encoding the IPD126 polypeptide(s) disclosed herein, alone or stacked with one or more additional insect resistance traits can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like). Thus, the polynucleotide embodiments can be used to provide a complete agronomic package of improved crop quality with the ability to flexibly and cost effectively control any number of agronomic pests.

Transgenes useful for stacking include but are not limited to: transgenes that confer resistance to an herbicide; transgenes that confer or contribute to an altered grain characteristic; genes that control male-sterility; genes that create a site for site specific dna integration; genes that affect abiotic stress resistance; genes that confer increased yield genes that confer plant digestibility; and transgenes that confer resistance to insects or disease.

Examples of transgenes that confer resistance to insects include genes encoding a Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC* Accession Numbers 40098, 67136, 31995 and 31998. Other non-limiting examples of Bacillus thuringiensis transgenes being genetically engineered are given in the following patents and patent applications: U.S. Pat. Nos. 5,188,960; 5,689,052; 5,880,275; 5,986,177; 6,023,013, 6,060,594, 6,063,597, 6,077,824, 6,620,988, 6,642,030, 6,713,259, 6,893,826, 7,105,332; 7,179,965, 7,208,474; 7,227,056, 7,288,643, 7,323,556, 7,329,736, 7,449,552, 7,468,278, 7,510,878, 7,521,235, 7,544,862, 7,605,304, 7,696,412, 7,629,504, 7,705,216, 7,772,465, 7,790,846, 7,858,849 and WO 1991/14778; WO 1999/31248; WO 2001/12731; WO 1999/24581 and WO 1997/40162.

Genes encoding pesticidal proteins may also be stacked including but are not limited to: insecticidal proteins from Pseudomonas sp. such as PSEEN3174 (Monalysin, (2011) PLoS Pathogens, 7:1-13), from Pseudomonas protegens strain CHAO and Pf-5 (previously fluorescens) (Pechy-Tarr, (2008) Environmental Microbiology 10:2368-2386: GenBank Accession No. EU400157); from Pseudomonas taiwanensis (Liu, et al., (2010) J. Agric. Food Chem. 58:12343-12349) and from Pseudomonas pseudoalcaligenes (Zhang, et al., (2009) Annals ofMicrobiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxinology Journal 3:101-118 and Morgan, et al., (2001) Applied and Envir. Micro. 67:2062-2069), U.S. Pat. Nos. 6,048,838, and 6,379,946; a PIP-1 polypeptide of U.S. Pat. No. 9,688,730; an AfIP-1A and/or AfIP-1B polypeptide of U.S. Pat. No. 9,475,847; a PIP-47 polypeptide of US Publication Number US20160186204; an IPD045 polypeptide, an IPD064 polypeptide, an IPD074 polypeptide, an IPD075 polypeptide, and an IPD077 polypeptide of PCT Publication Number WO 2016/114973; an IPD080 polypeptide of PCT Serial Number PCT/US17/56517; an IPD078 polypeptide, an IPD084 polypeptide, an IPD085 polypeptide, an IPD086 polypeptide, an IPD087 polypeptide, an IPD088 polypeptide, and an IPD089 polypeptide of Serial Number PCT/US17/54160; PIP-72 polypeptide of US Patent Publication Number US20160366891; a PtIP-50 polypeptide and a PtIP-65 polypeptide of US Publication Number US20170166921; an IPD098 polypeptide, an IPD059 polypeptide, an IPD108 polypeptide, an IPD109 polypeptide of U.S. Ser. No. 62/521,084; a PtIP-83 polypeptide of US Publication Number US20160347799; a PtIP-96 polypeptide of US Publication Number US20170233440; an IPD079 polypeptide of PCT Publication Number WO2017/23486; an IPD082 polypeptide of PCT Publication Number WO 2017/105987, an IPDO90 polypeptide of Serial Number PCT/US17/30602, an IPD093 polypeptide of U.S. Ser. No. 62/434,020; an IPD103 polypeptide of Serial Number PCT/US17/39376; an IPD101 polypeptide of U.S. Ser. No. 62/438,179; an IPD121 polypeptide of US Serial Number U.S. 62/508,514, and S-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry50, Cry51, Cry52, Cry53, Cry 54, Cry55, Cry56, Cry57, Cry58, Cry59, Cry60, Cry61, Cry62, Cry63, Cry64, Cry65, Cry66, Cry67, Cry68, Cry69, Cry70, Cry71, and Cry 72 classes of S-endotoxin genes and the B. thuringiensis cytolytic Cyt1 and Cyt2 genes.

Examples of δ-endotoxins also include but are not limited to Cry1A proteins of U.S. Pat. Nos. 5,880,275 and 7,858,849; a DIG-3 or DIG-11 toxin (N-terminal deletion of a-helix 1 and/or a-helix 2 variants of Cry proteins such as Cry1A) of US Patent Numbers 8,304,604 and 8.304,605, CrylB of U.S. patent application Ser. No. 10/525,318; CrylC of U.S. Pat. No. 6,033,874; Cry1F of U.S. Pat. Nos. 5,188,960, 6,218,188; Cry1A/F chimeras of U.S. Pat. Nos. 7,070,982; 6,962,705 and 6,713,063); a Cry2 protein such as Cry2Ab protein of U.S. Pat. No. 7,064,249); a Cry3A protein including but not limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as such as members of the Cry9A, Cry9B, Cry9C, Cry9D, Cry9E, and Cry9F families; a Cry15 protein of Naimov, et al., (2008) Applied and Environmental Microbiology 74:7145-7151; a Cry22, a Cry34Ab1 protein of U.S. Pat. Nos. 6,127,180, 6,624,145 and 6,340,593; a CryET33 and CryET34 protein of U.S. Pat. Nos. 6,248,535, 6,326,351, 6,399,330, 6,949,626, 7,385,107 and 7,504,229; a CryET33 and CryET34 homologs of US Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein, a Cry 51 protein, a Cry binary toxin; a TIC901 or related toxin; TIC807 of US 2008/0295207; ET29, ET37, TIC809, TIC810, TIC812, TIC127, TIC128 of PCT US 2006/033867; AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMII-020, and AXMI-021 of WO 2006/083891; AXMI-010 of WO 2005/038032; AXNI-003 of WO 2005/021585; AXMI-008 of US 2004/0250311; AXMII-006 of US 2004/0216186; AXMI-007 of US 2004/0210965; AXMI-009 of US 2004/0210964; AXMI-014 of US 2004/0197917; AXMI-004 of US 2004/0197916; AXMI-028 and AXMI-029 of WO 2006/119457; AXMI-007, AXMI-008, AXMI-0080rf2, AXMI-009, AXMI-014 and AXMI-004 of WO 2004/074462; AXMI-150 of U.S. Pat. No. 8,084,416; AXMI-205 of US20110023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063, and AXMI-064 of US 2011/0263488; AXMI-R1 and related proteins of US 2010/0197592; AXMI221Z, AXMI222z, AXMII223z, AXMI224z and AXMII225z of WO 2011/103248; AXNM218, AXMI219, AXNM220, AXNM226, AXNM227, AXNM228, AXNM229, AXNI230, and AXNM231 of WO11/103247; AXMI-115, AXMI-113, AXMI-005, AXMI-163 and AXMI-184 of U.S. Pat. No. 8,334,431; AXMI-001, AXMI-002, AXMI-030, AXMI-035, and AXMI-045 of US 2010/0298211; AXMI-066 and AXMI-076 of US2009/0144852; AXMHI128, AXMI130, AXMHI131, AXMHI133, AXMI140, AXMHI141, AXMI142, AXMHI143, AXNII144, AXNII146, AXNII148, AXNII149, AXNII152, AXNII153, AXNII154, AXNII155, AXMHI156, AXMI157, AXMHI158, AXMI162, AXMHI165, AXMHI166, AXMHI167, AXMI168, AXMHI169, AXMI170, AXMHI171, AXMI172, AXNHI173, AXMI174, AXMHI175, AXMI176, AXNII177, AXNII178, AXNII179, AXNII180, AXNII181, AXNII182, AXNII185, AXMI186, AXNMI187, AXMI188, AXMHI189 of U.S. Pat. No. 8,318,900; AXMI079, AXNI080, AXMI081, AXMI082, AXMI091, AXMI092, AXMI096, AXMI097, AXMI098, AXMI099, AXNII100, AXMII101, AXNII102, AXMII103, AXMII104, AXMII107, AXMII108, AXNII109, AXMI110, AXMI111, AXHI112, AXMI114, AXMI116, AXMI117, AXMI118, AXMI119, AXNI120, AXMI121, AXMI122, AXMI123, AXMI124, AXNI1257, AXMI1268, AXMI127, AXNII129, AXNII164, AXMI151, AXMI161, AXNII183, AXNII132, AXMI138, AXMI137 of US 2010/0005543; and Cry proteins such as CrylA and Cry3A having modified proteolytic sites of U.S. Pat. No. 8,319,019; and a CrylAc, Cry2Aa and Cry1Ca toxin protein from Bacillus thuringiensis strain VBTS 2528 of US Patent Application Publication Number 2011/0064710. Other Cry proteins are well known to one skilled in the art (see, Crickmore, et al., “Bacillus thuringiensis toxin nomenclature” (2011), at lifesci.sussex.ac.uk/home/Neil_Crickmore/Bt/which can be accessed on the world-wide web using the “www” prefix). The insecticidal activity of Cry proteins is well known to one skilled in the art (for review, see, van Frannkenhuyzen, (2009) J. Invert. Path. 101:1-16). The use of Cry proteins as transgenic plant traits is well known to one skilled in the art and Cry-transgenic plants including but not limited to Cry1Ac, Cry1Ac+Cry2Ab, Cry1Ab, Cry1A.105, Cry1F, CrylFa2, Cry1F+Cry1Ac, Cry2Ab, Cry3A, mCry3A, Cry3Bb1, Cry34Ab1, Cry35Ab1, Vip3A, mCry3A, Cry9c and CBI-Bt have received regulatory approval (see, Sanahuja, (2011) Plant Biotech Journal 9:283-300 and the CERA (2010) GM Crop Database Center for Environmental Risk Assessment (CERA), ILSI Research Foundation, Washington D.C. at cera-gmc.org/rndex.php?action=gm_crop_database which can be accessed on the world-wide web using the “www” prefix). More than one pesticidal proteins well known to one skilled in the art can also be expressed in plants such as Vip3Ab & Cry1Fa (US2012/0317682), Cry1BE & Cry1F (US2012/0311746), Cry1CA & Cry1AB (US2012/0311745), Cry1F & CryCa (US2012/0317681), Cry1DA & Cry1BE (US2012/0331590), Cry1DA & Cry1Fa (US2012/0331589), Cry1AB & Cry1BE (US2012/0324606), and Cry1Fa & Cry2Aa, Cry1I or Cry1E (US2012/0324605). Pesticidal proteins also include insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413). Pesticidal proteins also include VIP (vegetative insecticidal proteins) toxins of U.S. Pat. Nos. 5,877,012, 6,107,279, 6,137,033, 7,244,820, 7,615,686, and 8,237,020, and the like. Other VIP proteins are well known to one skilled in the art (see, lifesci.sussex.ac.uk/home/Neil_Crickmore/Bt/vip.html which can be accessed on the world-wide web using the “www” prefix). Pesticidal proteins also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptBlXb and XptClWi. Examples of Class C proteins are TccC, XptC1Xb and XptB1Wi. Pesticidal proteins also include spider, snake and scorpion venom proteins. Examples of spider venom peptides include but are not limited to lycotoxin-1 peptides and mutants thereof (U.S. Pat. No. 8,334,366).

Further transgenes that confer resistance to insects may down-regulation of expression of target genes in insect pest species by interfering ribonucleic acid (RNA) molecules through RNA interference. RNA interference refers to the process of sequence-specific post-transcriptional gene silencing in animals mediated by short interfering RNAs (siRNAs) (Fire, et al., (1998) Nature 391:806). RNAi transgenes may include but are not limited to expression of dsRNA, siRNA, miRNA, iRNA, antisense RNA, or sense RNA molecules that down-regulate expression of target genes in insect pests. PCT Publication WO 2007/074405 describes methods of inhibiting expression of target genes in invertebrate pests including Colorado potato beetle. PCT Publication WO 2005/110068 describes methods of inhibiting expression of target genes in invertebrate pests including in particular Western corn rootworm as a means to control insect infestation. Furthermore, PCT Publication WO 2009/091864 describes compositions and methods for the suppression of target genes from insect pest species including pests from the Lygus genus. RNAi transgenes are provided for targeting the vacuolar ATPase H subunit, useful for controlling a coleopteran pest population and infestation as described in US Patent Application Publication 2012/0198586. PCT Publication WO 2012/055982 describes ribonucleic acid (RNA or double stranded RNA) that inhibits or down regulates the expression of a target gene that encodes: an insect ribosomal protein such as the ribosomal protein L19, the ribosomal protein L40 or the ribosomal protein S27A; an insect proteasome subunit such as the Rpn6 protein, the Pros 25, the Rpn2 protein, the proteasome beta 1 subunit protein or the Pros beta 2 protein; an insect β-coatomer of the COPI vesicle, the γ-coatomer of the COPI vesicle, the β′—coatomer protein or the ζ-coatomer of the COPI vesicle; an insect Tetraspanine 2 A protein which is a putative transmembrane domain protein; an insect protein belonging to the actin family such as Actin 5C; an insect ubiquitin-5E protein; an insect Sec23 protein which is a GTPase activator involved in intracellular protein transport; an insect crinkled protein which is an unconventional myosin which is involved in motor activity; an insect crooked neck protein which is involved in the regulation of nuclear alternative mRNA splicing; an insect vacuolar H+-ATPase G-subunit protein and an insect Tbp-1 such as Tat-binding protein. PCT publication WO 2007/035650 describes ribonucleic acid (RNA or double stranded RNA) that inhibits or down regulates the expression of a target gene that encodes Snf7. US Patent Application publication 2011/0054007 describes polynucleotide silencing elements targeting RPS10. PCT publication WO 2016/205445 describes polynucleotide silencing elements that reduce fecundity, with target polynucleotides, including NCLB, MAEL, BOULE, and VgR. US Patent Application publication 2014/0275208 and US2015/0257389 describes polynucleotide silencing elements targeting RyanR and PAT3. PCT publications WO/2016/138106, WO 2016/060911, WO 2016/060912, WO 2016/060913, and WO 2016/060914 describe polynucleotide silencing elements targeting COPI coatomer subunit nucleic acid molecules that confer resistance to Coleopteran and Hemipteran pests. US Patent Application Publications 2012/029750, US 20120297501, and 2012/0322660 describe interfering ribonucleic acids (RNA or double stranded RNA) that functions upon uptake by an insect pest species to down-regulate expression of a target gene in said insect pest, wherein the RNA comprises at least one silencing element wherein the silencing element is a region of double-stranded RNA comprising annealed complementary strands, one strand of which comprises or consists of a sequence of nucleotides which is at least partially complementary to a target nucleotide sequence within the target gene. US Patent Application Publication 2012/0164205 describe potential targets for interfering double stranded ribonucleic acids for inhibiting invertebrate pests including: a Chd3 Homologous Sequence, a Beta-Tubulin Homologous Sequence, a 40 kDa V-ATPase Homologous Sequence, a EF1α Homologous Sequence, a 26S Proteosome Subunit p28 Homologous Sequence, a Juvenile Hormone Epoxide Hydrolase Homologous Sequence, a Swelling Dependent Chloride Channel Protein Homologous Sequence, a Glucose-6-Phosphate 1-Dehydrogenase Protein Homologous Sequence, an Act42A Protein Homologous Sequence, a ADP-Ribosylation Factor 1 Homologous Sequence, a Transcription Factor IIB Protein Homologous Sequence, a Chitinase Homologous Sequences, a Ubiquitin Conjugating Enzyme Homologous Sequence, a Glyceraldehyde-3-Phosphate Dehydrogenase Homologous Sequence, an Ubiquitin B Homologous Sequence, a Juvenile Hormone Esterase Homolog, and an Alpha Tubuliln Homologous Sequence.

### Methods for Killing an Insect Pest and Controlling an Insect Population

In some embodiments methods are provided for killing an insect pest, comprising contacting the insect pest, either simultaneously or sequentially, with an insecticidally-effective amount of a recombinant IPD126 polypeptide of the disclosure. In some embodiments methods are provided for killing an insect pest, comprising contacting the insect pest with an insecticidally-effective amount of one or more of a recombinant pesticidal protein of SEQ ID NOS: 19-36, or a variant or insecticidally active fragment thereof.

In some embodiments methods are provided for controlling an insect pest population, comprising contacting the insect pest population, either simultaneously or sequentially, with an insecticidally-effective amount of one or more of a recombinant IPD126 polypeptide of the disclosure. In some embodiments, methods are provided for controlling an insect pest population, comprising contacting the insect pest population with an insecticidally-effective amount of one or more of a recombinant IPD126 polypeptide of SEQ ID NOS: 19-36, or a variant or insecticidally active fragment thereof. As used herein, “controlling a pest population” or “controls a pest” refers to any effect on a pest that results in limiting the damage that the pest causes. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack or deterring the pests from eating the plant.

In some embodiments methods are provided for controlling an insect pest population resistant to a pesticidal protein, comprising contacting the insect pest population, either simultaneously or sequentially, with an insecticidally-effective amount of one or more of a recombinant IPD126 polypeptide of the disclosure. In some embodiments, methods are provided for controlling an insect pest population resistant to a pesticidal protein, comprising contacting the insect pest population with an insecticidally-effective amount of one or more of a recombinant IPD126 polypeptide of SEQ ID NOS: 19-36, or a variant or insecticidally active fragment thereof.

In some embodiments methods are provided for protecting a plant from an insect pest, comprising expressing in the plant or cell thereof at least one recombinant polynucleotide encoding a IPD126 polypeptide of the disclosure. In some embodiments methods are provided for protecting a plant from an insect pest, comprising expressing in the plant or cell thereof a recombinant polynucleotide encoding one or more IPD126 polypeptides of SEQ ID NOS: 19-36, or variants or insecticidally active fragments thereof.

### Insect Resistance Management (IRM) Strategies

Expression of B. thuringiensis-endotoxins in transgenic corn plants has proven to be an effective means of controlling agriculturally important insect pests (Perlak, et al., 1990; 1993). However, in certain instances insects have evolved that are resistant to B. thuringiensis & endotoxins expressed in transgenic plants. Such resistance, should it become widespread, would clearly limit the commercial value of germplasm containing genes encoding such B. thuringiensis δ-endotoxins.

One way of increasing the effectiveness of the transgenic insecticides against target pests and contemporaneously reducing the development of insecticide-resistant pests is to use non-transgenic (i.e., non-insecticidal protein) refuges (a section of non-insecticidal crops/corn) with transgenic crops producing a single insecticidal protein active against target pests. The United States Environmental Protection Agency (epa.gov/oppbppdl/biopesticides/pips/bt_corn_refuge_2006.htm, which can be accessed using the www prefix) publishes the requirements for use with transgenic crops producing a single Bt protein active against target pests. In addition, the National Corn Growers Association, on their website: (ncga.comrnsect-resistance-management-fact-sheet-bt-corn, which can be accessed using the www prefix) also provides similar guidance regarding refuge requirements. Due to losses to insects within the refuge area, larger refuges may reduce overall yield.

Another way of increasing the effectiveness of the transgenic insecticides against target pests and contemporaneously reducing the development of insecticide-resistant pests would be to have a repository of insecticidal genes that are effective against groups of insect pests and which manifest their effects through different modes of action.

Expression in a plant of two or more insecticidal compositions toxic to the same insect species, each insecticide being expressed at efficacious levels would be another way to achieve control of the development of resistance. This is based on the principle that evolution of resistance against two separate modes of action is far more unlikely than only one. Roush, for example, outlines two-toxin strategies, also called “pyramiding” or “stacking,” for management of insecticidal transgenic crops. (The Royal Society. Phil. Trans. R. Soc. Lond. B. (1998) 353:1777-1786). Stacking or pyramiding of two different proteins each effective against the target pests and with little or no cross-resistance can allow for use of a smaller refuge. The US Environmental Protection Agency requires significantly less (generally 5%) structured refuge of non-Bt corn be planted than for single trait products (generally 20%). There are various ways of providing the IRM effects of a refuge, including various geometric planting patterns in the fields and in-bag seed mixtures, as discussed further by Roush.

In some embodiments the IPD126 polypeptides of the disclosure are useful as an insect resistance management strategy in combination (i.e., pyramided) with other pesticidal proteins or other transgenes (i.e., an RNAi trait) including but not limited to Bt toxins, Xenorhabdus sp. or Photorhabdus sp. insecticidal proteins, other insecticidally active proteins, and the like.

Provided are methods of controlling Lepidoptera and/or Coleoptera insect infestation(s) in a transgenic plant that promote insect resistance management, comprising expressing in the plant at least two different insecticidal proteins having different modes of action.

In some embodiments the methods of controlling Lepidoptera and/or Coleoptera insect infestation in a transgenic plant and promoting insect resistance management comprises the presentation of at least one of the IPD126 polypeptide insecticidal proteins to insects in the order Lepidoptera and/or Coleoptera.

In some embodiments the methods of controlling Lepidoptera and/or Coleoptera insect infestation in a transgenic plant and promoting insect resistance management comprises the presentation of at least one of the IPD126 polypeptides of SEQ ID NOS: 19-36, or variants or insecticidally active fragments thereof, insecticidal to insects in the order Lepidoptera and/or Coleoptera.

Also provided are methods of reducing likelihood of emergence of Lepidoptera and/or Coleoptera insect resistance to transgenic plants expressing in the plants insecticidal proteins to control the insect species, comprising expression of at least one of the IPD126 polypeptides to the insect species in combination with a second insecticidal protein to the insect species having different modes of action.

The following examples are offered by way of illustration and not by way of limitation.

### EXAMPLES

### Example 1. Coleoptera Diet-Based Feeding Assays

Western corn rootworm (WCRW, Diabrotica virgifera virgifera) bioassays were conducted using the cell lysates 10 microliter samples mixed with molten low-melt diet (Southland Products Inc., Lake Village, Arkansas) in a 96 well format. WCRW neonates were placed into each well of a 96 well plate. The assay was run four days at 25° C. and then was scored for insect mortality and stunting of insect growth. The scores were noted as dead, severely stunted (little or no growth but alive), stunted (growth to second instar but not equivalent to controls) or no activity.

### Example 2. Strain Isolation, Cultivation and Activity Bioassay

Plant samples were collected from multiple locations in Iowa. Plant samples were broken into smaller pieces and submerged in PBS buffer. After 15 min at low rpm shaking, 100ul of the wash was then serial diluted out and plated on several different isolation agar media. Various bacterial strains were picked and cultured in liquid Trypticase soy medium (Tryptone 17 g/L, enzymatic digest of soy meal 3 g/L, Dextrose 2.5 g/L, Sodium Chloride 5 g/L, K2HPO4 2.5 g/L) overnight at 26° C. with shaking at 250 rpm. The total protein was extracted from cell mass and used for insect bioassay. Some strains, including Pantoea agglomerans strain PMC3671E3-1 (NRRL Deposit No. B-67697), Pantoea agglomerans strain PMC3671E9-1 (NRRL Deposit No. B-67698), and Pantoea agglomerans strain PMCJ4082D4-1 (NRRL Deposit No. B-67699) showed strong insecticidal activity against WCRW. The insecticidal activity was further confirmed with new cultures.

### Example 3. Strain Sequencing and Species Identification

Genomic DNA from strain PMC3671E9-1, PMC3671E3-1, and PMCJ4082D4-1 was extracted with a Sigma Bacterial Genomic DNA Extraction Kit (Cat #NA2110-KT, Sigma-Aldrich, PO Box 14508, St. Louis, MO 63178) according to the manufactures' instructions. The DNA concentration was determined using a NanoDrop Spectrophotometer (Thermo Scientific, 3411 Silverside Road, Bancroft Building, Suite 100, Wilmington, DE 19810) and the genomic DNA was diluted to 50ng/ul with sterile water. The genomes were sequenced with Illumina and PacBio sequencers. The sequences were assembled and annotated. The 16S rDNA (SEQ ID NO: 37, SEQ ID NO: 38, and SEQ ID NO: 39) was extracted from the genome sequence and blasted against NCBI database. The results indicated that PMC3671E9-1, PMC3671E3-1, and and PMCJ4082D4-1 are Pantoea agglomerans strains.

### Example 4. Active Protein Identification and Confirmation

This insecticidal activity of these strains exhibited heat and proteinase sensitivity indicating proteinaceous nature. Insecticidal activity against WCRW was observed from both cell culture supernatant and clear cell lysate from Pantoea agglomerans strain PMC3671E9-1 grown in Terrific Broth and cultured for 2 days at 26° C. with shaking at 250 rpm. 80 ml of Cell supernatant of PMC3671E9-1 was subjected to Tangential Flow Filtration in a 100 kDa MWCO Polyethersulfone (PES) membrane from Spectrum Labs equilibrated in 20 mM Tris-HCl buffer, pH 8.0, 150 mM NaCl (buffer A). The cell supernatant was concentrated, and buffer exchanged into buffer A and a volume of 8 ml was recovered. This material was loaded onto a Superdex 200 26/600 pg column (size exclusion, GE Healthcare) equilibrated in buffer A. Fractions corresponding with insecticidal activity were pooled and buffer exchanged into 1M Ammonium Sulfate, 20 mM Tris-HCl, pH 8 (buffer B) and applied to a Source 15 Phenyl 4.6/100 column (hydrophobic interaction, GE Healthcare) equilibrated in buffer B. Protein was eluted with a linear gradient from 1 M to 0 M ammonium sulfate. Fractions were desalted and subjected for identification of insecticidal activity. SDS-PAGE analysis of fractions with WCRW activity showed a band corresponding with insecticidal activity after staining with SYPRO ruby gel stain (Invitrogen). Mass spectrometry was used to identify a four gene operon encoded by strain PMC3671E9-1. The proteins were designated as IPD126Aa-1, IPD126Aa-2, IPD126Aa-3 and IPD126Aa-4.

The PMC3671E9-1 DNA fragment containing IPD126Aa-1, IPD126Aa-2, IPD126-3 and IPD126Aa-4 were subcloned into E. coli. The total protein extract from the transformed E. coli cells showed strong activity against WCRW, confirming the insecticidal activity of these proteins.

### Example 5. Identification of Homologous Sequences

Genome sequences analysis indicated homolog sequences in other strains. Pantoea agglomerans strain PMC3671E3-1 contains all four IPD126 genes and have second copy of the fist three genes upstream. Another Pantoea agglomerans strain, PMCJ4082D4-1 (NRRL Deposit No. B-67699), is also active on WCRW and contains two operons, similar to PMC3671E3-1. These corresponding homolog sequences show 70 to 100% amino acid sequence identities to those in PMC3671E9-1.

### Example 6. Live Culture Assay and Results

WCRW bioassays were conducting using live cultures of 20 ul samples and molten artificial diet in a 96 well format. A serial dilution of the overnight PMC3671E3-1 culture was tested on multiple insect targets. The culture and washed pellet showed killing activity against WCRW.

TABLE 1
WCRW Bioactivity
PMC3671E3-1 culture

| Dilution | | SBL | ECB | FAW | VBC | CEW | WCRW |
|---|---|---|---|---|---|---|---|
| 1 | x | 0 | 0 | 0 | 0 | 0 | 3 |
| 1/2 | x | 0 | 0.5 | 0 | 0 | 0 | 3 |
| 1/4 | x | 0 | 0 | 0 | 0.25 | 0 | 3 |
| 1/8 | x | 0 | 0.5 | 0 | 0 | 0 | 3 |
| 1/16 | x | 0 | 0 | 0 | 0 | 0 | 3 |
| 1/32 | x | 0 | 0 | 0 | 0.75 | 0 | 2.5 |
| 1/64 | x | 0 | 0 | 0 | 0 | 0 | 2 |
| 1/128 | x | 0.75 | 0.8 | 0 | 0 | 0 | 0 |

PMC3671E3-1 spent media

| | | | | | | | |
|---|---|---|---|---|---|---|---|
| 1 | x | 0 | 0.75 | 0 | 0 | 0 | 1.5 |
| 1/2 | x | 0 | 0.25 | 0 | 0 | 0 | 1.5 |
| 1/4 | x | 0 | 0.25 | 0 | 0.75 | 0 | 1 |
| 1/8 | x | 0.25 | 0 | 0 | 0 | 0 | 1 |
| 1/16 | x | 0 | 0 | 0 | 0 | 0 | 0 |
| 1/32 | x | 0 | 0.5 | 0 | 0 | 0 | 0 |
| 1/64 | x | 0.25 | 0 | 0 | 0 | 0 | 0 |
| 1/128 | x | 0 | 0 | 0 | 0.75 | 0 | 0 |

PMC3671E3-1 washed cell pellet

| | | | | | | | |
|---|---|---|---|---|---|---|---|
| 1 | x | 0 | 0 | 0 | 0 | 0 | 3 |
| 1/2 | x | 0 | 0.25 | 0 | 0 | 0 | 3 |
| 1/4 | x | 0 | 0.25 | 0 | 0 | 0 | 3 |
| 1/8 | x | 0 | 0 | 0 | 0 | 0 | 3 |
| 1/16 | x | 0.25 | 0 | 0 | 0 | 0 | 3 |
| 1/32 | x | 0 | 0.75 | 0 | 0 | 0 | 2.5 |
| 1/64 | x | 0.25 | 0 | 0 | 0 | 0 | 1.83 |
| 1/128 | x | 0 | 0 | 0 | 0 | 0 | 1 |

Scores: 3-killing; 2-severe stunting; 1-stunting; 1-no activity.

1. A composition comprising an agriculturally acceptable carrier, a plant or a plant part, and a bacterial strain having a 16S rDNA sequence comprising at least 95% sequence identity to any one of SEQ ID NOs: 37-39, wherein the bacterial strain further comprises an IPD126 gene, and wherein the bacterial strain has insecticidal activity.

2. The composition of claim 1, wherein the bacterial strain is a Pantoea agglomerans.

3. The composition of claim 1, wherein the bacterial strain is a Pantoea agglomerans strain PMC3671E3-1 (NRRL Deposit No. B-67697), a Pantoea agglomerans strain PMC3671E9-1 (NRRL Deposit No. B-67698), or a Pantoea agglomerans strain PMCJ4082D4-1 (NRRL Deposit No. B-67699), or a progeny, mutant, or variant thereof.

4. The composition of claim 1, wherein the IPD126 gene comprises a nucleic acid sequence encoding a polypeptide, wherein the polypeptide has at least 90% sequence identity to any one of SEQ ID NOs: 19-36.

5. The composition of claim 4, wherein the nucleic acid sequence comprises at least 90% sequence identity to any one of SEQ ID NOs: 1-18.

6. The composition of claim 1, wherein the composition further comprises a biocontrol agent selected from the group consisting of a bacteria, a fungus, a yeast, a protozoa, a virus, an entomopathogenic nematode, a botanical extract, a protein, a nucleic acid, a secondary metabolite, and an inoculant.

7. The composition of claim 1, wherein the composition further comprises an agrochemically active compound selected from the group consisting of an insecticide, a fungicide, a bactericide, and a nematicide.

8. The composition of claim 1, wherein the composition further comprises a compound selected from the group consisting of a safener, a lipo-chitooligosaccharide, a triglucosamine lipoglycine salt, an isoflavone, and a ryanodine receptor modulator.

9. The composition of claim 1, wherein the plant or the plant part is genetically modified.

10. A fermantate broth, wherein the fermentate broth is produced using a bacterial strain having a 16S rDNA sequence comprising at least 95% sequence identity to any one of SEQ ID NOs: 37-39, wherein the bacterial strain further comprises an IPD126 gene, and wherein the fermentate broth has insecticidal activity.

11. The fermentate broth of claim 10, wherein the bacterial strain is a Pantoea agglomerans.

12. The fermentate broth of claim 11, wherein the bacterial strain is a Pantoea agglomerans strain PMC3671E3-1 (NRRL Deposit No. B-67697), a Pantoea agglomerans strain PMC3671E9-1 (NRRL Deposit No. B-67698), or a Pantoea agglomerans strain PMCJ4082D4-1 (NRRL Deposit No. B-67699), or a progeny, mutant, or variant thereof.

13. The fermentate broth of claim 10, wherein the IPD126 gene comprises a nucleic acid sequence encoding a polypeptide, wherein the polypeptide has at least 90% sequence identity to any one of SEQ ID NOs: 19-36.

14. The fermentate broth of claim 13, wherein the nucleic acid sequence comprises at least 90% sequence identity to any one of SEQ ID NOs: 1-18.

15. A composition comprising the fermentate broth of claim 10, wherein the composition further comprises a biocontrol agent selected from the group consisting of a bacteria, a fungus, a yeast, a protozoa, a virus, an entomopathogenic nematode, a botanical extract, a protein, a nucleic acid, a secondary metabolite, and an innoculant.

16. A composition comprising the fermentate broth of claim 10, wherein the composition further comprises an agrochemically active compound selected from the group consisting of an insecticide, a fungicide, a bactericide, and a nematicide.

17. A composition comprising the fermentate broth of claim 10, wherein the composition further comprises a compound selected from the group consisting of a safener, a lipo-chitooligosaccharide, a triglucosamine lipoglycine salt, an isoflavone, and a ryanodine receptor modulator.

18. A method for controlling a plant pathogen, pest, or insect comprising applying to a plant, a plant part, or an environment of a plant or a plant part bacterial strain or a fermentate broth produced by a bacterial strain, wherein the bacterial strain has a 16S rDNA sequence comprising at least 95% sequence identity to any one of SEQ ID NOs: 37-39, wherein the bacterial strain further comprises an IPD126 gene, and wherein the bacterial strain has insecticidal activity.

19. The method of claim 18, wherein the bacterial strain is a Pantoea agglomerans.

20. The method of claim 18, wherein the bacterial strain is a Pantoea agglomerans strain PMC3671E3-1 (NRRL Deposit No. B-67697), a Pantoea agglomerans strain PMC3671E9-1 (NRRL Deposit No. B-67698), or a Pantoea agglomerans strain PMCJ4082D4-1 (NRRL Deposit No. B-67699), or a progeny, mutant, or variant thereof.

21. The method of claim 18, wherein the IPD126 gene comprises a nucleic acid sequence encoding a polypeptide, wherein the polypeptide has at least 90% sequence identity to any one of SEQ ID NOs: 19-36.

22. The method of claim 21, wherein the nucleic acid sequence comprises at least 90% sequence identity to any one of SEQ ID NOs: 1-18.

23. The method of claim 18, further comprising applying to the plant, plant part, seed, or environment of the plant or plant part with an agriculturally acceptable carrier.

24. The method of claim 18, wherein the composition further comprises applying to the plant, plant part, or environment of the plant or plant part a biocontrol agent selected from the group consisting of a bacteria, a fungus, a yeast, a protozoa, a virus, an entomopathogenic nematode, a botanical extract, a protein, a nucleic acid, a secondary metabolite, and an innoculant.

25. The method of claim 18, wherein the composition further comprises applying to the plant, plant part, or environment of the plant or the plant part an agrochemically active compound selected from the group consisting of an insecticide, a fungicide, a bactericide, and a nematicide.

26. The method of claim 18, wherein the composition further comprises applying to the plant, plant part, or environment of the plant or the plant part a compound selected from the group consisting of a safener, a lipo-chitooligosaccharide, a triglucosamine lipoglycine salt, an isoflavone, and a ryanodine receptor modulator.

27. The method of claim 18, wherein the plant, the plant part, or the environment of the plant or the plant part comprises a genetically modified plant, a genetically modified plant part, or an environment of a genetically modified plant or a genetically modified plant part.

28. A recombinant polynucleotide encoding an insecticidal polypeptide an IPD126 polypeptide comprising an amino acid sequence having greater than 80% sequence identity compared to the amino acid sequence of any one of SEQ ID NOs: 19-36.

29. The recombinant polynucleotide of claim 28, wherein the recombinant polynucleotide comprises the polynucleotide of any one of SEQ ID NOs: 1-18.

30. A DNA construct comprising, the recombinant polynucleotide of claim 28 and a heterologous regulatory sequence operably linked to the recombinant polynucleotide.

31. A transgenic plant or plant cell comprising the DNA construct of claim 30.

32. A method of controlling Lepidoptera and/or Coleoptera insect infestation in a transgenic plant and providing insect resistance management, comprising expressing in the plant the polynucleotide of claim 28.


SEQUENCE LISTING (39 sequences)

SEQ ID NO: 1 - DNA, 7569 bp, Pantoea agglomerans
  source 1..7569: mol_type = genomic DNA
atgtatctgaccgaagaaatacttgccaaactgaatgccggaaacggcaagctacaatctactgtagagcaggaaattacgctgccagatattatggtgcgctcttttgctcaggtaaaagaactggcaggagacaggttaagttggggtgagaaaaacttcctttatcagcaggctcagacacagctgaaagaaaataaaatggcagaatcccgcattctcagccgtgccaacccgcaactggcaaatgctgtccggctgggcatccgccagtcttcgatgctgggtagctatgacgacctgttcccgcagcgcgccagccgctttgttaagccaggtgcggttgcctcaatgttttcaccggctggctatctgaccgagctgtaccgcgaggccagaggattacacaaggctgaatcgcaatataatcttgataaccgccgtccggatctggcctcgctgacgctatcccagtcaaacatggatgacgagttgtccaccctgtcgctctccaatgaactgctgctgaagttaattcagtcaaaggaaagcctgacttatgaacaggttatggaaaagctggcgacttacagactgaccggcaccacgccttacaatcaaccctatgaagcaatccgtcaggctattttgctgcaggacccggagtttaacgcgttcagtaataatccggcagtggccgcaaaaatcaacaccagcgggctattaggcattacttccgatatcgccccggagctgcatgcgatactgactgaagagataacagaagaaaacgcggaagcactggttaaaaagaacttcggcgatgtcaatatcaagcagttccaaaatcttgcgtggctggccaactggtacggcttgtcctatgagaagcttaataacctggtaggcatgatttggtccagagatgatcttgaccccgctattgagcactataaaaattccagcctggtcactttggtggctgaagacggtggatcgcttaacgcggtgttgattaagcgtactaaaggccatgattccgatgatatgcattatgcggaattaattcctgtgggaggagacaaatttcagtacaacttcagccttattgatgctgaagccagtagttcttattatcaattcggtacaaaaggaaagtactcccaagatttagttcctgcaatccataagcctttgctgggtaatactccctatgctgttacattcacacttacacaagagcagctaagtaacccagttgaaatatccctgacgcatggtagtggcggtggtgatcgccttacctcaacaattttcactgttacgacttccccatttgatatcttcctgctgaagctgaataaacttatacgcctctataaagccaccggtatctccccggccagcatcaggaccgtgattgaaagcgataacactgaccttatcatcacagaaagcgtattaagccagctattctggactaattactatacacaaacgttcgaaatggaattttctgccgcactggtgctggcaggagcggacatcggtcagatagcacacagtgaaagccagccaagtgcgttcacccgcctgtttaacacgccgccgctggataaccagcagttttcggccagcgacgagtcactggatctggagccgggtaagggagccgatgctttccggatcgctgtacttaaacgtgcattgcaggtgaatgacgccggactgtataccctttatggtctgagtttcaccgataaagataaaaacggtgagttgattccgttcaccactaaaattgagaacctttctgccctctatcgcacccgactgctggccgacatatttaatatttctgtcactgagctgagcatgctgctgtcggtttcaccttatgccagtcagaaggtggacagcctcaaaggtcaggcactacatcagtttgttactaccctcagcgactatatgcaacggctgaaagcgatgaactggagcgtcagcgatctctacctgatgctgaccaacagctacagcacggtactgtcgccagaaattaaaagcctgatgactaccctgaaaaatggactcagcgagcaggattttaacaacacggatgaaatcgctcagctgaatgcgacggcacctttaatcgccgcagcgatgcagcttgactccacagaaaccgctgcagcactgctggaatggcttaatcaattgcaaccagcagggctgacagtggcaggtttcctgtctcttgtgaatcagacgacacccgaagataaggatgttgtaaaactggtctctttctgccaggtgatggggcagcttgcactgatcgtgcgcaaggcggctctgggctccagcgaaatcatctttgcagttgcgcatccggctatctttaacaaagatgcgaactcactggctcaggatattggcacgctctttgacctgacccagctgcatgcatttctgacagaatgtggtacttatgcctctgaaattcttacctcactgaatgaagggaacctcgacgttagcacagtggcgacggcgctgacgctggacaaaactacactggcgcaggcacttgctcaggtttcagaatctaaggccttttctaactggcacgaactgcgtgatgcacttcagtggacagatgccgccagcattttcaacatcacaccagtggctctgactgcgatggtgaacctgaaattcagtggtgacaacgcttcaccgtatcaggagtgggtaacggtcagcaaagctatgcaggccgggctgaatcagacgcaaagtgctcagctgcaagcttcgctggatgaatccctcggtgcagccgtcagcgcctatgtgattaaaaacagttcgccgtcctgggttaccgatcgcgacaaactttacagctggctgctgattgataaccaggtgtcggcgcaggtcaaaaccacccgcatcgccgaagcgattgccagcgtgcagctgtatgtaaaccgggcattaagcggccttgagaatggccaatcaatcactgacgctgttgataatgccgttaaatccggggtattttttacccgcgactgggatacatacaacaaacgctacagcacctgggccggcgtctctgagttggtttactatccggaaaactatgttgacccgaccctgcgccctggtcagaccggcatgatggatgagatgctgcagacgcttagccagagccagctgacgtccgattcggtggaagacgcgttcaaaacctacatgacccgctttgaagaaatcgctaacctggatattgtcagcggctatcatgacaacctcaatgaccagaagggtgtaacatatctgatcggtcgctccgctgctggcgactattactggcgttcggcagatatcagtaagctttctgacggtaagctcccagctaacgcctgggccgagtggaaaaaaattaccaccgcgctgacgcccgtaaataacctggtgcgcccggtaatatttcagtcacgattgtatgtgacctgggtggaaagccgcgaagtcggcatatccgccgacaaagagcacaacagtgaaaccaaaattctggagtatgctctgaagtatgcacatattctgcatgacggtacctggagcgcacccgtgtctgttaagcttgagaacggaacgctgcctcttgacagcgtggctattgatgttacaggcatgtattgcgcaaaggatacacagcatgaccagctttatattttattttataagaaaaaggaaacttacaatgacgtcaatgacgttctgaaaggaataatactgcacgatgacgggactaccaccattacttccggtaatagcgtatctggattggttgtctataaacaactggatactactaaggaagtcaggctgaatacgccttacccgggaggaaaaacatactactctattaataatatgagggaatcgagtaaatggggagatgataatatttcaatgctgtcaggatgtagcgtgaaagattttgtctttaccgaaggcgatgggaaactgaatgttgcgttcaatgccaccgaacgcattatataccgtggtaatccggatagtcagggctatgtggccctggtcaatatgattaaagctatcggaaatattggagacacctttaaaattccggttttgaattcaaatggagagggcttagacaaaccttttacatgtatattcagacagcctgatgaaaagactgatgcgattgcttatttctccgatgtccagggattaaatatagatcatttcgctttcaacgatgaaagtcagaaaatgctgggtcgtatcttaaggcctgaagagaaagatttttataaattagagtgtgtcaatactaatctccatatatacaaagacagtagcaaaacaatcaaaccggataacttcgtgtattttggcccaggcatggatcttatcgtagttaaaggaatgatcgtggaaaccctttttggattatttggagagcttaaaaccggaataaaagataagagtgtgaaactatccgtttctgccggagtcattgacaattcaccagccgctacgaagactaagtatacattcgacgaatcgctgtatgttattgaaggccaaaccgtttctattcaacttagtgaatttaaagaaaataatattgaccttgaattcactttctttgcttctggagacagtgggaactcactaggcaaaagtgtcatcagcgcaacattgacccgaacgagtgaaaacactatacccgttatttctctgaataaaacctctgacaacgcgcagtatttgcagtatggcattcatcgcataagggtgaatacgctgtttgcaaaacagctggttgcgcgcgcgaacgccggactggacactgtactgtctatggcaacccagcagctgacggaacctaaaatgggcaaaggtgcgtacattgaccttgaacttaatgccagcagcgatggcagttcggcggtatttgaagtattgatgtgtgacgtttttaccaaaggtgaccgcattgctttgaccagcggcacactcagccccacagcacgcaccagctgctcatttttcattccccgactgggtgagtctactgaatctccatggaatatgtatttttgcgtaaaaactcagaatgatgagagtaagcgggtagaagtaatgggaggcgaggggaaatggagttaccagtacgtcgatgaatctggtactgccattaagccgccctataccgatccatatattgcaagcgtatatgtacggaacgatacaacagagccgatggacttcaacggtgccaatgcgctctatttctgggaaatgttctattacgtaccgatgatggtatttaagcgcctgctaagcgaaagtaagttcgctgaagcgacccagtggatcaaatatatctggaatcccgatggctatctggtgaacaatcagcccgcgacctatagctggaatgtgcgcccgctggaggaggatacctcctggcacgctgacccgctagactcggttaacccggatgctgtcgcgcaggccgatccgctgcactacaaggttgctacttttatggcgtaccttgatctgctgattgcccgcggtgatgctgcctatcgccagctggagcgcgataccctgaatgaagccaaaatgtggtacgtgcaggcgctgaacatccttggcgatgaaccctaccagtcatcatccagcggctggagctcgccagttctctccagcgcagcagctcagactacagagaaaaacgttcagcaggcgatgctggcggtgcgtcagcagcctgacgcaggagaactgcgcaccgccaactcgctgactgacctgttcctgccccagcagaacgcgaagctggctggttactggcagacgctggcgcagcgcctgtataacctgcgccacaacctgtcaattgacggcagcccattgtcactggccatctatgccgcgccagccgatccggcagcgctgctcagcgcggcggtcaacagcgcgtccggcggcagcgacctgcctgctgttgttatgccgctgtaccgcttcccggtcattctggagagcgcccgggggatggcaggtcaactgatccagttcggcagcacgctgctcagcattgctgaacggcaggatgcggaagcgttgtcggagctgatgcagactcagggtagtcaactgattttgcagagcatcgcactgcaaaacagtacgatttctgaaattgatgcggataaaaccgtgctggaagcgagcctaagcggtgcacgttcgcgcctcgaccggtacaccacgctgtatgacgaggatgtaaatacgggggaacagcaggctatggatctgttctacgcctcctccctccaggcaaacggcggccagatgttccacactatcgcaggcgcacttgacctggtgcctaacatctttggtctggccgacggcggttcgcgctggggtgcggtatctactgcaatggccagcatcgccgatttgtccgccgcagcctgtcacacgaccgcagagcgcctcagccagtctgaggtctaccgccgccgccgccaggagtgggaaatccagcgcaacgccgcgcagtctgaaattgaccagattgacgctcagctggcctcactgacgatacgtcgcgagggcgcggtactgcagaaaacctacctggaaacgcagcagggtcagatgcaggcgcagatgactttcctgcagaataagttcaccagcaaggcgctgtacaactggctgcgcggcaagctggcggccatttactatcagttctacgacctgacggtatcgcgctgcctgatggcagaagccgcctatagctgggatattaaaggtaatcaggaaacaggtacctttatccgtcccggcgcctggcagggcacctatgccggcctgatggcaggggaaacgctgatgctgaatctggcacagatggaaaacagctacctgacaaaagatgagcgcctgaaagaggtcacgcgcacggtctgcctgtctgaagtttatgcagggctctcttcggattcgttcgcgctggctgataccgtcaccacactggtgagtaacgggaaaggcaacgccggcacggacgataacggagtgaagatcgatgacaagcagcttctggctaccctgaaactctccgatctgagcattgataacgattatccggagtcactgggcaaaacccgacgcattaagcaaatcagcgtgacgctgccgacgctggtcgggccgtatcaggacgtccgggcggtactgagctatggcggcagtgtggctctgccacgcggctgcacggcggtcgccgtttcgcacggcatgaatgacagcggccagttccagctggacttcaatgacagccgctggttgccgtttgaaggtatacctgtcggggattccggtacgctgacgctcagcttcccggacattaccgataaacagcaggaaaatctgctgctcagtctgagcgacatcatcctgcacatccgctataccatcgcaagctga

SEQ ID NO: 2 - DNA, 4266 bp, Pantoea agglomerans
  source 1..4266: mol_type = genomic DNA
atgcaaaatacagatcagatgagcctgacgcccccttccttaccctcaggtgggggtgccgtcaccggactgaaaggcgatatgtcaggagccggccccgatggcgccgccacgctgaaccttcccttgccgatcagccccggacgtggctatgcgccgtcgttgtctctgggttaccacagtcgtaacggcaacggtgtttttggcgcaggctggagctgcggtcagatggctattcgcctgcaaacccgcaaaggcgtgccgttttatgacggcagcgacgtctttaccgctcctgacggtgaggttctggtgccggcgctggacgccagcggcaagactgaggttcgcacgaccactacgctgctcggcgaaaacctcggcggcacctttaccgtacagacctaccgttcccgggtggaaaccgacttcagtcgcctggagcgctgggtttcgcaggccgacgcagcggctgatttctggttgatttatagtccggacggccagatccacctgctgggtcgtaacccgcaggcgcgggtgagcaaccctgaggatacaacccagaccgccgcctggctgatagagtcgtcggtctctgccagcggcgagcagatttactggcaataccggcaggaagatgagctgggctgtacgcaggatgagaaaacggctcatgcacacgcgctcgcccagcgctatctggtggcggtatggtatggcaataaagcggccagccggacgctgccggggctgctgtctgttcctgcggctggcagctggctgtttacgctggtgctggactacggtgagcggacgacagatcctgcaacgccaccggcctggctttcaccaggcagtggtacatggctctgccggcaggatgtgttttccagctgggaatatggctttgagctgcgcacgcgtcgcctgtgccgacaggtactgatgtatcatgacgtcgcggcgctggcaggtaagcctggttcagatgccgtgccacagctggtcaccagactgctgctggactataacctgtctccgtcgctgactaccctgaaaaccgcacagcaggccgcctgggaagcggatgggacgttgcgcagtctgccgccgctggcgttcagctggcagaccttcccgtcaacaccagagaaaagtgtcagctggcagcagcggaatgacatggggaaactcaacccacagcagccttatcagatggttgacctgcacggtgaaggactggcgggtatcctctaccaggacagtggtgcctggtggtatcgggagccggttcgtcagttgggcgatgatgataatgccgtgacctgggcggctgcccgaccgctgccggcgttccctgctctgcgcaagggcggaatgctgctggatctggacggtgatggctacctggaatgggtggtcaccgcgccaggcgtcgcgggctgctatgcgcaaacgcctgaacaatgctggcagcgcttcacgccgctgtctgcactgcctgtggaataccgccactcgcgaatggagataaccgacgtcaccggtgcgggtcttgcggatatgctgctgatcggcccgaaaagcgtacgcctgtacagcggcagcggcagaggctggaaaaaagcacgaacggtcatgcaggacagtggcatcaccctgccggtccccggcacaaacgcccgtgtcatggtggcattcagcgatatggccggcagcggtcagcaacacctgacggaaatcaaagccagcggcgtacgttactggcccagcctggggcatggtcgctttgcggcgccggtgacactccccggcttcagtcagcccgcagaaaccttcaacccggcacagctctatctggccgacgttgacggttccggtaccaccgatctgatctatgctctgagcgatcatctgctggtatggctaaaccagagcggaaaccgctttgacgagcctttccgcatcgaccttccggaaggcgtgcgctatgacaatacctgcagcttgcaggtcgccgatattcaggggctgggcatctccagcctggtgctgtcggtgccacatccgacgccgcgccattgggtatgtcacctgacggcggaaaagccctggctgctcgacggcatgaacaacaatatgggtgcccgccatactctctgttaccgtagttcggcgcagttctggctggatgaaaaggctgctgctactgccgatcggcccgcgccggcgtgttatatgccgtttgcgttgcatacactgagccgtactgaagtcagtgatgaaattaccggaaaccggctcaccaggacgatacgctaccggcacggcgtctgggacaggcgcgagcgagagttccgcggctttggctttgttgaagtcagtgatgccgaagcgctggcaaaacaaactgaagggatgagcgcaccggcagttaaacgcagctggtatgctaccggactgacagccgtggatgcacagctcccggatgagttctggaaaggcgatcatgcggcctttgccggttttacccctcgttttaccaccggctatggtgaacaagaggcggcactggataccatcagcgacgatacccgtttctggctgacccgggcgattcgcggtacgctgctgcgtagcgaactgtatggcgcggatggcagcagccaggccgggatcccttacagcatcacggaatcgcggccacaagtgcggttgattactgaggcgggtaattcgccggtggtctggccctccgttatcgagaaccgcgccagtcattatgagcgcgtcagcagcgatccgcagtgcggccagcagatcctgttaaccagtaatgaatatggtcagccgcttcgacagatcggcgtcagttatccccggcgcaccaggcccgatgccagcccctacccggacgatctgccggacggactgtttgccgacagctttgatgagcaacagcaggcgttacgcctgacgctgacacaaagcagctggcatacgctgaaagatatcagcagcggcatctggctgccggccgtggctgatgcaacccgaagcgatctgttcgttcaccaggccgcgcaggtgccgccagcgggtcttacgctggagaatttactcaccgatagtgcgctgctgaccagcccggtttttggtggacagtcgcaaacctggtatcaggacagcgcgggtcaggcgagcaccacctcacccgattttcccctccgaccgtcctttagcgaaaccgcagcgctggacgaggcacaggtcagcgcgctgtcagccgatattgatcaaacgaagctggagcaggcgggctatacccgctcagcgtatctgtttgcacgcagcggtgaggagggtaaaaccctgtggacagtgcgccagggatatatcaccttcagcagcgcagaccatttttatctgccgattgccgcacagcagacgctgctgaccggtaaaaccacggtcacctatgatccgtacgactgtgttgtcttacaggcaaaggatgccgcaggtgcagttacctccgcgacatacgactggcgttttctcgcgccgacgcagattactgatatcaacgataatctgaaaagcgtcacgctggatgcgctgggtcgggtaacgtcgcagcgtttcagcggcagtgaaaacggaaaaccggcgggctacagcgatgatgcgtttccactgccggccagcgccgatgcagcgctggcgctcagtgccccgctaccggtggcacagtgcatcatctacgtaccggacagctggatgctgaccggtgagcagcagcagccgccgcacgtggtaacgctgctcaccgaccgttacgacagcgacagtcagcagcagatccgacagcaggttgttttcagcgatggttttggccgggtgctgcaggctgcctcaaggcaggtgaacggcgaagcgtggcagcgggcggcaaacggctcgttcgttgccgacccgaacggttcgcccgtgctgacggagaccacgttccgctgggctgtcaccggacgcactgaatatgacaataagggacaggccattcgtacttatcagccatattttctggacagctggaaatacgtgcgtgacgacagcgcgcgacacgatctgtacgccgacacccactattacgatccggtggggcgggagcggcaggtcattaccgcaaaaggtttgctgcggcgtgtcacttacaccccctggttcgtagtcagcgaagacgaaaacgatacgcaggcgtag

SEQ ID NO: 3 - DNA, 3015 bp, Pantoea agglomerans
  source 1..3015: mol_type = genomic DNA
atgtccgccgcgtatgtcttaagtaatttatcttatcaacgggagaatactatgagcacctcgctttacagcaggaccccctcagtcacggtcctcgacaaccgcggcctgaccgtacgcgatatcgcgtaccaccgccatccggataccccggcggtgaccagtgaacgcatcacccgccatcagtacgatgcccgcggctttctgacgcagagcgccgacccgcgcctgcacgacgccgggctggcgaacttcagctaccggaccgacctgaccggcagcgttctccgcctacagggcgtcgataacggcatcaccgtggcgctgaacgatgccgccggacggccgtttctggcggtcagcaacatccgcacggccggtgatggctcggaggacagaagccaggcactgacccgtacgtgtcagtacgaggacgcaaccctgcccggacgtccgttaagtattacggagcaggtaaagggtggagccgcccgcatcacggagcgcttcatctatgccggtaacgctgttgaggagaaagccctgaacctcgccgggcagcccgtcagccactatgataccgccggtctgacacagacagacagcatcgccctgaccggcgtgccgctctccgtcacccgccgcctgctgaaggacgcagacaatcctgacgccgtggctgactggcagggaacagacgcctccgtctggaacgacccgctcgacgtggaaacatacactaccctgtccacggcagacgccaccggcgcggtgctgaccaccaccgatgcgaagggaaacctgcagcggctggcctacgacgtggcgggcctgttgtcgggcagctggctgacgctgaaggatggcacggagcaggttatcgtgacgtccctgacctactccgccgccgggcagaagctgcgcgaggagcacggcaacggcgtggtgaccacctacacgtatgaagccgagacgcagcgcctgaccggcattaaaacggcgcggccggccggacacacctcaggtgcgaaggtgctgcaggacctgcgctacacctatgacccggtgggcaacgtcctgaaaatcagcaacgatgccgaagagacccgcttctggcgtaaccagaaagtggtgccggagagcgcgtacgtttatgacagcctgtaccagctggtcagcgccaccggacgcgagatggcgaacgccggtcagcagggcagcagctcatcgtcagccaccgtcccccttcccgccgacagttccgcgtttacaaactatacccgcaactatacttacgatgaggccggcaacctgacgcaggtccgtcataccccggctacgggcagcggctacaccacaaaaataaccgtctctgataaaagcaaccggggtgtgctgagcacgctgacggaaaatccctccgacgttgacgcgctgttcacggcgggcggccagcagaaacagctgcagccggggcagagtctcatctggacgccgcgtaacgagctgctgaaggtgacgccggtagcacgtgacggcggtgcggatgacagcgaaagctaccgctacgacgggggcagcctgcggctgctgaaggtcagcgtgcagaaaaccgggaacagcacgcagacgcagcgggcgctgtacctgccaggactggagctgcgcaacacaacatccggtgatacggaaacggagagcctgcaggtggttaccgtgggtgaagcggggcgcgcgcaggtgcgggtgctgcactgggagagcggaacgccggacagtgtcagcaacgaccagctacgctacagctacgataacctgaccggcagcagcgggcttgagctggacagcagcggcaatattatcagcatggaggaatactatccgtacggcggcacggcggtctggacggcgcgcagcgcggtggaggcgaagtacaaaaccgtgcgctactcggccaaggagcgtgacgccacggggctgtactactacgggtaccggtactaccagccgtgggctggccgctggctgagcgcggacccggcgggcacggcggacgggctgaacctgttcaggatggtaagaaataatccggttacgcttaaggacacaaacgggttgatcagtacgggtcaggatgcccggaaattagtggccgaagcatttgttcaccctttgcatatgactgtctttgaaagaatttcttcagaagaaaatcttgcaatgagcgtgagagaggctggcatttatactatttcggcactgggtgaaggtgctgcagcaaaagggcataatattcttgagaagaccattaaacctggttcattaaaggctgtttatggtgataacgccgaatccattcttgcgcaggcaaaacgcagcggttttgttggccgggtaggtcagtgggatgcatccggtgtacggggaatttatgcacacaacacaccaggtggcgaagacctggcctatccagtcaacttaaaaaatagttctgctaatgaacttgttaatgcatggataaaatttaaaatcatcacgccttataccggtgattatgacatgcacgatattattaaaatctcggatggaaaagggcatgtgcccctggcggaaagtaatgaggaaaaaggtgtaaaggatatgattaatgaaggtgttgcgcaggtcgaccctgccagaccctttacgtctacagcgatgaatgttgttcgccatggccctcaggtaaactttgttccctatatgtgggaacatgagcacgaaaatgtcgtaagggataatggttatctgggagtggtagctcgtccgggtccattccctgttgcgatggtacataagggtgaatggactgttttcgacaataaaaacgagctgtttgagttttataaatctacaaacactcctcttcccgaacactggtctcaggattttgttgagagagggaaaggaaatgttgcaacgccccgacacgctgaaattcttgatcgtaattcctcgcgtctaagagcggcctga

SEQ ID NO: 4 - DNA, 2913 bp, Pantoea agglomerans
  source 1..2913: mol_type = genomic DNA
atgtgtagcgttgccgattttgatcggctgcacaacataaaacaggagaatatcatgagcacctcgctttacagcaggaccccctcggtcacggtcctcgacaaccgcggcctgaccgtacgcgatatcgcgtaccaccgccatccggataccccggcggtgaccagtgaacgcatcacccgccatcagtacgatgcccgcggctttctgacgcagagcgccgacccgcgcctgcacgacgccgggctggcgaacttcagctaccggaccgacctgaccggcagcgttctccgcctacagggcgtcgataacggcatcaccgtggcgctgaacgatgccgccggacggccgtttctggcggtcagcaacatccgcacggccggtgatggctcggaggacagaagccaggcactgacccgtacgtgtcagtacgaggacgcaaccctgcccggacgtccgttaagtattacggagcaggtaaagggtggagccgcccgcatcacggagcgcttcatctatgccggtaacgctgttgaggagaaagccctgaacctcgccgggcagcccgtcagccactatgataccgccggcctgacacagacagacagcatcgccctgaccggcgtgccgctctccgtcacccgccgcctgctgaaggacgcggacaatcctgacgccgtggctgactggcagggaacagacgcctccgtctggaacgacctgctcggcgcggaaacatacaccaccctgtccacggcagacgtcaccggcgcggtgctgaccaccaccgatgcgaagggaaacctgcagcggctggcctacgacgtggcgggcctgctgtcaggcagctggctgacgctgaaggatggcacggagcaggttatcgtgacgtccctgacctactccgccgccgggcagaagctgcgcgaggagcacggcaacggcgtggtgaccacctacacgtatgaagccgaaacgcagcgcctgaccggcattaaaacggagcggccggccggacacgcctcaggtgcgaaggtgctgcaggacctgcgctacacctatgacccggtgggcaacgtcctgaaaatcagcaacgacgccgaagagacccgcttctggcgcaaccagaaagtggtgccggagaacgcgtacgtttatgacagcctgtaccagctggtcagcgccaccggacgcgagatggcgaacgccggtcagcagggcagcagctcatcgtcagccaccgtcccccttcctgccgacagttccgcgtttacaaactatacccgcaactatacttacgatgaggccggcaacctgacgcaggtccgccataccccggctacgggcagcggctacaccacaaaaataaccgtctctgataaaagcaaccggggtgtgctgagcacgctgacggaaaatccctccgacgttgacgcgctgttcacggcgggcggccagcagaaacagctgcagccggggcagagtcttatctggacgccgcgcaacgagctgctgaaggtgatgccgataatgcgtgacggcggtacggatgacagcgaaagctaccgctacgacgggggcagccagcggctgctgaaggtcagcgtgcagaaaaccggcaacagcacgcagacgcagcgggcgctgtacctgccgggactggagctgcggacgacaaaatctggcgatacgctaacagaaagcctgcaggtgattaccgcgggcgaagcgggccgggcgcaggtgcgggtgctgcactgggagagcggaacgccggacagtgtcagcaacgaccagctacgctacagctacgataacctgaccggcagcagcgggcttgagctggacagcagcggcaatattatcagtatggaggaatactatccgtacggcggcacggcggtctggacggcgcgcagcgcagtggaggcgaagtacaaaaccgtgcgctactcgggcaaggagcgtgacgccacgggactgtattactacggataccggtattatcagccgtgggcgggcagatggctgagtgcggacccggcgggcacggtggacgggctgaacctgttcagaatggtgcgcaataatccgctcacattaaaagataacgatgggctaaaaccaataaatgaaaatttcagagaaaataaaggcgatctggtttatgggctggctgccccccgaggagcttatatatcaacggcaatagggcgcaaattcgctccagaagagaaagatgcacctgcttcaattattgacttatataacaatacggtttccggacaagcccttcttagcgttgatttcaaaatactgcaagacttcatgaagtcaccaaaaaaaaatgaaaaaaaactagcccctccatctaacattaaagagttggtaaaaaaatccagggattatcctctgtgggaggattattttctggcaggggaaaacaaccccaaatttaacattgcatccatatataaagaggtcagaaaagatgcaggaaagacccagtatcatgagtggcatatagcgggaggacaatcggcacctaagctactttggaaacgaggaagtaagttgggtattgaaatggcggccagcggtgctggcaataaaatccattttgttcttgatgaactggatatatcaaatgttgtaaataaggaagggccgggggggaaatctataacggccagtgagttacgttatgcgtacagaaatcgtgaaagactgactggaaatatacatttttacaaaaataatgctgaaactggcgcaccatgggatacaaatgctgaactgtgggcatcttatcaccccaaaccaaagcataaaggaaatgaatcgacacacataatgtcgcaaaggagaaatggtagtctgttcaaatccatgagaaaggttttctcaagaaat

SEQ ID NO: 5 - DNA, 7572 bp, Pantoea agglomerans
  source 1..7572: mol_type = genomic DNA
atgtatctgaccgaagaaatacttgccaaactgaatgccggaaacggcaaactacaatctactgtagagcagataattacgctgccagatattatggtgcgctctttttctcaggtaaaagagctggcaggagacaagctaagttggggtgagaaaaactttctttatcagcaggctcagacacagctgaaagaaaataaaatggcggaatcccgcattctcagccgtgccaacccgcaactggcaaatgctgtccggctgggcatccgtcagtcttcgatgctgggtagctatgacgacctgttcccgcagcgcgccagccgctttgttaagccaggtgcggtggcctcaatgttttcaccggctggctatctgaccgagctgtaccgtgaggccagaggattacacaaggctgaatcgcaatataatctcgataaacgccgtccggatctggcctcgctggcgctatcccagtcaaacatggatgacgagttgtccactctgtcactgtcaaatgagctgctgctgaataatatccagcagcatgatggcctcagctatgacgatgcgctgaagaagctcgccggataccgacaaaccgggacaacaccctatagccagccgtatgaaacgatccgcgaggctattcttctgcaggatcctgcgtttaattcgattcgcaacaatccagctgtggcgactaaaatgaataccagtggcctgctgggactgacagcaaacctgccacctgaactgcatgcgatcctgactgaaacgattaccgaagagaatgcggaacaactgattaaggacaatttcggtgatgtcaacgtcagcagatttcaagatgttagttatcttgcccgctggtatgggatgaccccttatgaactgaactcggtgctgggactgatggaggtgggcagcaatcctgtcgacggtgttacgtattaccaggatgaccagctgatttccctggtggataatggcggtaatctggatgctgtgctgatgcaacgtgcgggcggtgacaattatagccagtttggttatatcgaactgctaccggtgtcaggggatacctaccagcttcggtttaccgtacaaagtggttacgtgggtcaggactcagaagtccgcattggaacttcagagaatgccggaagcaaggatattcttagcgatggtcgcatcgccggacttaacatccctatggtcctgaacgttaaacttgacagcactaagttggcccaggggatcactatcggcgtaacccgatacgaccccagcggtagttatataaattttgcgtccgttcgtttccagagatacgacttttcttataatgtcttcctgctcaaattaaacaaaattattcgcctctacaaagccaccggcatttcgccatccgatatccagaccctgattgaaagtgctaatcatgacctggctattaccgaagatgtactaagccagctcttctggacgaactattatacgcagcgttacggtattgatttttctgctgcgctggtgctggcgggtgcgaacatcagtcagattgcccacagcaataaacagagcgcgttcacccgcttgtttaacactccgccgctgaacaatcagtttttttatgctgatgggaaaaagctgaacctcgaacccggtaagtcggatgactcacacggtcttggggtactcaaacgggcattacaggttaatgatagcgccctgtataccctttttaatctgacctttgcggataaagacgcccagggtaatgctgttgttttcaccaaaactcctgaaaatctttctgcgctgtaccgtaccagattgctggcgacggttaacaacctgaccgttaacgaactgagcctgctgctttccgtttcaccatacgtaaaggtgaagctggctacgctaaaagacgaggcgctcagccagctgtcgactaccctggaaaggtatacgcaatggctggacaagatgaactggacgatcggcgatctgtacctgatgctgacccctgtttacagcaccgttctgtcgccggatattgaaaatctggtgacgacgctgaaaaatggcctggcgggtcaggacttaaccagcgatgaaaaacgcatcgctgcgctggctccgtttgttgctgccgccactcagctggactctgcggagacggccagggcgcttttacgatggctgaacgatcttaaaccgggaaccctgtcattagcggattttatcgcgcaggttaacaacaccacccaaaccgaaaatctggtcacattcagccaggtgatggcgcagctcgcccttattacgcgcaacgccagcctgagtgcaaatgagctgtcatgggcggttgcgcacccggaaattttccaggaaaaggccactgttcttaaaaacgacatcgccactctcaatgatctgacgcagctgcatgatctgctggcgcgctgtggcagccatgcctcagagatccttacctcgctgagtgggaatgccagcaaggctgaaaataaccttgccgtcagcaccctggcgacggcgttaaatctggacgagcgggcactgacccaggcgctggcgaaggtttccacctatgaatatttttataattgggcacacttaaatgaagcgctgcagtggctggacgttgccaccacctttggcatcacgcccgataacctggccgcgctgattgggctgaagttcgataatcaggatgacgcctcgtttgccagctggcttaccgcaagccggtttatgcaggcagggcttaacacgcagcagacagctcagctgtccgccactctggatgaatccctcagcgcagcggtcagcgcctacgcgatcaaaaatattttctccggcgcggtaagtaacagggagcagctctacagctggctgctgattgataaccaggtgtcggcgcaggtcaaaaccacccgcatcgccgaagcgattgccagcgtgcagctgtatgtaaaccgggcattaagcggccttgagaatggccaatcagccactgacgctgttgataatgccgttaaatccggggtattttttacccgcgactgggatacatacaacaaacgctacagcacctgggccggcgtctctgagctggtttactatccggaaaactatgttgacccgaccctgcgccttggtcagaccggcatgatggatgagatgctgcagacgctcagccagagccagctgacgtccgatacggtggaagacgcgttcaaaacctacatgacccgctttgaagaaatcgctaacctggatattgtcagcggctatcatgacaacctcagcgaccagaagggtgtaacatatctgatcggtcgctccgctgctggcgactattattggcgttcggcagatatcagtaagctttctgacggtaagctcccggctaacgcctgggccgagtggaaaaaaattaccaccgcgctgacgcccgtaaataacctggtgcgcccggtaatatttcagtcacgattgtatgtgacctgggtggaaagccgcgaggtcggcatatccgccgtcaaaaagcaaaacagtgaaaccaaacctctggagtatgctctgaagtatgcacatattctgcatgacggtacctggagcgcacccgtgtctgttaagcttgagaacggaacgctgcctcttgacagcgtggctattgatgttacaggcatgtattgcgcaaaggatacacagcatgaccagctttatattttattttataagaaaaaggaaacttacaatgacgtcaatgacgttctgaaaggaataatactgcacgatgacgggactaccaccattacttccggtaatagcgtatctggattggttgtctataaacaactggatactactaaggaagtcaggctgaatacgccttacccgggaggaaaaacatactactctattaataatatgagggaatcgagtaaatggggagatgataatatttcaatgctgtcaggatgtagcgtgaaagattttgtctttaccgaaggcgatgggaaactgaatgttgcgttcaatgccaccgaacgcattatataccgtggtaatccggatagtcagggctatgtggccctggtcaatatgattaaagctatcggaaatattggagacacctttaaaattccggttttgaattcaaatggagagggcttagacagaccttttacatgtatattcagacagcctgatgaaaagactgatgcgattgcttatttctccgatgtccagggattaaatatagatcatttcgctttcaacgatgaaagtcagaaaatgctgggtcgtatcttaaggcctgaagagaaagatttttataaattagagtgtgtcaatactaatctccatatatacaaagacagtagcaaaacaatcaaaccggataacttcgtgtattttggcccaggcatggatcttatcgtagttaaaggaatgatcgtggaaaccctttttggattatttggagagcttaaaaccggaataaaagataagagtgtgaaactatccgtttctgccggagtcattgacaattcaccagccgctacgaagactaagtatacattcgacgaatcgctgtatgttattgaaggccaaaccgtttctattcaacttagtgaatttaaagaaaataatattgaccttgaattcactttcttggcttctggagacagtgggaactcactaggccaaagtgtcatcagcgcaacattgacccgaacgagtgaaaacactatacccgttatttctctgaataaaacctctgacaacgcgcagtatttgcagtatggcattcatcgcataagggtgaatacgctgtttgcaaaacagctggttgcgcgcgcgaacgccggactggacactgtactgtctatggcaacccagcagctgacggaacctaaaatgggcaaaggtgcgtacattgaccttgaacttaatgccagcagcgatggcagttcggcggtatttgaagtattgatgtgtgacgtttttaccaacggtgaccgcattgctttgaccagcggcacactcagccccacagcacgcaccagctgctcatttttcgtgccccgactggatgagtctactgcatctgcatataatatgtacttttgcgtaaaaactcagaatactgagagtaagcgggtagaagtaacgggaggcgaggggaaatgggattaccagtacgtcgatgaatctggtgctgccattaagccgccctataccgatccatatattgcaagcatatatgtacggaacgatacaacagagccgatggacttcaacggtgccaatgcgctctatttctgggaaatgttctattacgtgccgatgatggtatttaagcgcctgctaagcgaaagtaagttcgctgaagcgacccagtggatcaaatatatctggaatcccgatggctatctggtgaacaatcagcccgcgacctatacctggaatgtgcgccctctggaggaggatacctcctggcacgctgacccgctagactcggttaacccggatgctgtcgcgcaggccgatccgctgcactacaaggttgctacctttatggcgtaccttgatctgctgattgcccgcggtgatgccgcctatcgccagctgcagcgcgacaccctgaatgaagcaaaaatgtggtacgtgcaggcgctgaacatccttggcgatgaaccctaccagtcatcatccagcgactggagctcgccagttctctccagcgcagcagatcagactacagagaaaaacgttcagcaggcgatgctggcggtgcgtcagcagcctgacgcaggagaactgcgcaccgccaattctctgaccagtctgttcctgccgcagcagaatgaaaagctggctggttactggcagacgctggcgcagcgcctgtataacctgcgccacaacctgtcaattgacggcagcccattgtcactggccatctatgccgcgccagccgatccggcagcgctgctcagcgcggcggtcaacagcgcgtccggcggcagcgaactgcctgctgctgttatgccgctgtaccgcttcccgattattctggagagcgcccgggggatggcaggtcaactgacccagttcggcagcacgctgctcagcattgctgaacggcaggatgcggaagccttgtcggagctgatgcagactcagggtagtcaactgattttgcagagcatcgccctgcaaaacagtacgatttctgaaattgatgcggataaaaccgtgctggaagcgagcctaagcggtgcacgttcgcgcctcgaccgatacaccacgctgtatgacgaggatgtaaatacgggggaacagcaggctatggatctgttctacgcctcctctatccaggcaaacggcggccaggcgttccacactgtcgcaggcggacttgacctggcgcctaacatctttggtctggctgacggcggttcgcgctggggtgcagcatttactgcattggccagcatcgccgatttgtccgccgcagcctctcacacggccgcagagcgcctcagccagtctgaggtctaccgccgccgccgccaggagtgggaaatccagcgcaacgccgcgcagtctgaaattgaccagattgacgctcagctggcctcactgacgatacgtcgcaaaggcgcggtactgcagaaaacctacctggaaactcagcagggtcagatgcaggcgcagatgaccttcctgcagaataagttcaccagcaaggcgctgtacaactggctgcgcggcaagctggcggccatctactatcagttctacgacctgacggtatcgcgctgcctgatggcagaagccgcctatagctggcatattaaaggtaatcaggaaacaggtacctttatccgtcccggcgcctggcagggaatctatgccggcctgatggcaggggaagcgctgatgctgaatctggcacagatggaaaacagctacctgacaaaagatgagcgcctgcaagaggtcacgcgcacggtctgcctgtctgaattttattcagggctctcttcgaataagttcgcgctggctgataccgttaccacactggtgaatagcgggaaaggcaacgccggcacgaccgataacggagtgaagatcgatggcaagcagcttctggctaccctgaaactctccgatctgaacattaagacggattatccagagtcactggacaaagccaaacgcattaagcaaatcagcgtgacgctgccgatgctggtcgggccgtatcaggacgtccgggcggtactgagctatggcggcagcgtggttctgccacgcggctgcacggcggtcgccgtttcgcacggcatgaacgacagcggccagttccagctggacttcaatgacagccgctggctgccttttgaaggtatacctgttgatgattccggtacgctgacgctcagcttcccggacattaccgataagcaacaggaaaatctgctgctcagtctgagcgacatcatcctgcacatccgctataccatcgcaagctga

SEQ ID NO: 6 - DNA, 4266 bp, Pantoea agglomerans
  source 1..4266: mol_type = genomic DNA
atgcaaaatacagatcagatgagcctgacgcccccttccttaccctcaggtgggggtgccgtcaccggactgaaaggcgatatgtcaggagccggacccgatggcgccgccacgctgagccttcccctgccgatcagccccggacgtggctatgccccgtcgctgtcactgggttaccacagtcgtaacggcaacggtgtttttggcgcaggctggagctgcggtcagatggctattcgcctccaaacccgcaaaggcgtgccgttttatgacggcagcgacgtctttaccgctcctgatggtgaggttctggtgccggcgctggacgccagcggcaaggctgaggttcgcacgaccactacgctgctcggcgaaaacctcggcggcacctttaccgtacagacctaccgttcccgagtggaaaccgacttcagtcgcctggagcgctgggttccgcagaccgacgcagcggctgatttctggttgatttatagtccggacggccagatccacctgctgggtcgtaacccgcaggcgcgggtgaacaaccctgaggatacaacccagaccgccgcctggctgatagagtcgtcggtctccgccagcggcgagcagatttactggcaataccggcaggaagatgagctgggctgtacgcaggatgagaaaacggctcacgcacacgcgctcgcccagcgctatctggtggcggtatggtatggcaataaagcggccagccggacgctgccggggctgctgtctgttcctgcggctggcagctggctgtttacgctggtgctggactacggtgagcgggcgacagatcctgcaacaccaccggcctggctgtcaccgggcagtggcacatggctctgccggcaggatgtgttctccagctgggaatatggctttgagctgcgcacgcgtcgcctgtgccgacaggtactgatgtatcatgacgtcgcggcgctggcaggtcagtcaggttcagatgccgtgccacagctggtcaccagactgctgctggactataacacgtctccgtcgctgactaccctgaaaaccgcacagcaggccgcctgggaaccggatgggacgttgcgcagtctgccgccgctggcgttcagctggcagaccttcccgtcaacaccagagaaaagtgtcagctggcagcggcggaatgacatggggaaactcaacccacagcagccttatcagatggttgacctgcacggtgaaggactggcgggtatcctctatcaggacagtggtgcctggtggtatcgggagccggttcgtcagtcgggtgatgatgataatgccgtgacctgggcggctgcccgaccgctgccggcgttccctgctctgcgcaagggcggaatgctgctggatctggacggtgatggttacctggaatgggtggtcaccgcgccgggcgtcgcgggctgctatgcgcaagcgcctgaacaatgctggcagcgcttcacgccgctgtctgcgctgccagtggaataccgccactcgcgaatggagataaccgacgtcaccggtgcgggtcttgcggatatgctgctgatcggcccgaaaagcgtacgcctgtacagcggcagcggcagaggctggaaaaaagcacgaacggtcatgcaggacagtggcatcaccctgccggttcccggtacaaatgcccgtgtcatggtggcattcagcgatatggccggcagcggtcagcaacacctgacggaaatcaaagccagcggcgtacgttactggcccagcctggggcatggtcgctttgcggctccggtgacactccccggcttcagtcagcccgctgaaaccttcaacccggcacagctctatctggccgacgttgacggttccggcaccaccgatctgatctatgctctgagcgatcatctgctggtatggctaaaccagagcggaaacagctttgacgcgcctttccgtatcagtcttccagaaggcgtgcgctatgacaatacctgcagtttgcaggtcgccgatattcaggggctgggcatctccagcctggtgctatcggtgccacatccgacgccgcgccattgggtatgtcacctgacgacggaaaagccctggctgctcgacggcatgaacaacaatatgggtgcccgccatactctctgttaccgtagttcggcgcagttctggctggatgaaaaggctgctgctaccgccgatcgacccgcgccggcgtgttatctgccgtttgcgctgcatacactgagccgtactgaagtcagtgatgaaatcaccggaaaccggctcaccaggacgatacgctaccggcacggggtctgggacaggcgcgagcgagagttccgcggctttggctttgttgaagtcagcgatgccgaagcgctggcaaaacaaactgaggggatgagcgcaccagcagttaaacgcagctggtatgctaccggactggcagccgtggatgcacagctcccggatgagttctggaaaggggatcatgcagcctttgccggttttacccctcgctttaccaccggcgatggcgaacaagaggcggcactggataccatcagcgacgatacccgtttctggctgacccgggcgattcgcggtacgctgctgcgtagcgaactgtatggcgcggatggcagcagccaggccgggatcccttacagcatcacggaatcgcggccacaagtgcggttgattactgaggcgggtaattcgccggtggtctggccctccgttatcgagaaccgcgccagtcattatgagcgcgtcagcagcgatccgcagtgcggccagcagatcctgttaaccagtaatgaatacggccagccgctccgtcagatcggcatcagttatccccggcgcaccaggcccgataccagcccctacccggacgatctgccggacggactgtttgccgacagctttgatgagcaacagcaggcgctgcgcctgacgctgacacaaagcagctggcatacgctgaaagatatcagcagcggcatctggctgccggccgtggcggatgcaacccgaagcgatctgttcgttcaccaggcagcgcaggtgccgccagcgggtcttacgctggagaatttactcaccgatagcgcgctgctgaccagcccggtttttggcggacagtcgcaaatctggtatcaggacagggcgggtcaggcgagcatcacctcacccgattttcccccccgaccgtcctttagcgaaaccgcagcgctggacgaggcacaggtcagcgcgctgtcagccgatattgatcaaacgaagctggagcaggcgggctatacccgctcagcgtatctgtttgcacgcagcggtgaggagagtaaaacgctgtgggcagtgcgccagggatatatcaccttcagcggcgcagaccatttctatctgccgattgccgcacagcagacgctgctggccggtaaaaccacagtcacctatgatccgtacgactgtgttgtcttacaggcaaaggacgccgcaggtgcggttacctccgcgacatacgactggcgttttctcgcgccgacgcagattactgatattaacgataatctgaaaagcgtcacgctggatgcgctgggtcgggtaacgtcgcagcgtttcagcggcactgaaaacggaaagccggcgggctacagcgatgacgagtttccactgccggccagcgccgatgcagcgctggcgctcagtgccccgctaccggtggcacagtgcatcatctacgtaccggacagctggatgctgaccggggagcagcagcagccgccgcacgtgataacgctgctcaccgaccgttacgacagcgacagtcagcagcagatccgtcagcaggttgttttcagcgatggttttggccgggtgctgcaggctgcctcaaggcaggtgaacggcgaagcgtggcagcgggcggcaaacggctcgttcgttgccggcacgaacgattcgcccgtgctgactgagacaacgttccgctgggccgttaccggacgcactgaatatgacaataagggacaggccatccgtgcttatcagccatattttctggacagctggaaatacgtgcgtgacgacagcgcgcgacaggatctgtacgccgacacccactattacgatccggtggggcgggagcggcaggtcattaccgcaaaaggctggctgcggcgcgtcactcacaccccctggttcgtagtcagcgaagacgaaaacgatacccaggcgtag

SEQ ID NO: 7 - DNA, 3015 bp, Pantoea agglomerans
  source 1..3015: mol_type = genomic DNA
atgtccgccgcgtatgtcttaagtaatttatcttataaactggagaatcctatgagcacctcgctttacagcaggaccccctcagtcacgatcctcgacaaccggggcctgaccgtacgcggtatcgcgtaccagcgccatccggataccccggcggtgaccagtgaacgcatcacccgccatcagtacgacgcccgcggctttctgatgcaaagcgccgacccgcgcctgcacgacgccgggctggcgaacgtcagctaccggaccaacctgaccggcagcgttctccgctcacagggcgtggataacggcatcaccgtgacgctgaacgatgccgccggacggccgtttctggcggtcagcaacatcagcactgccggtgatggcacggaggacagaagccaggcagtgacccgtacgtgtcagtacgaggacgccaccctgcccggacgtccgttaagtattacggagcaggtgaatggtggagccgcccgcatcacggagcgcttcgtctatgccggtaacgctgttgaggagaaagccctgaacctcgccgggcagcccgtcagccactatgataccgccggtctgacacagacagacagcatcgccctgaccggcgtgccgctctccgtcacccgccgcctgctgaaggacgcagacaatcctgacgccgtggctgactggcagggaacagacgcctccgtctggaacgacccgctcgacgtggaaacatacactaccctgtccacggcagacgccaccggcgcggtgctgaccaccaccgatgcgaagggaaacctgcagcggctggcctacgacgtggcgggcctgttgtcgggcagctggctgacgctgaaggatggcacggagcaggttatcgtgacgtccctgacctactccgccgccgggcagaagctgcgcgaggagcacggcaacggcgtggtgaccacctacacgtatgaagccgagacgcagcgcctgaccggcattaaaacggcgcggccggccggacacacctcaggtgcgaaggtgctgcaggacctgcgatacacctatgacccggtgggcaacgtcctgaaaatcagcaacgatgccgaagagacccgcttctggcgtaaccagaaagtggcgccggagagcgcgtacgtttatgacagcctgtaccagctggtcagcgccaccggacgcgagatggcgaacgccggtcagcagggcagcagctcatcgtcagccaccgtcccccttcccgccgacagttccgcgtttacaaactatacccgcaactatacttacgatgaggccggcaacctgacgcaggtccgtcataccccggctacgggcagcggctacaccacaaaaataaccgtctctgataaaagcaaccggggtgtgctgagcacgctgacggaaaatccctccgacgttgacgcgctgttcacggcgggcggccagcagaaacagctgcagccggggcagagtctcatctggacgccgcgtaacgagctgctgaaggtgacgccggtagcacgtgacggcggtgcggatgacagcgaaagctaccgctacgacgggggcagcctgcggctgctgaaggtcagcgtgcagaaaaccgggaacagcacgcagacgcagcgggcgctgtacctgccaggactggagctgcgcaacacaacatccggtgatacggaaacggagagcctgcaggtggttaccgtgggtgaagcggggcgcgcgcaggtgcgggtgctgcactgggagagcggaacgccggacagtgtcagcaacgacccggtgcgttacagctacgataacctgaccggcagcagcgggcttgagctggacagcagcggcaatattatcagcatggaggaatactatccgtacggcggcacggcggtctggacggcgcgcagcgcggtggaggcgaagtacaaaaccgtgcgctactcggccaaggagcgtgacgccacggggctgtactactacgggtaccggtactaccagccgtgggctggccgctggctgagcgcggacccggcgggcacggcggacgggctgaacctgttcaggatggtaagaaataatccggttacgcttaaggacacaaacgggttgatcagtacgggtcaggatgcccggaaattagtggccgaagcatttgttcaccctttgcatatgactgtctttgaaagaatttcttcagaagaaaatcttgcaatgagcgtgagagaggctggcatttatactatttcggcactgggtgaaggtgctgcagcaaaagggcataatattcttgagaagaccattaaacctggttcattaaaggctgtttatggtgataacgccgaatccattcttgcgcaggcaaaacgcagcggttttgttggccgggtaggtcagtgggatgcatccggtgtacggggaatttatgcacacaacacaccaggtggcgaagacctggcctatccagtcaacttaaaaaatagttctgctaatgaacttgttaatgcatggataaaatttaaaatcatcacgccttataccggtgattatgacatgcacgatattattaaaatctcggatggaaaagggcatgtgcccatggcggaaagtaatgaggaaaaaggtgtaaaggatatgattaatgaaggtgttgcgcaggtcgaccctgccagaccctttacgtctacagcgatgaatgttgttcgccatggccctcaggtaaactttgttccctatatgtgggaacatgagcacgaaaatgtcgtaagggataatggttatctgggagtggtagctcgtccgggtccattccctgttgcgatggtacataagggtgaatggactgttttcgacaataaaaacgagctgtttgagttttataaatctacaaacactcctcttcccgaacactggtctcaggattttgttgagagagggaaaggaaatgttgcaacgccccgacacgctgaaattcttgatcgtaattcctcgcgtctaagagcggcctga

SEQ ID NO: 8 - DNA, 2916 bp, Pantoea agglomerans
  source 1..2916: mol_type = genomic DNA
atgtgtagcgttgccgattttgatcggctgcacaacataaaacaggagaatatcatgggcacctcgctttacagcaagaccccctcagtcacgatcctcgacaaccgcggcctgtccgtacgcgatatcgcgtaccagcgccatccggataccccggcggtgaccagtgaatgcatcacccgccatcagtacgacgcccgcggctttctgatgcaaagcgccgacccgcgcctgcacgacgccggcctggcgaacttcagctaccggaccgacctgaccggcagcgttctccgctcacagggcgtggataacggcatcaccgtgacgctgaacgatgccgccggacggccgtttctggcggtcagcaacatcagcactgccggtgatggcacggaggacagaagccaggcagtgacccgtacgtgtcagtacgaggacgccaccctgcccggacgtccgttaagtattacggagcaggtgaatggtggagccgcccgcatcacggagcgcttcatctatgccggtaacgctgttgaggagaaagccctgaacctcgccgggcagcccgtcagccactatgataccgccggcctgacacagacagacagcatcgccctgaccggtgcgccgctctccgtcacccgccgcctgctgaaggacgcggacaatcctgacgccgtggctgactggcagggaacagacgcctccgtctggaacgacccgctcgacgtggaaacatacactaccctgtccacggcagacgccaccggcgcggtgctgaccaccaccgatgcgaagggaaacctgcagcggctggcctacgacgtggcgggcctgttgtcgggcagctggctgacgctgaaggatggcacggagcaggttatcgtgacgtccctgacctactccgccgccgggcagaagctgcgcgaggagcacggcaacggcgtggtgaccacctacacgtatgaagccgagacgcagcgcctgaccggcattaaaacggcgcggccggccggacacacctcaggtgcgaaggtgctgcaggacctgcgctacacctatgacccggtgggcaacgtcctgaaaatcagcaacgatgccgaagagacccgcttctggcgtaaccagaaagtggtgccggagagcgcgtacgtttatgacagcctgtaccagctggtcagcgccaccggacgcgagatggcgaacgccggtcagcagggcagcagctcatcgtcagccaccgtcccccttcccgccgacagttccgcgtttacaaactatacccgcaactatacttacgatgaggccggcaacctgacgcaggtccgtcataccccggctacgggcagcggctacaccacaaaaataaccgtctctgataaaagcaaccggggtgtgctgagcacgctgacggaaaatccctccgacgttgacgcgctgttcacggcgggcggccagcagaaacagctgcagccggggcagagtcttatctggacgccgcgcaatgagctgctgaaggtgatgccgataatgcgtgacggcggtacggatgacagcgaaagctaccgctacgacgggggcagccagcggctgctgaaggtcagcgtgcagaaaaccgggaacagcacgcagacgcagcgggcgctgtacctgccggggctggagctgcgcaacacaacatccggtgatacggaaacggagagcctgcaggtggttaccgcgggtgaagcggggcgcgcgcaggtgcgggtgctgcactgggagagcggaacgccggacagtgtcagcaacgacccggtgcgttacagctacgataacctgaccggcagcagcgggcttgagctggacagcagcggcaatattatcagcatggaggaatactatccgtacggcggcacggcggtctggacggcgcgcagcgcggtggaggcgaagtacaaaaccgtgcgctactcggccaaggagcgtgacgccacggggctgtactactacgggtaccggtactaccagccgtgggctggccgctggctgagcgcggacccggcgggcacggtggacgggctgaacctgttcagaatggtgcgcaataatccgctcacattaaaggataacgatgggctaaaaccaataaatgaaaatttcagagaaaataaaggcgatctggtttatgggctggctgccccccgaggagcttatatatcaacggcaatagggcgcgaattcgctccagaagagaaagatgcacctgcttcaattattgacttatataacaatacggtttccggacaagcccttcttagcgttgatttcaaaatactgcaagacttcatgaagtcaccaaaaaaacatgaaaaaaaactagcccctccatctaacattaaagagttagtaaaaaaatccagggtttatcctctgtgggaggattattttctggcaggggaaaataaccccaaatttaacattgcatccatatataaagaggtcagaaaagatgcaggaaagacccagtatcatgagtggcatatagcgggaggacaatcggcacctaagctactttggaaacgaggaagtaaattgggtattgaaatggcggccagcggtgctggtaataaaatccattttgttcttgatgaactggatatatcaaatgttgtaaataaggaagggccggggggaaaatctataacggccagtgagttacgttatgcgtacagaaatcgtgaaagactgactggaaatatacatttttacaaaaataatgctgaaactggcgcgccatgggatacaaatgctgaactgtgggcatcttaccaccccaaaccaaagcataaaggaaatgaatcgacacacatgatgtctcaaaggagaaatggtagtctgttcaaatctatgagaaaggttttctcgagaaattaa

SEQ ID NO: 9 - DNA, 7557 bp, Pantoea agglomerans
  source 1..7557: mol_type = genomic DNA
atgtatctgaccgaagaaatacttgccaaactgaatgccggaaacggcaaactacaatctactgtagagcagataattacgctgccagatattatgctgcactcttttgctcaggtaaaagaactggcaggagacaagttaagttggggtgagaaaaacttcctttatcaacaggctcagaaacagctgaaagaaaataaaatggcggaatcccgcattctcagccgtgccaacccgcaactggcaaatgctgtccggatgggcatccgtcagtctgcgatgctgggtagctatgacgacctgttcccgcagcgcgccagccgctttgttaagccgggtgcggtggcctcaatgttttcaccggctggttatctgaccgagctgtatcgggaagcccgaggcctgcacgacgacacgtcagactatcatctggatacccgccgtccggacctggcatcaatggtgttgtctcagtcaaatatggacactgagttgtccaccctgtcgctctccaatgaactgttgctgaagttaattcagtcaaaggaaagcctgaattatgaccaggttattgaaaagctggcgacttacagactgaccggcaccacgccttacaatcaaccctatgaaaccatccgtcaggctattttgctgcaggacccggagtttaacgcattcagtaataatccggcagtggccgtaaaaatcaacaccagcgggctattaggtattacttccgatatcgccccggagctgcatgcgatactgactgaagagataacagaaaaaaaaacggaagcactgattaaaaagaacttcggcgatgccaatatcaaccagttccaaaatcttgcgtggctggcccactggtacggcttgtcctatgaggagcttaataacctggtaggcatgatttggtccagagatgatcttgaccccgctgttgagcactataaaaattccagcctggtcactttggtggctgaagacggtggatcgcttaacgcggtgttgattaagcgtactaaaggccatgattccgatgatatgcattatgcggaattaattcctgtgggaggagacaaatttcagtacaacttcagccttattgatgctgaaagcagtagtgtttattatcaattcggtacaaaaggaaagaactcccaagatttagttcctgtaatccatgagcctttgctgggtaatactccctatgctgttacattcacacttacacaagagcagctaagtaacccagttgaaatatccctgacgcatggtagtggcggtggtgatcgccttacctcaacaattttcactgttacgacttacccatttgataccttcctgctgaagctgaataaactcatacgcctctataaagccaccggtatctccccggccagcatcaggaccgtgattgaaagcgataacactgaccttatcatcacagaaagcgtattaaaccagctattctggactaattactatacacaaacgttcgaaatggaattttctgccgcactggtgctggcaggagcggacatcggtcagatagcacacagtgaaagccagccaagtgcgttcacccgcctgtttaacacaccgttgctggataaccagcagttttcggccagcgacgagtcactggatctggagccgggtaagggagccgatgctttccgtatcgctgtactcaagcgtgcattgcaggtgaatgacgccggactgtataccctttatggtctgagtttcaccgataaagataaaaacggtaagttgattccgttcaccaccaatattgagaacctttctgccctctatcgcacccgactgctggccgacatatttaatatttctgttactgagctgagcatgctgctgtcggtttcaccttatgccagtcagaaggtggacagccttaaaggtcaggcactatatcagtttgttgctaccctcagtgactatatgcaacggctgaaagcgatgaactggagcgtcagcgatctctacctgatgctgaccaacagctacagcacggtactgtcgccagaaattaaaaacctgatgactaccctgaaaaatggactcagcgagcaggattttaataacacggatgaaatcgctcagctgaatgcgacggcacctttaatcgccgcagcgatgcagcttgacttcacagaaaccgcagcagcactgctggaatggcttaatcaattgcaaccagcagggctgacagtggcaggtttcctgtctcttgtgaatcagacgacactcgaagataaggatgttgtaaaactggtctctttctgccaggttatggggcagcttgcactgatcgtgcgcaaggcggctctgggctccagcgaaatcacctttgcagttgcgcatccggctatttttaaaaaagatgcgaactcactggctcaggatattggcacgctctttgacctgacccagctgcatgcatttctgacagactgtggtacttatgcctctgaaattctcacctcactgaatgaagggaatctcgacgttagcacggtggcgacggcgctgacgctggacaaaacttcactggcgcaggcacttgctcaggtttcagaatctcaggccttttctaactggcacgaactgcgtgatgcacttcagtggacagatgccgccagcattttcaacatcacaccagtggctctgactgcgatggtgaacctgaaattcagcggtgacaactcttctccgtatcaggagtgggtaacggtcagcaaagctatgcaggtcgggctgaatcagacgcaaagcgctcagctgcaagcctcgctggatgaatccctcagcgcagcactcagcgcctacgtcattaagaacataacacccccatcagtaactgatcgcgacgaactttacggctggctgctgattgacaatcaggtctctgcacagattaaaactacccgcattgctgaagcgattgccagcgttcagctttacgtaaaccggtcactgacgggtcaggaagatggcgtggatagcaaggttaaatccggccagttctttacggcagactgggatacttataacaaacgctacagtacatgggccggtgtgtcggagctggtctattatccggaaaactatgttgatccgacgctgcgtatcgggcagaccgggatgatggatgaaatgttgcagacgctcagccagagccagattaatttagacaccgtcagtgatggtatggggcgttaccttactgattttgaagaaatagcaaatctaaaattcctcagtggttatcatgataatgtttctggccgtcaggggaaaacctggtttatcggtggcagtcagtctgaaccccaaaaattttactggcgatccctggattacagtaaaggcgatggggaggaattcgctgccaatgcatggtcagaatggaaccatatctcatgtgcaataacacccttacctggttttgttcgtgtggttttatttaactcccgactatatcttgcttgcgtggaaaaaaaagaaattcgggatagtgaaaacaaaaataaagcatcgtatcaattaaagatagctcacatcctttacaatggtgagtggagcgctcccttctcacacgatattactgatttatatgaggcaggctttgatccgagtacaacagtaatgcacttatctgtacatgatgagagtgatgcaatagtttgtatatttaataacagcgcgctagaaagtgacaaaaataaaggggtggcagtcaatgctgatatgtcatttaacaacattgacagcaaaagagtagatcagataattagtcttttagttcctgatcgttttatagatgaaggtaatgttatagataatttagtttctgagttaaagggatcggaagtcacggaaaataaaaaaacgctggagaatgattcgttcactatagatggatcaataaatttgaataagcattctatcgatatcacagggaaggccaatttagatattcaggcatcaattgctgtgcgtagtaaagcatctcctactagccatgagcgcgagctaataggctggttagatgaatctcaatttgattacattcgattattcaggggtggctataattttggccagaacgacggcattttggaatcatgcatgatttcggcagttaatagtgcctatacctgcttccttttacgagctgaccacttcagtggtttatttagttatggatatgacctttttgtattcaacggtgacgggtcaaaaacatatacacctcaagtgttgtttgaagatgatattcaagggactatggtgctcaagatcgtgctcctaaatgaggataaaaattcaaaactggaaaactttgaatccctggggcttatgaaaacatcagcaggcgatcatcagggagaaatagtttgcgaacttgctaaaagaaggacacctgagccttactgtgtagaattgagtcgctacttaccctcgaatgttactgttaccgttacatcaccatcggggaactttactgccaaagactatgtgttacctcttcccgcattcaataatggcgacgctgactataaattcgcaccattccccctctcgcttgaaagtatatggggagatggaaaaagtaccagtcgggacattaagtttacaataagcgtaaaagatacttgcggcaaggtggccacctcagagctaatctttacactttataaaaacacctcgcctgaattaattacactgaaaacgagtgacgcgggagcgcagtatatgcagcagggagtgtaccgcacaaggcttaataccctgtttgcacagaaattgatcaagcgtgttagcgccggaattgatgcagtgctgtcgtgggaaacccagcagttgcaggagcctaaactgggtactggcagttacatttcagtgcttatccccgcctatatcaaacttgagcacggagatagcagacaggctaacctgcagtttagtaatgtcgatcaaacaggaccggataatgggaattatatattatggtccggctcattaaatgacactccgcagcaggtcacgatttttgtgcccacgatgcaaactattggcgagctgcaattcccttatgaccggactagtggcctgaatctgagtttagcatgtgcagctggagtttatttgcaggggacattcaagaatatatctgcgtccgatttatctttaactgagtttgttgctgcaaagaacaatgactctaaacgggatgtcgaagtgacagtattaacttcaatcaatacggagccaatggacttcaagggtgccaacgccctctatttctgggagatgttctactacctccctatgatggtgtttaaacgccttctcagcgaaagtcggtttactgaagccactcagtggataaggtacgtctggaacccggacggctacctggtaaacgacacgcccgccacctaccagtggaacgtgcgcccgctggaggatgaaacctcctggcacgctaacccgctggactccgtggacccggatgccatagcccaggctgacccgctgcactacaaggtcgccacctttatggcgtaccttgacctgctgattgcccgcggcgacgcggcctaccgtcagcttgagcgcgatgcgctcagcgaagcaaaaatgtggtacgtgcaggcgctggacacccttggcgatgagccgtacctgagccagaacacaggctgggcgtccccatgcctgacggatgctgccgatgagaccacccataaaaacaggcagcaggcaatgctgaccgtgcgccagaaggttgcctccagcgaactgcgcaccgccaactccctaaccgccctgttcctgccacagcagaacgcgaagctggcaggctactggcagacgctgaaccagcgcctgtataacctgcgcaacaacctctccattgacggtaacccgctgtcgctgtccatttatgccaccccgactgacccggcggcgctgctcagctcggcggtgattagttctcaggggggcagtgacctgccagcggccgttatgccgctgtaccgcttcccggtgattctggaaagcgcacggagcatggtgaatcagctgacccagttcggcagcacgctgctcggcatcaccgagcgtcaggatgcagaggcgctgtctgatctgctgcagacacagggggctggactggcgctgcaaagcattgccctgcagaacagtaccatcagcgagattgatgcggatagggccgcgctcagggagagcctcagtggcgcacagtcgcgcctcaacagctataccaccctgtatgatgaaaatgttaatgctggtgaaacgcacgccatgaacctgtttctttcctccgccatcctggcagatggcgggcaggcctatcataccgccgcgggtgcgcttgacctggcgccgaatatctttggcctggccgacgggggttcccgctggggtgcggcatttaccgcaatggccggaatagctgatttggccgcctcggccacccatacggccgccgaccgcatcagccagtctgaggcataccgccgccgccgccaggagtgggaaatccagcgcaacgcggcgcagttcgaggtcagccaaatcaatgcccagctggacgcgctggcggtgcgtcgtgaaagcgccgtgctgcagaagacctatctggaaacacagcagggccagatgcaggcgcagatgaccttcctgcagaacaagttcaccagcaaagcactgtacaactggctgcgcggtaaactggcggccatctactatcagttctatgacctgaccgtttcacgctgtctgatggcagaagctgcctacagctgggagatgaaaggctctcaggatacgggcacctttatccgtcccggcgcctggcagggaacctatgccggcctgatggcaggggaaacgctgatgctgaatctggcacagatggaaaacagctatctgacaaaagaggagcgccagaaagaggtcacgcgcacggtctgcctgtctgaagtttatgcagggctctcttcgggttcgttcgcgctggctgataccgtcaccacactggtgggtagcgggaaaggcaccgccggcacgaacgataacggagtgaagatcgatggcaagcagcttctggctaccctgaaactctccgatctgaacattaagacggattatccagagtcactggacaaagccaaacgcattaagcaaatcagcgtgacgctgccgacgctggtcgggccgtatcaggacgtccgggcggtactgagctatggcggcagcgtggttctgccacgcggctgcacggcggtcgccgtttcgcacggcatgaacgacagcggccagttccagctggacttcaatgacagccgctggctgccttttgaaggtatacctgttgatgattccggtacgctgacgctcagcttcccggacattaccgataagcaacaggaaaatctgctgctcagtctgagcgacatcatcctgcacatccgctataccatcgcaagctga

SEQ ID NO: 10 - DNA, 4266 bp, Pantoea agglomerans
  source 1..4266: mol_type = genomic DNA
atgcaaaatacagatcagatgagcctgacgcccccttccttaccctcaggtgggggtgccgtcaccggactgaaaggcgatatgtcaggagccggacccgatggcgccgccacgctgagccttcccttgccgatcagccccggacgtggctatgccccgtcgctgtcactgggttaccacagtcgtaacggcaacggtgtttttggcgcaggctggagctgcggtcagatggctattcgcctccaaacccgcaaaggcgtgccgttttatgacggcagcgacgtctttaccgctcctgatggtgaggttctggtgccggcgctggacgccagcggcaaggctgaggttcgcacgaccactacgctgctcggcgaaaacctcggcggcacctttaccgtacagacctaccgttcccgagtggaaaccgacttcagtcgcctggagcgctgggttccgcagaccgacgcagcggctgatttctggttgatttatagtccggacggccagatccacctgctgggtcgtaacccgcaggcgcgggtgaacaaccctgaggatacaacccagaccgccgcctggctgatagagtcgtcggtctccgccagcggcgagcagatttactggcaataccggcaggaagatgagctgggctgtacgcaggatgagaaaacggctcacgcacacgcgctcgcccagcgctatctggtggcggtatggtatggcaataaagcggccagccggacgctgccggggctgctgtctgttcctgcggctggcagctggctgtttacgctggcgctggactacggtgagcgggcgacagatcctgcaacaccaccggcctggctgtcaccgggcagtggcacatggctctgccggcaggatgtgttctccagctgggaatatggctttgagctgcgcacgcgtcgcctgtgccgacaggtactgatgtatcatgacgtcgcggcgctggcaggtcagtcaggttcagatgccgtgccacagctggtcaccagactgctgctggactataacacgtctccgtcgctgactaccctgaaaaccgcacagcaggccgcctgggaaccggatgggacgttgcgcagtctgccgccgctggcgttcagctggcagaccttcccgtcaacaccagagaaaagtgtcagctggcagcggcggaatgacatggggaaactcaacccacagcagccttatcagatggttgacctgcacggtgaaggactggcgggtatcctctatcaggacagtggtgcctggtggtatcgggagccggttcgtcagtcgggtgatgatgataatgccgtgacctgggcggctgcccgaccgctgccggcgttccctgctctgcgcaagggcggaatgctgctggatctggacggtgatggttacctggaatgggtggtcaccgcgccgggcgtcgcgggctgctatgcgcaagcgcctgaacaatactggcagcgcttcacgccgctgtctgcgctgccagtggaataccgccactcgcgaatggagatagccgacgtcaccggtgcgggtcttgcggatatgctgctgatcggcccgaaaagcgtacgcctgtacagcggcagcggcagaggctggaaaaaagcacgaacggtcatgcaggacagtggcatcaccctgccggttcccggtacaaatgcccgtgtcatggtggcattcagcgatatggccggcagcggtcagcaacacctgacggaaatcaaagccagcggcgtacgttactggcccagccttgggcatggtcgctttgcggctccggtgacactccccggcttcagtcagcccgctgaaaccttcaacccggcacagctctatctggccgacgttgacggttccggcaccaccgatctgatctatgctctgagcgatcatctgctggtatggctaaaccagagcggaaacagctttgacgcgcctttccgtatcagtcttccagaaggcgtgcgctatgacaatacctgcagtttgcaggtcgccgatattcaggggctgggcatctccagcctggtgctatcggtgccacatccgacgccgcgccattgggtatgtcacctgacgacggaaaagccctggctgctcgacggcatgaacaacaatatgggtgcccgccatactctctgttaccgtagttcggcgcagttctggctggatgaaaaggctgctgctaccgccgatcgacccgcgccggcgtgttatctgccgtttgcgctgcatacactgagccgtactgaagtcagtgatgaaatcaccggaaaccggctcaccaggacgatacgctaccggcacggggtctgggacaggcgcgagcgagagttccgcggctttggctttgttgaagtcagcgatgccgaagcgctggcaaaacaaactgaggggatgagcgcaccagcagttaaacgcagctggtatgctaccggactggcagccgtggatgcacagctcccggatgagttctggaaaggggatcatgcagcctttgccggttttacccctcgctttaccaccggcgatggcgaacaagaggcggcactggataccatcagcgacgatacccgtttctggctgacccgggcgattcgcggtacgctgctgcgtagcgaactgtatggcgcggatggcagcagccaggccgggatcccttacagcatcacggaatcgcggccacaagtgcggttgattactgaggcgggtaattcgccggtggtctggccctccgttatcgagaaccgcgccagtcattatgagcgcgtcagcagcgatccgcagtgcggccagcagatcctgttaaccagtaatgaatacggccagccgctccgtcagatcggcatcagttatccccggcgcaccaggcccgatgccagcccctacccggacgatctgccggacggactgtttgccgacagctttgatgagcaacagcaggcgctgcgcctgacgctgacacaaagcagctggcatacgctgaaagatatcagcagcggcatctggctgccggccgtggcggatgcaacccgaagcgatctgttcgttcaccaggcagcgcaggtgccgccagcgggtcttacgctggagaatttactcaccgatagcgcgctgctgaccagcccggtttttggcggacagtcgcaaatctggtatcaggacagggcgggtcaggcgagcatcacctcacccgattttcccccccgaccgtcctttagcgaaaccgcagcgctggacgaggcacaggtcagcacgctgtcagccgatattgatcaaacgaagctggagcaggcgggctatacccgctcagcgtatctgtttgcacgcagcggtgaggagagtaaaacgctgtgggcagtgcgccagggatatatcaccttcagcggcgcagaccatttctatctgccgattgccgcacagcagacgctgctggccggtaaaaccacagtcacctatgatccgtacgactgtgttgtcttacaggcaaaggacgccgcaggtgcggttacctccgcgacatacgactggcgttttctcgcgccgacgcagattactgatattaacgataatctgaaaagcgtcacgctggatgcgctgggtcgggtaacgtcgcagcgtttcagcggcactgaaaacggaaaaccggcgggctacagcgatcacgagtttccactgccggccagcgccgatgcagcgctggcgctcagtgccccgctaccggtggcacagtgcatcatctacgtaccggacagctggatgctgaccggggagcagcagcagccgccgcacgtggtaacgctgctcaccgaccgttacgacagcgacagtcagcagcagatccgtcagcaggttgttttcagcgatggttttggccgggtgctgcaggctgcctcaaggcaggtgaacggcgaagcgtggcagcgggcggcaaacggctcgttcgttgccggcacgaacgattcgcccgtgctgactgagacaacgttccgctgggccgttaccggacgcactgaatatgacaataagggacaggccatccgtgcttatcagccatattttctggacagctggaaatacgtgcgtgacgacagcgcgcgacaggatctgtacgccgacacccactattacgatccggtggggcgggagcggcaggtcattaccgcaaaaggctggctgcggcgcgtcactcacaccccctggttcgtagtcagcgaagacgaaaacgatacccaggcgtag

SEQ ID NO: 11 - DNA, 2856 bp, Pantoea agglomerans
  source 1..2856: mol_type = genomic DNA
atgtccgccgcgtatgtcttaagtaatttatcttatcaacgggagaatactatgagcacctcgctttacagcaggaccccctcagtcacggtcctcgacaaccgcggcctgaccgtacgcgatatcgcgtaccaccgccatccggataccccggcggtgaccagtgaacgcatcacccgccatcagtacgatgcccgcggctttctgacgcagagcgccgacccgcgcctgcacgacgccgggctggcgaacttcagctaccggaccgacctgaccggcagcgttctccgcctacagggcgtcgataacggcatcaccgtggcgctgaacgatgccgccggacggccgtttctggcggtcagcaacatccgcacggccggtgatggctcggaggacagaagccaggcagtgacccgtacgtgtcagtacgaggacgccaccctgcccggacgtccgttaagtattacggagcaggtaaagggtggagccgcccgcatcacggagcgcttcatctatgccggtaacgctgttgaggagaaagccctgaacctcgccgggcagcccgtcagccactatgataccgccggtctgacacagacagacagcatcgccctgaccggcgtgccgctctccgtcacccgccgcctgctgaaggacgcagacaatcctgacgccgtggctgactggcagggaacagacgcctccgtctggaacgacccgctcgacgtggaaacatacactaccctgtccacggcagacgccaccggcgcggtgctgaccaccaccgatgcgaagggaaacctgcagcggctggcctacgacgtggcgggcctgttgtcgggcagctggctgacgctgaaggatggcacggagcaggttatcgtgacgtccctgacctactccgccgccgggcagaagctgcgcgaggagcacggcaacggcgtggtgaccacctacacgtatgaagccgagacgcagcgcctgaccggcattaaaacggcgcggccggccggacacacctcaggtgcgaaggtgctgcaggacctgcgctacacctatgacccggtgggcaacgtcctgaaaatcagcaacgatgccgaagagacccgcttctggcgtaaccagaaagtggcgccggagagcgcgtacgtttatgacagcctgtaccagctggtcagcgccaccggacgcgagatggcgaacgccggtcagcagggcagcagctcatcgtcagccaccgtcccccttcccgccgacagttccgcgtttacaaactatacccgcaactatacttacgatgaggccggcaacctgacgcaggtccgtcataccccggctacgggcagcggctacaccacaaaaataaccgtctctgataaaagcaaccggggtgtgctgagcacgctgacggaaaatccctccgacgttgacgcgctgttcacggcgggcggccagcagaaacagctgcagccggggcagagtctcatctggacgccgcgtaacgagctgctgaaggtgacgccggtagcacgtgacggcggtgcggatgacagcgaaagctaccgctacgacgggggcagcctgcggctgctgaaggtcagcgtgcagaaaaccgggaacagcacgcagacgcagcgggcgctgtacctgccagggctggagctgcgcaacacaacatccggtgatacggaaacggagagcctgcaggtggttaccgtgggtgaagcggggcgcgcgcaggtgcgggtgctgcactgggagagcggaacgccggacagtgtcagcaacgacccggtgcgttacagctacgataacctgaccggcagcagcgggcttgagctggacagcagcggcaatattatcagcatggaggaatactatccgtacggcggcacggcggtctggacggcgcgcagcgcggtggaggcggagtacaaaaccgtgcgctactcgggcaaggagcgtgacgccacggggctgtactactacgggtaccggtactaccagccgtgggctggccgctggctgagcgcggacccggcgggcacggtggacgggctgaatttgttcaggatggtaaggaataacccggtaacattggttgatgataatggtttattcacgtcctcccctttattggggatttatgaaaaggagatgaaaacctttgatagtatcaaattgtcgattggttcttataaatacaaaccatctaaatttgatgaaaagaaaggtaagtatgttagctcagataaatacaaactgataatggcagatgataacgatcttaatgggtatttatttgacgagcgcgagatgacaagccatctaaaggactatgctgataagttcagtaaaataagcaggctaaatataggcgatgagcggatgaaaaccaatattaattttgggactagaatatcaagatatttgctatcttcagcacaagcatcatcacgcgaaaatcgtgaagtagatgttttgtcattcgaaagaaaattttttgctgtagtaaagaaaaaagataaaagtcattattttggacgaaaaatatatgccataggagaagctcatgtactaacagattttgaagagaaaaaaagaaccattgccattaagactctagttgcgcacccctatacgcaaattaatgaaagcattaaaaatagaattaatgattttgataaagaatataacgttaaagggattggaacttttgcaacgtttaaagctacgaacaagctcataggtggtattaagggagctttaaaatataagactaaagtgttgactcaagcggtaaatgtacgctcggcagctatagcaataaagtatggggcaaagcacgttccgtaa

SEQ ID NO: 12 - DNA, 7572 bp, Pantoea agglomerans; identical to SEQ ID NO: 5
  source 1..7572: mol_type = genomic DNA

SEQ ID NO: 13 - DNA, 4266 bp, Pantoea agglomerans
  source 1..4266: mol_type = genomic DNA
atgcaaaatacagatcagatgagcctgacgcccccttccttaccctcaggtgggggtgccgtcaccggactgaaaggcgatatgtcaggagccggacccgatggcgccgccacgctgagccttcccctgccgatcagccccggacgtggctatgccccgtcgctgtcactgggttaccacagtcgtaacggcaacggtgtttttggcgcaggctggagctgcggtcagatggctattcgcctccaaacccgcaaaggcgtgccgttttatgacggcagcgacgtctttaccgctcctgatggtgaggttctggtgccggcgctggacgccagcggcaaggctgaggttcgcacgaccactacgctgctcggcgaaaacctcggcggcacctttaccgtacagacctaccgttcccgagtggaaaccgacttcagtcgcctggagcgctgggttccgcagaccgacgcagcggctgatttctggttgatttatagtccggacggccagatccacctgctgggtcgtaacccgcaggcgcgggtgaacaaccctgaggatacaacccagaccgccgcctggctgatagagtcgtcggtctccgccagcggcgagcagatttactggcaataccggcaggaagatgagctgggctgtacgcaggatgagaaaacggctcacgcacacgcgctcgcccagcgctatctggtggcggtatggtatggcaataaagcggccagccggacgctgccggggctgctgtctgttcctgcggctggcagctggctgttttcgctggtgctggactacggtgagcggacgacagatcctgcaacgctaccggcctggctgtcaccgggcagtggcacatggctctgccggcaggatgtgttctccagctgggaatatggctttgagctgcgcacgcgtcgcctgtgccgacaggtactgatgtatcatgacgtcgcagcgctggcaggtcagtcaggttcagatgccgtgccacagctggtcaccagactgctgctggactataacctatctccgtcgctgactaccctgaaaaccgcacagcaggccgcctgggaaccggatgggacgttgcgcagtctgccgccgctggcgttcagctggcagaccttcccgtcaacaccagagaaaagtgtcagctggcaacggcggaatgacatggggaaactcaacccacagcagccttatcagatggttgacctgcacggtgaaggactggcgggtatcctctatcaggacagtggtgcctggtggtatcgggagccggttcgtcagtcgggtgatgatgataatgccgtgacctgggcggctgcccgaccgctgccggcgttccctgctctgcgcaagggcggaatgctgctggatctggacggtgatggttacctggaatgggtggtcaccgcgccgggcgtcgcgggctgctatgcgcaagcgcctgaacaatgctggcagcgcttcacgccgctgtctgcgctgccagtggaataccgccactcgcgaatggagatagccgacgtcaccggtgcgggtcttgcggatatgctgctgatcggcccgaaaagcgtacgcctgtacagcggcagcggcagaggctggaaaaaagcacgaacggtcatgcaggacagtggcatcaccctgccggttcccggtacaaatgcccgtgtcatggtggcattcagcgatatggccggcagcggtcagcaacacctgacggaaatcaaagccagcggcgtacgttactggcccagcctggggcatggtcgctttgcggctccggtgacactccccggcttcagtcagcccgctgaaaccttcaacccggcacagctctatctggccgacgttgacggttccggcaccaccgatctgatctatgctctgagcgatcatctgctggtatggctaaaccagagcggaaacagctttgacgcgcctttccgtatcagtcttccagaaggcgtgcgctatgacaatacctgcagtttgcaggtcgccgatattcaggggctgggcatctccagcctggtgctatcggtgccacatccgacgccgcgccattgggtatgtcacctgacgacggaaaagccctggctgctcgacggcatgaacaacaatatgggtgcccgccatactctctgttaccgtagttcggcgcagttctggctggatgaaaaggctgctgctaccgccgatcggcccgcgccggcgtgttatctgccgtttgcgctgcatacactgagccgtactgaagtcagtgatgaaatcaccggaaaccggctcaccaggacgatacgctaccggcacggcgtctgggacaggcgcgagcgagagttccgcggctttggctttgttgaagtcagcgatgccgaagcgctggcaaaacaaactgaggggatgagcgcaccagcagttaaacgcagctggtatgctaccggactggcagccgtggatgcacagctcccggatgagttctggaaaggggatcatgcagcctttgccggttttacccctcgctttaccaccggcgatggcgaacaagaggcggtactggataccatcagcgacgatacccgtttctggctgacccgggcgattcgcggtacgctgctgcgtagcgaactgtatggcgcggatggcagcagccaggccgggatcccttacagcatcacggaatcgcggccacaagtgcggttgattactgaggcgggtaattcgccggtggtctggccctccgttatcgagaaccgtaccagtcattatgagcgcgtcagcagcgatccgcagtgcggccagcagatcctgttaaccagtaatgaatacggccagccgctccgtcagatcggcatcagttatccccggcgcaccaggcccgatgccagcccctacccggacgatctgccggacggactgtttgccgacagctttgatgagcaacagcaggcgctgcgcctgacgctgacacaaagcagctggcatacgctgaaagatatcagcagcggcatctggctgccggccgtggcggatgcaacccgaagcgatctgttcgttcaccaggcagcgcaggtgccgccagcgggtcttacgctggagaatttactcaccgatagcgcgctgctgaccagcccggtttttggcggacagtcgcaaatctggtatcaggacagggcgggtcaggcgagcatcacctcacccgattttcccccccgaccgtcctttagcgaaaccgcagcgctggacgaggcacaggtcagcgcgctgtcagccgatattgatcaaacgaagctggagcaggcgggctatacccgctcagcgtatctgtttgcacgcagcggtgaggagagtaaaacgctgtgggcagtgcgccagggatatatcaccttcagcggcgcagaccatttctatctgccgattgccgcacagcagacgctgctggccggtaaaaccacagtcacctatgatccgtacgactgtgttgtcttacaggcaaaggacgccgcaggtgcggttacctccgcgacatacgactggcgttttctcgcgccgacgcagattactgatattaacgataatctgaaaagcgtcacgctggatgcgctgggtcgggtaacgtcgcagcgtttcagcggcactgaaaacggaaagccggcgggctacagcgatgacgagtttccactgccggccagcgccgatgcagcgctggcgctcagtgccccgctaccggtggcacagtgcatcatctacgtaccggacagctggatgctgaccggggagcagcagcagccgccgcacgtgataacgctgctcaccgaccgttacgacagcgacagtcagcagcagatccgtcagcaggttgttttcagcgatggttttggccgggtgctgcaggctgcctcaaggcaggtgaacggcgaagcgtggcagcgggcggcaaacggctcgttcgttgccggcacgaacgattcgcccgtgctgactgagacaacgttccgctgggccgttaccggacgcactgaatatgacaataagggacaggccatccgtgcttatcagccatattttctggacagctggaaatacgtgcgtgacgacagcgcgcgacaggatctgtacgccgacacccactattacgatccggtggggcgggagcggcaggtcattaccgcaaaaggctggctgcggcgcgtcattcacaccccctggttcgtagtcagcgaagacgaaaacgatacccaggcgtag

SEQ ID NO: 14 - DNA, 3015 bp, Pantoea agglomerans
  source 1..3015: mol_type = genomic DNA
atgtccgccgcgtatgtcttaagtaatttatcttataaactggagaatcctatgagcacctcgctttacagcaggaccccctcggtcacgatcctcgacaaccggggcctgaccgtacgcggtatcgcgtaccagcgccatccggataccccggcggtgaccagtgaacgcatcacccgccatcagtacgatgcccgcggctttctgatgcaaagcgccgacccgcgcctgcacgacgccgggctggcgaacgtcagctaccggaccaacctgaccggcagcgttctccgctcacagggcgtggataacggcatcaccgtgacgctgaacgatgccgccggacggccgtttctggcggtcagcaacatcagcactgccggtgatggcacggaggacagaagccaggcagtgacccgtacgtgtcagtacgaggacgccaccctgcccggacgtccgttaagtattacggagcaggtgaatggtggagccgcccgcatcacggagcgcttcgtctatgccggtaacgctgttgaggagaaagccctgaacctcgccgggcagcccgtcagccactatgataccgccggtctgacacagacagacagcatcgccctgaccggcgtgccgctctccgtcacccgccgcctgctgaaggacgcagacaatcctgacgccgtggctgactggcagggaacagacgcctccgtctggaacgacccgctcgacgtggaaacatacactaccctgtccacggcagacgccaccggcgcggtgctgaccaccaccgatgcgaagggaaacctgcagcggctggcctacgacgtggcgggcctgttgtcgggcagctggctgacgctgaaggatggcacggagcaggttatcgtgacgtccctgacctactccgccgccgggcagaagctgcgcgaggagcacggcaacggcgtggtgaccacctacacgtatgaagccgagacgcagcgcctgaccggcattaaaacggcgcggccggccggacacacctcaggtgcgaaggtgctgcaggacctgcgctacacctatgacccggtgggcaacgtcctgaaaatcagcaacgatgccgaagagacccgcttctggcgtaaccagaaagtggcgccggagagcgcgtacgtttatgacagcctgtaccagctggtcagcgccaccggacgcgagatggcgaacgccggtcagcagggcagcagctcatcgtcagccaccgtcccccttcccgccgacagttccgcgtttacaaactatacccgcacttatgcttacgatgaggccggcaacctgacgcaggtccgtcataccccggctacgggcagcggctacaccacaaaaataaccgtctctgataaaagcaaccgggccgtgctgagcgtgctgacgaaaaatccctctgatgtggacgcgctgttcacggcgggcggccagcagaaacagctgcagccggggcagagtcttatctggacgccgcgcaatgagctgctgaaggtgatgccgataatgcgtgacggcggtacggatgacagcgaaagctaccgctacgacgggggcagccagcggctgctgaaggtcagcgtgcagaaaaccggcaacagcacgcagacgcagcgggcgctgtacctgccaggactggagctgcgcaacacaacatccggtgatacggaaacggagagcctgcaggtggttaccgcgggtgaagcggggcgcgcgcaggtgcgggtgctgcactgggagagcggaacgccggacagtgtcagcaacgaccagctacgctacagctacgataacctgaccggcagcagcgggcttgagctggacagcagcggcaatattatcagcatggaggaatactatccgtacggcggcacggcggtctggacggcgcgcagcgcggtggaggcgaagtacaaaaccgtgcgctactcgggcaaggagcgtgacgccacggggctgtactactacgggtaccggtactaccagccgtgggctggccgctggctgagcgcggacccggcgggcacggcggacgggctgaacctgttcaggatggtaagaaataatccggttacgcttaaggacacaaacgggttgatcagtacgggtcaggatgcccggaaattagtggccgaagcatttgttcaccctttgcatatgactgtctttgaaagaatttcttcagaagaaaatcttgcaatgagcgtgagagaggctggcatttatactatttcggcactgggtgaaggtgctgcagcaaaagggcataatattcttgagaagaccattaaacctggttcattaaaggctgtttatggtgataacgccgaatccattcttgcgcaggcaaaacgcagcggttttgttggccgggtaggtcagtgggatgcatccggtgtacggggaatttatgcacacaacacaccaggtggcgaagacctggcctatccagtcaacttaaaaaatagttctgctaatgaacttgttaatgcatggataaaatttaaaatcatcacgccttataccggtgattatgacatgcacgatattattaaaatctcggatggaaaagggcatgtgcccatggcggaaagtaatgaggaaaaaggtgtaaaggatatgattaatgaaggtgttgcgcaggtcgaccctgccagaccctttacgtctacagcgatgaatgttgttcgccatggccctcaggtaaactttgttccctatatgtgggaacatgagcacgaaaatgtcgtaagggataatggttatctgggagtggtagctcgtccgggtccattccctgttgcgatggtacataagggtgaatggactgttttcgacaataaaaacgagctgtttgagttttataaatctacaaacactcctcttcccgaacactggtctcaggattttgttgagagagggaaaggaaatgttgcaacgccccgacacgctgaaattcttgatcgtaattcctcgcgtctaagagcggcctga

SEQ ID NO: 15 - DNA, 2079 bp, Pantoea agglomerans
  source 1..2079: mol_type = genomic DNA
atgtgtagcgttgccgattttgatcggctgcacaacataaaacaggagaatatcatgggcacctcgctttacagcaagaccccctcggtcacgatcctcgacaaccgcggcctgaccgtacgcgatatcgcgtaccagcgccatccggataccccggcggtgaccagtgaacgcatcacccgccatcagtacgatgcccgcggctttctgatgcaaagcgccgacccgcgcctgcacgacgccgggctggcgaacgtcagctaccggaccaacctgaccggcagcgttctccgctcacagggcgtggataacggcatcaccgtgacgctgaacgatgccgccggacggccgtttctggcggtcagcaacatcagcactgccggtgatggcacggaggacagaagccaggcagtgacccgtacgtgtcagtacgaggacgccaccctgcccggacgtccgttaagtattacggagcaggtgaatggtggagccgcccgcatcacggagcgcttcgtctatgccggtaacgctgttgaggagaaagccctgaacctcgccgggcagcccgtcagccactatgataccgccggtctgacacagacagacagcatcgccctgaccggcgtgccgctctccgtcacccgccgcctgctgaaggacgcagacaatcctgacgccgtggctgactggcagggaacagacgcctccgtctggaacgacccgctcgacgtggaaacatacactaccctgtccacggcagacgccaccggcgcggtgctgaccaccaccgatgcgaagggaaacctgcagcggctggcctacgacgtggcgggcctgttgtcgggcagctggctgacgctgaaggatggcacggagcaggttatcgtgacgtccctgacctactccgccgccgggcagaagctgcgcgaggagcacggcaacggcgtggtgaccacctacacgtatgaagccgagacgcagcgcctgaccggcattaaaacggcgcggccggccggacacacctcaggtgcgaaggtgctgcaggacctgcgctacacctatgacccggtgggcaacgtcctgaaaatcagcaacgatgccgaagagacccgcttctggcgtaaccagaaagtggtgccggagagcgcgtacgtttatgacagcctgtaccagctggtcagcgccaccggacgcgagatggcgaacgccggtcagcagggcagcagctcatcgtcagccaccgtcccccttcccgccgacagttccgcgtttacaaactatacccgcaactatacttacgatgaggccggcaacctgacgcaggtccgtcataccccggctacgggcagcggctacaccacaaaaataaccgtctctgataaaagcaaccggggtgtgctgagcacgctgacggaaaatccctccgacgttgacgcgctgttcacggcgggcggccagcagaaacagctgcagccggggcagagtctcatctggacgccgcgtaacgagctgctgaaggtgacgccggtagcacgtgacggcggtgcggatgacagcgaaagctaccgctacgacgggggcagcctgcggctgctgaaggtcagcgtgcagaaaaccgggaacagcacgcagacgcagcgggcgctgtacctgccagggctggagctgcgcaacacaacatccggtgatacggaaacggagagcctgcaggtggttaccgtgggtgaagcggggcgcgcgcaggtgcgggtgctgcactgggagagcggaacgccggacagtgtcagcaacgacccggtgcgttacagctacgataacctgaccggcagcagcgggcttgagctggacagcagcggcaatattatcagcatggaggaatactatccgtacggcggcacggcggtctggacggcgcgcagcgcggtggaggcgaagtacaaaaccgtgcgctactcgggcaaggagcgtgacgccacggggctgtactactacgggtaccggtactaccagccgtgggctgagcgcggacccggcgggcacggtggacgggctgaacctgttcagaatggtgcgcaataa

SEQ ID NO: 16 - DNA, 7557 bp, Pantoea agglomerans
  source 1..7557: mol_type = genomic DNA
atgtatctgaccgaagaaatacttgccaaactgaatgccggaaacggcaaactacaatctactgtagagcagataattacgctgccagatattatgctgcactcttttgctcaggtaaaagaactggcaggagacaagttaagttggggtgagaaaaacttcctttatcagcaggctcagaaacagctgaaagaaaataaaatggcggaatcccgcattctcagccgtgccaacccgcaactggcaaatgctgtccggatgggcatccgtcagtctgcgatgctgggtagctatgacgacctgttcccgcagcgcgccagccgctttgttaagccgggtgcggtggcctcaatgttttcaccggctggttatctgaccgagctgtatcgggaagcccgaggcctgcacgacgacacgtcagactatcatctggatacccgccgtccggacctggcatcaatggtgttgtctcagtcaaatatggacactgagttgtccaccctgtcgctctccaatgaactgttgctgaagttaattcagtcaaaggaaagcctgaattatgaccaggttattgaaaagctggcgacttacagactgaccggcaccacgccttacaatcaaccctatgaaaccatccgtcaggctattttgctgcaggacccggagtttaacgcattcagtaataatccggcagtggccgtaaaaatcaacaccagcgggctattaggtattacttccgatatcgccccggagctgcatgcgatactgactgaagagataacagaaaaaaaaacggaagcactgattaaaaagaacttcggcgatgccaatatcaaccagttccaaaatcttgcgtggctggcccactggtacggcttgtcctatgaggagcttaataacctggtaggcatgatttggtccagagatgatcttgaccccgctgttgagcactataaaaattccagcctggtcactttggtggctgaagacggtggatcgcttaacgcggtgttgattaagcgtactaaaggccatgattccgatgatatgcattatgcggaattaattcctgtgggaggagacaaatttcagtacaacttcagccttattgatgctgaaagcagtagtgtttattatcaattcggtacaaaaggaaagaactcccaagatttagttcctgtaatccatgagcctttgctgggtaatactccctatgctgttacattcacacttacacaagagcagctaagtaacccagttgaaatatccctgacgcatggtagtggcggtggtgatcgccttacctcaacaattttcactgttacgacttacccatttgataccttcctgctgaagctgaataaactcatacgcctctataaagccaccggtatctccccggccagcatcaggaccgtgattgaaagcgataacactgaccttatcatcacagaaagcgtattaaaccagctattctggactaattactatacacaaacgttcgaaatggaattttctgccgcactggtgctggcaggagcggacatcggtcagatagcacacagtgaaagccagccaagtgcgttcacccgcctgtttaacacaccgttgctggataaccagcagttttcggccagcgacgagtcactggatctggagccgggtaagggagccgatgctttccgtatcgctgtactcaagcgtgcattgcaggtgaatgacgccggactgtataccctttatggtctgagtttcaccgataaagataaaaacggtaagttgattccgttcaccaccaatattgagaacctttctgccctctatcgcacccgactgctggccgacatatttaatatttctgttactgagctgagcatgctgctgtcggtttcaccttatgccagtcagaaggtggacagccttaaaggtcaggcactatatcagtttgttgctaccctcagtgactatatgcaacggctgaaagcgatgaactggagcgtcagcgatctctacctgatgctgaccaacagctacagcacggtactgtcgccagaaattaaaaacctgatgactaccctgaaaaatggactcagcgagcaggattttaataacacggatgaaatcgctcagctgaatgcgacggcacctttaatcgccgcagcgatgcagcttgacttcacagaaaccgcagcagcactgctggaatggcttaatcaattgcaaccagcagggctgacagtggcaggtttcctgtctcttgtgaatcagacgacactcgaagataaggatgttgtaaaactggtctctttctgccaggttatggggcagcttgcactgatcgtgcgcaaggcggctctgggctccagcgaaatcacctttgcagttgcgcatccggctatttttaaaaaagatgcgaactcactggctcaggatattggcacgctctttgacctgacccagctgcatgcatttctgacagactgtggtacttatgcctctgaaattctcacctcactgaatgaagggaatctcgacgttagcacggtggcgacggcgctgacgctggacaaaacttcactggcgcaggcacttgctcaggtttcagaatctcaggccttttctaactggcacgaactgcgtgatgcacttcagtggacagatgccgccagcattttcaacatcacaccagtggctctgactgcgatggtgaacctgaaattcagcggtgacaactcttctccgtatcaggagtgggtaacggtcagcaaagctatgcaggtcgggctgaatcagacgcaaagcgctcagctgcaagcctcgctggatgaatccctcagcgcagcactcagcgcctacgtcattaagaacataacacccccatcagtaactgatcgcgacgaactttacggctggctgctgattgacaatcaggtctctgcacagattaaaactacccgcattgctgaagcgattgccagcgttcagctttacgtaaaccggtcactgacgggtcaggaagatggcgtggatagcaaggttaaatccggccagttctttacggcagactgggatacttataacaaacgctacagtacatgggccggtgtgtcggagctggtctattatccggaaaactatgttgatccgacgctgcgtatcgggcagaccgggatgatggatgaaatgttgcagacgctcagccagagccagattaatttagacaccgtcagtgatggtatggggcgttaccttactgattttgaagaaatagcaaatctaaaattcctcagtggttatcatgataatgtttctggccgtcaggggaaaacctggtttatcggtggcagtcagtctgaaccccaaaaattttactggcgatccctggattacagtaaaggcgatggggaggaattcgctgccaatgcatggtcagaatggaaccatatctcatgtgcaataacacccttacctggttttgttcgtgtggttttatttaactcccgactatatcttgcttgcgtggaaaaaaaagaaattcgggatagtgaaaacaaaaataaagcatcgtatcaattaaagatagctcacatcctttacaatggtgagtggagcgctcccttctcacacgatattactgatttatatgaggcaggctttgatccgagtacaacagtaatgcacttatctgtacatgatgagagtgatgcaatagtttgtatatttaataacagcgcgctagaaagtgacaaaaataaaggggtggcagtcaatgctgatatgtcatttaacaacattgacagcaaaagagtagatcagataattagtcttttagttcctgatcgttttatagatgaaggtaatgttatagataatttagtttctgagttaaagggatcggaagtcacggaaaataaaaaaacgctggagaatgattcgttcactatagatggatcaataaatttgaataagcattctatcgatatcacagggaaggccaatttagatattcaggcatcaattgctgtgcgtagtaaagcatctcctactagccatgagcgcgagctaataggctggttagatgaatctcaatttgattacattcgattattcaggggtggctataattttggccagaacgacggcattttggaatcatgcatgatttcggcagttaatagtgcctatacctgcttccttttacgagctgaccacttcagtggtttatttagttatggatatgacctttttgtattcaacggtgacgggtcaaaaacatatacacctcaagtgttgtttgaagatgatattcaagggactatggtgctcaagatcgtgctcctaaatgaggataaaaattcaaaactggaaaactttgaatccctggggcttatgaaaacatcagcaggcgatcatcagggagaaatagtttgcgaacttgctaaaagaaggacacctgagccttactgtgtagaattgagtcgctacttaccctcgaatgttactgttaccgttacatcaccatcggggaactttactgccaaagactatgtgttacctcttcccgcattcaataatggcgacgctgactataaattcgcaccattccccctctcgcttgaaagtatatggggagatggaaaaagtaccagtcgggacattaagtttacaataagcgtaaaagatacttgcggcaaggtggccacctcagagctaatctttacactttataaaaacacctcgcctgaattaattacactgaaaacgagtgacgcgggagcgcagtatatgcagcagggagtgtaccgcacaaggcttaataccctgtttgcacagaaattgatcaagcgtgttagcgccggaattgatgcagtgctgtcgtgggaaacccagcagttgcaggagcctaaactgggtactggcagttacatttcagtgcttatccccgcctatatcaaacttgagcacggagatagcagacaggctaacctgcagtttagtaatgtcgatcaaacaggaccggataatgggaattatatattatggtccggctcattaaatgacactccgcagcaggtcacgatttttgtgcccacgatgcaaactattggcgagctgcaattcccttatgaccggactagtggcctgaatctgagtttagcatgtgcagctggagtttatttgcaggggacattcaagaatatatctgcgtccgatttatctttaactgagtttgttgctgcaaagaacaatgactctaaacgggatgtcgaagtgacagtattaacttcaatcaatacggagccaatggacttcaagggtgccaacgccctctatttctgggagatgttctactacctccctatgatggtgtttaaacgccttctcagcgaaagtcggtttactgaagccactcagtggataaggtacgtctggaacccggacggctacctggtaaacgacacgcccgccacctaccagtggaacgtgcgcccgctggaggatgaaacctcctggcacgctaacccgctggactccgtggacccggatgccatagcccaggctgacccgctgcactacaaggtcgccacctttatggcgtaccttgacctgctgattgcccgcggcgacgcggcctaccgtcagcttgagcgcgatgcgctcagcgaagcaaaaatgtggtacgtgcaggcgctggacacccttggcgatgagccgtacctgagccagaacacaggctgggcgtccccatgcctgacggatgctgccgatgagaccacccataaaaacaggcagcaggcaatgctgaccgtgcgccagaaggttgcctccagcgaactgcgcaccgccaactccctaaccgccctgttcctgccacagcagaacgcgaagctggcaggctactggcagacgctgaaccagcgcctgtataacctgcgcaacaacctctccattgacggtaacccgctgtcgctgtccatttatgccaccccgactgacccggcggcgctgctcagctcggcggtgattagttctcaggggggcagtgacctgccagcggccgttatgccgctgtaccgcttcccggtgattctggaaagcgcacggagcatggtgaatcagctgacccagttcggcagcacgctgctcggcatcaccgagcgtcaggatgcagaggcgctgtctgatctgctgcagacacagggggctggactggcgctgcaaagcattgccctgcagaacagtaccatcagcgagattgatgcggatagggccgcgctcagggagagcctcagtggcgcacagtcgcgcctcaacagctataccaccctgtatgatgaaaatgttaatgctggtgaaacgcacgccatgaacctgtttctttcctccgccatcctggcagatggcgggcaggcctatcataccgccgcgggtgcgcttgacctggcgccgaatatctttggcctggccgacgggggttcccgctggggtgcggcatttaccgcaatggccggaatagctgatttggccgcctcggccacccatacggccgccgaccgcatcagccagtctgaggcataccgccgccgccgccaggagtgggaaatccagcgcaacgcggcgcagttcgaggtcagccaaatcaatgcccagctggacgcgctggcggtgcgtcgtgaaagcgccgtgctgcagaagacctatctggaaacacagcagggccagatgcaggcgcagatgaccttcctgcagaacaagttcaccagcaaagcactgtataactggttgcgcggtaaactggcggccatctactatcagttctatgacctgaccgtttcacgctgtctgatggcagaagctgcctacagctgggagatgaaaggctctcaggatacgggcacctttatccgtcccggcgcctggcagggaacctatgccggcctgatggcaggggaaacgctgatgctgaatctggcacagatggaaaacagctatctgacaaaagaggagcgccagaaagaggtcacgcgcacggtctgcctgtctgaagtttatgcagggctctcttcgggttcgttcgcgctggctgataccgtcaccacactggtgggtagcgggaaaggcaccgccggcacgaacgataacggagtgaagatcgatggcaagcagcttctggctaccctgaaactctccgatctgaacattaagacggattatccagagtcactggacaaagccaaacgcattaagcaaatcagcgtgacgctgccgatgctggtcgggccgtatcaggacgtccgggcggtactgagctatggcggcagcgtggttctgccacgcggctgcacggcggtcgccgtttcgcacggcatgaacgacagcggccagttccagctggacttcaatgacagccgctggctgccttttgaaggtatacctgttgatgattccggtacgctgacgctcagcttcccggacattaccgataagcaacaggaaaatctgctgctcagtctgagcgacatcatcctgcacatccgctataccatcgcaagctga

SEQ ID NO: 17 - DNA, 4266 bp, Pantoea agglomerans
  source 1..4266: mol_type = genomic DNA
atgcaaaatacagatcagatgagcctgacgcccccttccttaccctcaggtgggggtgccgtcaccggactgaaaggcgatatgtcaggagccggacccgatggcgccgccacgctgagccttcccctgccgatcagccccggacgtggctatgccccgtcgctgtcactgggttaccacagtcgtaacggcaacggtgtttttggcgcaggctggagctgcggtcagatggctattcgcctccaaacccgcaaaggcgtgccgttttatgacggcagcgacgtctttaccgctcctgatggtgaggttctggtgccggcgctggacgccagcggcaaggctgaggttcgcacgaccactacgctgctcggcgaaaacctcggcggcacctttaccgtacagacctaccgttcccgagtggaaaccgacttcagtcgcctggagcgctgggttccgcagaccgacgcagcggctgatttctggttgatttatagtccggacggccagatccacctgctgggtcgtaacccgcaggcgcgggtgaacaaccctgaggatacaacccagaccgccgcctggctgatagagtcgtcggtctccgccagcggcgagcagatttactggcaataccggcaggaagatgagctgggctgtacgcaggatgagaaaacggctcacgcacacgcgctcgcccagcgctatctggtggcggtatggtatggcaataaagcggccagccggacgctgccggggctgctgtctgttcctgcggctggcagctggctgtttacgctggcgctggactacggtgagcgggcgacagatcctgcaacaccaccggcctggctgtcaccgggcagtggcacatggctctgccggcaggatgtgttctccagctgggaatatggctttgagctgcgcacgcgtcgcctgtgccgacaggtactgatgtatcatgacgtcgcggcgctggcaggtcagtcaggttcagatgccgtgccacagctggtcaccagactgctgctggactataacacgtctccgtcgctgactaccctgaaaaccgcacagcaggccgcctgggaaccggatgggacgttgcgcagtctgccgccgctggcgttcagctggcagaccttcccgtcaacaccagagaaaagtgtcagctggcagcggcggaatgacatggggaaactcaacccacagcagccttatcagatggttgacctgcacggtgaaggactggcgggtatcctctatcaggacagtggtgcctggtggtatcgggagccggttcgtcagtcgggtgatgatgataatgccgtgacctgggcggctgcccgaccgctgccggcgttccctgctctgcgcaagggcggaatgctgctggatctggacggtgatggttacctggaatgggtggtcaccgcgccgggcgtcgcgggctgctatgcgcaagcgcctgaacaatactggcagcgcttcacgccgctgtctgcgctgccagtggaataccgccactcgcgaatggagatagccgacgtcaccggtgcgggtcttgcggatatgctgctgatcggcccgaaaagcgtacgcctgtacagcggcagcggcagaggctggaaaaaagcacgaacggtcatgcaggacagtggcatcaccctgccggttcccggtacaaatgcccgtgtcatggtggcattcagcgatatggccggcagcggtcagcaacacctgacggaaatcaaagccagcggcgtacgttactggcccagccttgggcatggtcgctttgcggctccggtgacactccccggcttcagtcagcccgctgaaaccttcaacccggcacagctctatctggccgacgttgacggttccggcaccaccgatctgatctatgctctgagcgatcatctgctggtatggctaaaccagagcggaaacagctttgacgcgcctttccgtatcagtcttccagaaggcgtgcgctatgacaatacctgcagtttgcaggtcgccgatattcaggggctgggcatctccagcctggtgctatcggtgccacatccgacgccgcgccattgggtatgtcacctgacgacggaaaagccctggctgctcgacggcatgaacaacaatatgggtgcccgccatactctctgttaccgtagttcggcgcagttctggctggatgaaaaggctgctgctaccgccgatcgacccgcgccggcgtgttatctgccgtttgcgctgcatacactgagccgtactgaagtcagtgatgaaatcaccggaaaccggctcaccaggacgatacgctaccggcacggggtctgggacaggcgcgagcgagagttccgcggctttggctttgttgaagtcagcgatgccgaagcgctggcaaaacaaactgaggggatgagcgcaccagcagttaaacgcagctggtatgctaccggactggcagccgtggatgcacagctcccggatgagttctggaaaggggatcatgcagcctttgccggttttacccctcgctttaccaccggcgatggcgaacaagaggcggcactggataccatcagcgacgatacccgtttctggctgacccgggcgattcgcggtacgctgctgcgtagcgaactgtatggcgcggatggcagcagccaggccgggatcccttacagcatcacggaatcgcggccacaagtgcggttgattactgaggcgggtaattcgccggtggtctggccctccgttatcgagaaccgcgccagtcattatgagcgcgtcagcagcgatccgcagtgcggccagcagatcctgttaaccagtaatgaatacggccagccgctccgtcagatcggcatcagttatccccggcgcaccaggcccgatgccagcccctacccggacgatctgccggacggactgtttgccgacagctttgatgagcaacagcaggcgctgcgcctgacgctgacacaaagcagctggcatacgctgaaagatatcagcagcggcatctggctgccggccgtggcggatgcaacccgaagcgatctgttcgttcaccaggcagcgcaggtgccgccagcgggtcttacgctggagaatttactcaccgatagcgcgctgctgaccagcccggtttttggcggacagtcgcaaatctggtatcaggacagggcgggtcaggcgagcatcacctcacccgattttcccccccgaccgtcctttagcgaaaccgcagcgctggacgaggcacaggtcagcacgctgtcagccgatattgatcaaacgaagctggagcaggcgggctatacccgctcagcgtatctgtttgcacgcagcggtgaggagagtaaaacgctgtgggcagtgcgccagggatatatcaccttcagcggcgcagaccatttctatctgccgattgccgcacagcagacgctgctggccggtaaaaccacagtcacctatgatccgtacgactgtgttgtcttacaggcaaaggacgccgcaggtgcggttacctccgcgacatacgactggcgttttctcgcgccgacgcagattactgatattaacgataatctgaaaagcgtcacgctggatgcgctgggtcgggtaacgtcgcagcgtttcagcggcactgaaaacggaaaaccggcgggctacagcgatcacgagtttccactgccggccagcgccgatgcagcgctggcgctcagtgccccgctaccggtggcacagtgcatcatctacgtaccggacagctggatgctgaccggggagcagcagcagccgccgcacgtggtaacgctgctcaccgaccgttacgacagcgacagtcagcagcagatccgtcagcaggttgttttcagcgatggttttggccgggtgctgcaggctgcctcaaggcaggtgaacggcgaagcgtggcagcgggcggcaaacggctcgttcgttgccggcacgaacgattcgcccgtgctgactgagacaacgttccgctgggccgttaccggacgcactgaatatgacaataagggacaggccatccgtgcttatcagccatattttctggacagctggaaatacgtgcgtgacgacagcgcgcgacaggatctgtacgccgacacccactattacgatccggtggggcgggagcggcaggtcattaccgcaaaaggctggctgcggcgcgtcactcacaccccctggttcgtagtcagcgaagacgaaaacgatacccaggcgtag

SEQ ID NO: 18 - DNA, 2856 bp, Pantoea agglomerans
  source 1..2856: mol_type = genomic DNA
atgtccgccgcgtatgtcttaagtaatttatcttataaactggagaatcctatgagcacctcgctttacagcaggaccccctcggtcacgatcctcgacaaccggggcctgtccgtacgcgatatcgcgtaccagcgccatccggataccccggcggtgaccagtgaacgcatcacccgccatcagtacgatgcccgcggctttctgatgcaaagcgccgacccgcgcctgcacgacgccgggctggcgaacgtcagctaccggaccaacctgaccggcagcgttctccgctcacagggcgtggataacggcatcaccgtgacgctgaacgatgccgccggacggccgtttctggcggtcagcaacatcagcactgccggtgatggcacggaggacagaagccaggcagtgacccgtacgtgtcagtacgaggacgccaccctgcccggacgtccgttaagtattacggagcaggtgaatggtggagccgcccgcatcacggagcgcttcgtctatgccggtaacgctgttgaggagaaagccctgaacctcgccgggcagcccgtcagccactatgataccgccggtctgacacagacagacagcatcgccctgaccggcgtgccgctctccgtcacccgccgcctgctgaaggacgcagacaatcctgacgccgtggctgactggcagggaacagacgcctccgtctggaacgacccgctcgacgtggaaacatacactaccctgtccacggcagacgccaccggcgcggtgctgaccaccaccgatgcgaagggaaacctgcagcggctggcctacgacgtggcgggcctgttgtcgggcagctggctgacgctgaaggatggcacggagcaggttatcgtgacgtccctgacctactccgccgccgggcagaagctgcgcgaggagcacggcaacggcgtggtgaccacctacacgtatgaagccgaaacgcagcgcctgaccggcattaaaacggcgcggccggccggacacgcctcaggtgcgaaggtgctgcaggacctgcgctacacctatgacccggtgggcaacgtcctgaaaatcagcaacgatgccgaagagacccgcttctggcgtaaccagaaagtggcgccggagagcgcgtacgtttatgacagcctgtaccagctggtcagcgccaccggacgcgagatggcgaacgccggtcagcagggcagcagctcatcgtcagccaccgtcccccttcccgccgacagttccgcgtttacaaactatacccgcaactatacttacgatgaggccggcaacctgacgcaggtccgtcataccccggctacgggcagcggctacaccacaaaaataaccgtctctgataaaagcaaccggggtgtgctgagcacgctgacggaaaatccctccgacgttgacgcgctgttcacggcgggcggccagcagaaacagctgcagccggggcagagtctcatctggacgccgcgtaacgagctgctgaaggtgacgccggtagcacgtgacggcggtgcggatgacagcgaaagctaccgctacgacgggggcagcctgcggctgctgaaggtcagcgtgcagaaaaccgggaacagcacgcagacgcagcgggcgctgtacctgccagggctggagctgcgcaacacaacatccggtgatacggaaacggagagcctgcaggtggttaccgtgggtgaagcggggcgcgcgcaggtgcgggtgctgcactgggagagcggaacgccggacagtgtcagcaacgacccggtgcgttacagctacgataacctgaccggcagcagcgggcttgagctggacagcagcggcaatattatcagcatggaggaatactatccgtacggcggcacggcggtctggacggcgcgcagcgcggtggaggcggagtacaaaaccgtgcgctactcgggcaaggagcgtgacgccacggggctgtactactacgggtaccggtactaccagccgtgggctggccgctggctgagcgcggacccggcgggcacggtggacgggctgaatttgttcaggatggtaaggaataacccggtaacattggttgatgataatggtttattcacgtcctcccctttattggggatttatgaaaaggagatgaaaacctttgatagtatcaaattgtcgattggttcttataaatacaaaccatctaaatttgatgaaaagaaaggtaagtatgttagctcagataaatacaaactgataatggcagatgataacgatcttaatgggtatttatttgacgagcgcgagatgacaagccatctaaaggactatgctgataagttcagtaaaataagcaggctaaatataggcgatgagcggatgaaaaccaatattaattttgggactagaatatcaagatatttgctatcttcagcacaagcatcatcacgcgaaaatcgtgaagtagatgttttgtcattcgaaagaaaattttttgctgtagtaaagaaaaaagataaaagtcattattttggacgaaaaatatatgccataggagaagctcatgtactaacagattttgaagagaaaaaaagaaccattgccattaagactctagttgcgcacccctatacgcaaattaatgaaagcattaaaaatagaattaatgattttgataaagaatataacgttaaagggattggaacttttgcaacgtttaaagctacgaacaagctcataggtggtattaagggagctttaaaatataagactaaagtgttgactcaagcggtaaatgtacgctcggcagctatagcaataaagtatggggcaaagcacgttccgtaa

SEQ ID NO: 19 - protein, 2522 aa, Pantoea agglomerans
MYLTEEILAKLNAGNGKLQSTVEQEITLPDIMVRSFAQVKELAGDRLSWGEKNFLYQQAQTQLKENKMAESRILSRANPQLANAVRLGIRQSSMLGSYDDLFPQRASRFVKPGAVASMFSPAGYLTELYREARGLHKAESQYNLDNRRPDLASLTLSQSNMDDELSTLSLSNELLLKLIQSKESLTYEQVMEKLATYRLTGTTPYNQPYEAIRQAILLQDPEFNAFSNNPAVAAKINTSGLLGITSDIAPELHAILTEEITEENAEALVKKNFGDVNIKQFQNLAWLANWYGLSYEKLNNLVGMIWSRDDLDPAIEHYKNSSLVTLVAEDGGSLNAVLIKRTKGHDSDDMHYAELIPVGGDKFQYNFSLIDAEASSSYYQFGTKGKYSQDLVPAIHKPLLGNTPYAVTFTLTQEQLSNPVEISLTHGSGGGDRLTSTIFTVTTSPFDIFLLKLNKLIRLYKATGISPASIRTVIESDNTDLIITESVLSQLFWTNYYTQTFEMEFSAALVLAGADIGQIAHSESQPSAFTRLFNTPPLDNQQFSASDESLDLEPGKGADAFRIAVLKRALQVNDAGLYTLYGLSFTDKDKNGELIPFTTKIENLSALYRTRLLADIFNISVTELSMLLSVSPYASQKVDSLKGQALHQFVTTLSDYMQRLKAMNWSVSDLYLMLTNSYSTVLSPEIKSLMTTLKNGLSEQDFNNTDEIAQLNATAPLIAAAMQLDSTETAAALLEWLNQLQPAGLTVAGFLSLVNQTTPEDKDVVKLVSFCQVMGQLALIVRKAALGSSEIIFAVAHPAIFNKDANSLAQDIGTLFDLTQLHAFLTECGTYASEILTSLNEGNLDVSTVATALTLDKTTLAQALAQVSESKAFSNWHELRDALQWTDAASIFNITPVALTAMVNLKFSGDNASPYQEWVTVSKAMQAGLNQTQSAQLQASLDESLGAAVSAYVIKNSSPSWVTDRDKLYSWLLIDNQVSAQVKTTRIAEAIASVQLYVNRALSGLENGQSITDAVDNAVKSGVFFTRDWDTYNKRYSTWAGVSELVYYPENYVDPTLRPGQTGMMDEMLQTLSQSQLTSDSVEDAFKTYMTRFEEIANLDIVSGYHDNLNDQKGVTYLIGRSAAGDYYWRSADISKLSDGKLPANAWAEWKKITTALTPVNNLVRPVIFQSRLYVTWVESREVGISADKEHNSETKILEYALKYAHILHDGTWSAPVSVKLENGTLPLDSVAIDVTGMYCAKDTQHDQLYILFYKKKETYNDVNDVLKGIILHDDGTTTITSGNSVSGLVVYKQLDTTKEVRLNTPYPGGKTYYSINNMRESSKWGDDNISMLSGCSVKDFVFTEGDGKLNVAFNATERIIYRGNPDSQGYVALVNMIKAIGNIGDTFKIPVLNSNGEGLDKPFTCIFRQPDEKTDAIAYFSDVQGLNIDHFAFNDESQKMLGRILRPEEKDFYKLECVNTNLHIYKDSSKTIKPDNFVYFGPGMDLIVVKGMIVETLFGLFGELKTGIKDKSVKLSVSAGVIDNSPAATKTKYTFDESLYVIEGQTVSIQLSEFKENNIDLEFTFFASGDSGNSLGKSVISATLTRTSENTIPVISLNKTSDNAQYLQYGIHRIRVNTLFAKQLVARANAGLDTVLSMATQQLTEPKMGKGAYIDLELNASSDGSSAVFEVLMCDVFTKGDRIALTSGTLSPTARTSCSFFIPRLGESTESPWNMYFCVKTQNDESKRVEVMGGEGKWSYQYVDESGTAIKPPYTDPYIASVYVRNDTTEPMDFNGANALYFWEMFYYVPMMVFKRLLSESKFAEATQWIKYIWNPDGYLVNNQPATYSWNVRPLEEDTSWHADPLDSVNPDAVAQADPLHYKVATFMAYLDLLIARGDAAYRQLERDTLNEAKMWYVQALNILGDEPYQSSSSGWSSPVLSSAAAQTTEKNVQQAMLAVRQQPDAGELRTANSLTDLFLPQQNAKLAGYWQTLAQRLYNLRHNLSIDGSPLSLAIYAAPADPAALLSAAVNSASGGSDLPAVVMPLYRFPVILESARGMAGQLIQFGSTLLSIAERQDAEALSELMQTQGSQLILQSIALQNSTISEIDADKTVLEASLSGARSRLDRYTTLYDEDVNTGEQQAMDLFYASSLQANGGQMFHTIAGALDLVPNIFGLADGGSRWGAVSTAMASIADLSAAACHTTAERLSQSEVYRRRRQEWEIQRNAAQSEIDQIDAQLASLTIRREGAVLQKTYLETQQGQMQAQMTFLQNKFTSKALYNWLRGKLAAIYYQFYDLTVSRCLMAEAAYSWDIKGNQETGTFIRPGAWQGTYAGLMAGETLMLNLAQMENSYLTKDERLKEVTRTVCLSEVYAGLSSDSFALADTVTTLVSNGKGNAGTDDNGVKIDDKQLLATLKLSDLSIDNDYPESLGKTRRIKQISVTLPTLVGPYQDVRAVLSYGGSVALPRGCTAVAVSHGMNDSGQFQLDFNDSRWLPFEGIPVGDSGTLTLSFPDITDKQQENLLLSLSDIILHIRYTIAS

SEQ ID NO: 20 - protein, 1421 aa, Pantoea agglomerans
MQNTDQMSLTPPSLPSGGGAVTGLKGDMSGAGPDGAATLNLPLPISPGRGYAPSLSLGYHSRNGNGVFGAGWSCGQMAIRLQTRKGVPFYDGSDVFTAPDGEVLVPALDASGKTEVRTTTTLLGENLGGTFTVQTYRSRVETDFSRLERWVSQADAAADFWLIYSPDGQIHLLGRNPQARVSNPEDTTQTAAWLIESSVSASGEQIYWQYRQEDELGCTQDEKTAHAHALAQRYLVAVWYGNKAASRTLPGLLSVPAAGSWLFTLVLDYGERTTDPATPPAWLSPGSGTWLCRQDVFSSWEYGFELRTRRLCRQVLMYHDVAALAGKPGSDAVPQLVTRLLLDYNLSPSLTTLKTAQQAAWEADGTLRSLPPLAFSWQTFPSTPEKSVSWQQRNDMGKLNPQQPYQMVDLHGEGLAGILYQDSGAWWYREPVRQLGDDDNAVTWAAARPLPAFPALRKGGMLLDLDGDGYLEWVVTAPGVAGCYAQTPEQCWQRFTPLSALPVEYRHSRMEITDVTGAGLADMLLIGPKSVRLYSGSGRGWKKARTVMQDSGITLPVPGTNARVMVAFSDMAGSGQQHLTEIKASGVRYWPSLGHGRFAAPVTLPGFSQPAETFNPAQLYLADVDGSGTTDLIYALSDHLLVWLNQSGNRFDEPFRIDLPEGVRYDNTCSLQVADIQGLGISSLVLSVPHPTPRHWVCHLTAEKPWLLDGMNNNMGARHTLCYRSSAQFWLDEKAAATADRPAPACYMPFALHTLSRTEVSDEITGNRLTRTIRYRHGVWDRREREFRGFGFVEVSDAEALAKQTEGMSAPAVKRSWYATGLTAVDAQLPDEFWKGDHAAFAGFTPRFTTGYGEQEAALDTISDDTRFWLTRAIRGTLLRSELYGADGSSQAGIPYSITESRPQVRLITEAGNSPVVWPSVIENRASHYERVSSDPQCGQQILLTSNEYGQPLRQIGVSYPRRTRPDASPYPDDLPDGLFADSFDEQQQALRLTLTQSSWHTLKDISSGIWLPAVADATRSDLFVHQAAQVPPAGLTLENLLTDSALLTSPVFGGQSQTWYQDSAGQASTTSPDFPLRPSFSETAALDEAQVSALSADIDQTKLEQAGYTRSAYLFARSGEEGKTLWTVRQGYITFSSADHFYLPIAAQQTLLTGKTTVTYDPYDCVVLQAKDAAGAVTSATYDWRFLAPTQITDINDNLKSVTLDALGRVTSQRFSGSENGKPAGYSDDAFPLPASADAALALSAPLPVAQCIIYVPDSWMLTGEQQQPPHVVTLLTDRYDSDSQQQIRQQVVFSDGFGRVLQAASRQVNGEAWQRAANGSFVADPNGSPVLTETTFRWAVTGRTEYDNKGQAIRTYQPYFLDSWKYVRDDSARHDLYADTHYYDPVGRERQVITAKGLLRRVTYTPWFVVSEDENDTQA

SEQ ID NO: 21 - protein, 1004 aa, Pantoea agglomerans
MSAAYVLSNLSYQRENTMSTSLYSRTPSVTVLDNRGLTVRDIAYHRHPDTPAVTSERITRHQYDARGFLTQSADPRLHDAGLANFSYRTDLTGSVLRLQGVDNGITVALNDAAGRPFLAVSNIRTAGDGSEDRSQALTRTCQYEDATLPGRPLSITEQVKGGAARITERFIYAGNAVEEKALNLAGQPVSHYDTAGLTQTDSIALTGVPLSVTRRLLKDADNPDAVADWQGTDASVWNDPLDVETYTTLSTADATGAVLTTTDAKGNLQRLAYDVAGLLSGSWLTLKDGTEQVIVTSLTYSAAGQKLREEHGNGVVTTYTYEAETQRLTGIKTARPAGHTSGAKVLQDLRYTYDPVGNVLKISNDAEETRFWRNQKVVPESAYVYDSLYQLVSATGREMANAGQQGSSSSSATVPLPADSSAFTNYTRNYTYDEAGNLTQVRHTPATGSGYTTKITVSDKSNRGVLSTLTENPSDVDALFTAGGQQKQLQPGQSLIWTPRNELLKVTPVARDGGADDSESYRYDGGSLRLLKVSVQKTGNSTQTQRALYLPGLELRNTTSGDTETESLQVVTVGEAGRAQVRVLHWESGTPDSVSNDQLRYSYDNLTGSSGLELDSSGNIISMEEYYPYGGTAVWTARSAVEAKYKTVRYSAKERDATGLYYYGYRYYQPWAGRWLSADPAGTADGLNLFRMVRNNPVTLKDTNGLISTGQDARKLVAEAFVHPLHMTVFERISSEENLAMSVREAGIYTISALGEGAAAKGHNILEKTIKPGSLKAVYGDNAESILAQAKRSGFVGRVGQWDASGVRGIYAHNTPGGEDLAYPVNLKNSSANELVNAWIKFKIITPYTGDYDMHDIIKISDGKGHVPLAESNEEKGVKDMINEGVAQVDPARPFTSTAMNVVRHGPQVNFVPYMWEHEHENVVRDNGYLGVVARPGPFPVAMVHKGEWTVFDNKNELFEFYKSTNTPLPEHWSQDFVERGKGNVATPRHAEILDRNSSRLRAA

SEQ ID NO: 22 - protein, 971 aa, Pantoea agglomerans
MCSVADFDRLHNIKQENIMSTSLYSRTPSVTVLDNRGLTVRDIAYHRHPDTPAVTSERITRHQYDARGFLTQSADPRLHDAGLANFSYRTDLTGSVLRLQGVDNGITVALNDAAGRPFLAVSNIRTAGDGSEDRSQALTRTCQYEDATLPGRPLSITEQVKGGAARITERFIYAGNAVEEKALNLAGQPVSHYDTAGLTQTDSIALTGVPLSVTRRLLKDADNPDAVADWQGTDASVWNDLLGAETYTTLSTADVTGAVLTTTDAKGNLQRLAYDVAGLLSGSWLTLKDGTEQVIVTSLTYSAAGQKLREEHGNGVVTTYTYEAETQRLTGIKTERPAGHASGAKVLQDLRYTYDPVGNVLKISNDAEETRFWRNQKVVPENAYVYDSLYQLVSATGREMANAGQQGSSSSSATVPLPADSSAFTNYTRNYTYDEAGNLTQVRHTPATGSGYTTKITVSDKSNRGVLSTLTENPSDVDALFTAGGQQKQLQPGQSLIWTPRNELLKVMPIMRDGGTDDSESYRYDGGSQRLLKVSVQKTGNSTQTQRALYLPGLELRTTKSGDTLTESLQVITAGEAGRAQVRVLHWESGTPDSVSNDQLRYSYDNLTGSSGLELDSSGNIISMEEYYPYGGTAVWTARSAVEAKYKTVRYSGKERDATGLYYYGYRYYQPWAGRWLSADPAGTVDGLNLFRMVRNNPLTLKDNDGLKPINENFRENKGDLVYGLAAPRGAYISTAIGRKFAPEEKDAPASIIDLYNNTVSGQALLSVDFKILQDFMKSPKKNEKKLAPPSNIKELVKKSRDYPLWEDYFLAGENNPKFNIASIYKEVRKDAGKTQYHEWHIAGGQSAPKLLWKRGSKLGIEMAASGAGNKIHFVLDELDISNVVNKEGPGGKSITASELRYAYRNRERLTGNIHFYKNNAETGAPWDTNAELWASYHPKPKHKGNESTHIMSQRRNGSLFKSMRKVFSRN

SEQ ID NO: 23 - protein, 2523 aa, Pantoea agglomerans
MYLTEEILAKLNAGNGKLQSTVEQIITLPDIMVRSFSQVKELAGDKLSWGEKNFLYQQAQTQLKENKMAESRILSRANPQLANAVRLGIRQSSMLGSYDDLFPQRASRFVKPGAVASMFSPAGYLTELYREARGLHKAESQYNLDKRRPDLASLALSQSNMDDELSTLSLSNELLLNNIQQHDGLSYDDALKKLAGYRQTGTTPYSQPYETIREAILLQDPAFNSIRNNPAVATKMNTSGLLGLTANLPPELHAILTETITEENAEQLIKDNFGDVNVSRFQDVSYLARWYGMTPYELNSVLGLMEVGSNPVDGVTYYQDDQLISLVDNGGNLDAVLMQRAGGDNYSQFGYIELLPVSGDTYQLRFTVQSGYVGQDSEVRIGTSENAGSKDILSDGRIAGLNIPMVLNVKLDSTKLAQGITIGVTRYDPSGSYINFASVRFQRYDFSYNVFLLKLNKIIRLYKATGISPSDIQTLIESANHDLAITEDVLSQLFWTNYYTQRYGIDFSAALVLAGANISQIAHSNKQSAFTRLFNTPPLNNQFFYADGKKLNLEPGKSDDSHGLGVLKRALQVNDSALYTLFNLTFADKDAQGNAVVFTKTPENLSALYRTRLLATVNNLTVNELSLLLSVSPYVKVKLATLKDEALSQLSTTLERYTQWLDKMNWTIGDLYLMLTPVYSTVLSPDIENLVTTLKNGLAGQDLTSDEKRIAALAPFVAAATQLDSAETARALLRWLNDLKPGTLSLADFIAQVNNTTQTENLVTFSQVMAQLALITRNASLSANELSWAVAHPEIFQEKATVLKNDIATLNDLTQLHDLLARCGSHASEILTSLSGNASKAENNLAVSTLATALNLDERALTQALAKVSTYEYFYNWAHLNEALQWLDVATTFGITPDNLAALIGLKFDNQDDASFASWLTASRFMQAGLNTQQTAQLSATLDESLSAAVSAYAIKNIFSGAVSNREQLYSWLLIDNQVSAQVKTTRIAEAIASVQLYVNRALSGLENGQSATDAVDNAVKSGVFFTRDWDTYNKRYSTWAGVSELVYYPENYVDPTLRLGQTGMMDEMLQTLSQSQLTSDTVEDAFKTYMTRFEEIANLDIVSGYHDNLSDQKGVTYLIGRSAAGDYYWRSADISKLSDGKLPANAWAEWKKITTALTPVNNLVRPVIFQSRLYVTWVESREVGISAVKKQNSETKPLEYALKYAHILHDGTWSAPVSVKLENGTLPLDSVAIDVTGMYCAKDTQHDQLYILFYKKKETYNDVNDVLKGIILHDDGTTTITSGNSVSGLVVYKQLDTTKEVRLNTPYPGGKTYYSINNMRESSKWGDDNISMLSGCSVKDFVFTEGDGKLNVAFNATERIIYRGNPDSQGYVALVNMIKAIGNIGDTFKIPVLNSNGEGLDRPFTCIFRQPDEKTDAIAYFSDVQGLNIDHFAFNDESQKMLGRILRPEEKDFYKLECVNTNLHIYKDSSKTIKPDNFVYFGPGMDLIVVKGMIVETLFGLFGELKTGIKDKSVKLSVSAGVIDNSPAATKTKYTFDESLYVIEGQTVSIQLSEFKENNIDLEFTFLASGDSGNSLGQSVISATLTRTSENTIPVISLNKTSDNAQYLQYGIHRIRVNTLFAKQLVARANAGLDTVLSMATQQLTEPKMGKGAYIDLELNASSDGSSAVFEVLMCDVFTNGDRIALTSGTLSPTARTSCSFFVPRLDESTASAYNMYFCVKTQNTESKRVEVTGGEGKWDYQYVDESGAAIKPPYTDPYIASIYVRNDTTEPMDFNGANALYFWEMFYYVPMMVFKRLLSESKFAEATQWIKYIWNPDGYLVNNQPATYTWNVRPLEEDTSWHADPLDSVNPDAVAQADPLHYKVATFMAYLDLLIARGDAAYRQLQRDTLNEAKMWYVQALNILGDEPYQSSSSDWSSPVLSSAADQTTEKNVQQAMLAVRQQPDAGELRTANSLTSLFLPQQNEKLAGYWQTLAQRLYNLRHNLSIDGSPLSLAIYAAPADPAALLSAAVNSASGGSELPAAVMPLYRFPIILESARGMAGQLTQFGSTLLSIAERQDAEALSELMQTQGSQLILQSIALQNSTISEIDADKTVLEASLSGARSRLDRYTTLYDEDVNTGEQQAMDLFYASSIQANGGQAFHTVAGGLDLAPNIFGLADGGSRWGAAFTALASIADLSAAASHTAAERLSQSEVYRRRRQEWEIQRNAAQSEIDQIDAQLASLTIRRKGAVLQKTYLETQQGQMQAQMTFLQNKFTSKALYNWLRGKLAAIYYQFYDLTVSRCLMAEAAYSWHIKGNQETGTFIRPGAWQGIYAGLMAGEALMLNLAQMENSYLTKDERLQEVTRTVCLSEFYSGLSSNKFALADTVTTLVNSGKGNAGTTDNGVKIDGKQLLATLKLSDLNIKTDYPESLDKAKRIKQISVTLPMLVGPYQDVRAVLSYGGSVVLPRGCTAVAVSHGMNDSGQFQLDFNDSRWLPFEGIPVDDSGTLTLSFPDITDKQQENLLLSLSDIILHIRYTIAS

SEQ ID NO: 24 - protein, 1421 aa, Pantoea agglomerans
MQNTDQMSLTPPSLPSGGGAVTGLKGDMSGAGPDGAATLSLPLPISPGRGYAPSLSLGYHSRNGNGVFGAGWSCGQMAIRLQTRKGVPFYDGSDVFTAPDGEVLVPALDASGKAEVRTTTTLLGENLGGTFTVQTYRSRVETDFSRLERWVPQTDAAADFWLIYSPDGQIHLLGRNPQARVNNPEDTTQTAAWLIESSVSASGEQIYWQYRQEDELGCTQDEKTAHAHALAQRYLVAVWYGNKAASRTLPGLLSVPAAGSWLFTLVLDYGERATDPATPPAWLSPGSGTWLCRQDVFSSWEYGFELRTRRLCRQVLMYHDVAALAGQSGSDAVPQLVTRLLLDYNTSPSLTTLKTAQQAAWEPDGTLRSLPPLAFSWQTFPSTPEKSVSWQRRNDMGKLNPQQPYQMVDLHGEGLAGILYQDSGAWWYREPVRQSGDDDNAVTWAAARPLPAFPALRKGGMLLDLDGDGYLEWVVTAPGVAGCYAQAPEQCWQRFTPLSALPVEYRHSRMEITDVTGAGLADMLLIGPKSVRLYSGSGRGWKKARTVMQDSGITLPVPGTNARVMVAFSDMAGSGQQHLTEIKASGVRYWPSLGHGRFAAPVTLPGFSQPAETFNPAQLYLADVDGSGTTDLIYALSDHLLVWLNQSGNSFDAPFRISLPEGVRYDNTCSLQVADIQGLGISSLVLSVPHPTPRHWVCHLTTEKPWLLDGMNNNMGARHTLCYRSSAQFWLDEKAAATADRPAPACYLPFALHTLSRTEVSDEITGNRLTRTIRYRHGVWDRREREFRGFGFVEVSDAEALAKQTEGMSAPAVKRSWYATGLAAVDAQLPDEFWKGDHAAFAGFTPRFTTGDGEQEAALDTISDDTRFWLTRAIRGTLLRSELYGADGSSQAGIPYSITESRPQVRLITEAGNSPVVWPSVIENRASHYERVSSDPQCGQQILLTSNEYGQPLRQIGISYPRRTRPDTSPYPDDLPDGLFADSFDEQQQALRLTLTQSSWHTLKDISSGIWLPAVADATRSDLFVHQAAQVPPAGLTLENLLTDSALLTSPVFGGQSQIWYQDRAGQASITSPDFPPRPSFSETAALDEAQVSALSADIDQTKLEQAGYTRSAYLFARSGEESKTLWAVRQGYITFSGADHFYLPIAAQQTLLAGKTTVTYDPYDCVVLQAKDAAGAVTSATYDWRFLAPTQITDINDNLKSVTLDALGRVTSQRFSGTENGKPAGYSDDEFPLPASADAALALSAPLPVAQCIIYVPDSWMLTGEQQQPPHVITLLTDRYDSDSQQQIRQQVVFSDGFGRVLQAASRQVNGEAWQRAANGSFVAGTNDSPVLTETTFRWAVTGRTEYDNKGQAIRAYQPYFLDSWKYVRDDSARQDLYADTHYYDPVGRERQVITAKGWLRRVTHTPWFVVSEDENDTQA

SEQ ID NO: 25 - protein, 1004 aa, Pantoea agglomerans
MSAAYVLSNLSYKLENPMSTSLYSRTPSVTILDNRGLTVRGIAYQRHPDTPAVTSERITRHQYDARGFLMQSADPRLHDAGLANVSYRTNLTGSVLRSQGVDNGITVTLNDAAGRPFLAVSNISTAGDGTEDRSQAVTRTCQYEDATLPGRPLSITEQVNGGAARITERFVYAGNAVEEKALNLAGQPVSHYDTAGLTQTDSIALTGVPLSVTRRLLKDADNPDAVADWQGTDASVWNDPLDVETYTTLSTADATGAVLTTTDAKGNLQRLAYDVAGLLSGSWLTLKDGTEQVIVTSLTYSAAGQKLREEHGNGVVTTYTYEAETQRLTGIKTARPAGHTSGAKVLQDLRYTYDPVGNVLKISNDAEETRFWRNQKVAPESAYVYDSLYQLVSATGREMANAGQQGSSSSSATVPLPADSSAFTNYTRNYTYDEAGNLTQVRHTPATGSGYTTKITVSDKSNRGVLSTLTENPSDVDALFTAGGQQKQLQPGQSLIWTPRNELLKVTPVARDGGADDSESYRYDGGSLRLLKVSVQKTGNSTQTQRALYLPGLELRNTTSGDTETESLQVVTVGEAGRAQVRVLHWESGTPDSVSNDPVRYSYDNLTGSSGLELDSSGNIISMEEYYPYGGTAVWTARSAVEAKYKTVRYSAKERDATGLYYYGYRYYQPWAGRWLSADPAGTADGLNLFRMVRNNPVTLKDTNGLISTGQDARKLVAEAFVHPLHMTVFERISSEENLAMSVREAGIYTISALGEGAAAKGHNILEKTIKPGSLKAVYGDNAESILAQAKRSGFVGRVGQWDASGVRGIYAHNTPGGEDLAYPVNLKNSSANELVNAWIKFKIITPYTGDYDMHDIIKISDGKGHVPMAESNEEKGVKDMINEGVAQVDPARPFTSTAMNVVRHGPQVNFVPYMWEHEHENVVRDNGYLGVVARPGPFPVAMVHKGEWTVFDNKNELFEFYKSTNTPLPEHWSQDFVERGKGNVATPRHAEILDRNSSRLRAA

SEQ ID NO: 26 - protein, 971 aa, Pantoea agglomerans
MCSVADFDRLHNIKQENIMGTSLYSKTPSVTILDNRGLSVRDIAYQRHPDTPAVTSECITRHQYDARGFLMQSADPRLHDAGLANFSYRTDLTGSVLRSQGVDNGITVTLNDAAGRPFLAVSNISTAGDGTEDRSQAVTRTCQYEDATLPGRPLSITEQVNGGAARITERFIYAGNAVEEKALNLAGQPVSHYDTAGLTQTDSIALTGAPLSVTRRLLKDADNPDAVADWQGTDASVWNDPLDVETYTTLSTADATGAVLTTTDAKGNLQRLAYDVAGLLSGSWLTLKDGTEQVIVTSLTYSAAGQKLREEHGNGVVTTYTYEAETQRLTGIKTARPAGHTSGAKVLQDLRYTYDPVGNVLKISNDAEETRFWRNQKVVPESAYVYDSLYQLVSATGREMANAGQQGSSSSSATVPLPADSSAFTNYTRNYTYDEAGNLTQVRHTPATGSGYTTKITVSDKSNRGVLSTLTENPSDVDALFTAGGQQKQLQPGQSLIWTPRNELLKVMPIMRDGGTDDSESYRYDGGSQRLLKVSVQKTGNSTQTQRALYLPGLELRNTTSGDTETESLQVVTAGEAGRAQVRVLHWESGTPDSVSNDPVRYSYDNLTGSSGLELDSSGNIISMEEYYPYGGTAVWTARSAVEAKYKTVRYSAKERDATGLYYYGYRYYQPWAGRWLSADPAGTVDGLNLFRMVRNNPLTLKDNDGLKPINENFRENKGDLVYGLAAPRGAYISTAIGREFAPEEKDAPASIIDLYNNTVSGQALLSVDFKILQDFMKSPKKHEKKLAPPSNIKELVKKSRVYPLWEDYFLAGENNPKFNIASIYKEVRKDAGKTQYHEWHIAGGQSAPKLLWKRGSKLGIEMAASGAGNKIHFVLDELDISNVVNKEGPGGKSITASELRYAYRNRERLTGNIHFYKNNAETGAPWDTNAELWASYHPKPKHKGNESTHMMSQRRNGSLFKSMRKVFSRN

SEQ ID NO: 27 - protein, 2518 aa, Pantoea agglomerans
MYLTEEILAKLNAGNGKLQSTVEQIITLPDIMLHSFAQVKELAGDKLSWGEKNFLYQQAQKQLKENKMAESRILSRANPQLANAVRMGIRQSAMLGSYDDLFPQRASRFVKPGAVASMFSPAGYLTELYREARGLHDDTSDYHLDTRRPDLASMVLSQSNMDTELSTLSLSNELLLKLIQSKESLNYDQVIEKLATYRLTGTTPYNQPYETIRQAILLQDPEFNAFSNNPAVAVKINTSGLLGITSDIAPELHAILTEEITEKKTEALIKKNFGDANINQFQNLAWLAHWYGLSYEELNNLVGMIWSRDDLDPAVEHYKNSSLVTLVAEDGGSLNAVLIKRTKGHDSDDMHYAELIPVGGDKFQYNFSLIDAESSSVYYQFGTKGKNSQDLVPVIHEPLLGNTPYAVTFTLTQEQLSNPVEISLTHGSGGGDRLTSTIFTVTTYPFDTFLLKLNKLIRLYKATGISPASIRTVIESDNTDLIITESVLNQLFWTNYYTQTFEMEFSAALVLAGADIGQIAHSESQPSAFTRLFNTPLLDNQQFSASDESLDLEPGKGADAFRIAVLKRALQVNDAGLYTLYGLSFTDKDKNGKLIPFTTNIENLSALYRTRLLADIFNISVTELSMLLSVSPYASQKVDSLKGQALYQFVATLSDYMQRLKAMNWSVSDLYLMLTNSYSTVLSPEIKNLMTTLKNGLSEQDFNNTDEIAQLNATAPLIAAAMQLDFTETAAALLEWLNQLQPAGLTVAGFLSLVNQTTLEDKDVVKLVSFCQVMGQLALIVRKAALGSSEITFAVAHPAIFKKDANSLAQDIGTLFDLTQLHAFLTDCGTYASEILTSLNEGNLDVSTVATALTLDKTSLAQALAQVSESQAFSNWHELRDALQWTDAASIFNITPVALTAMVNLKFSGDNSSPYQEWVTVSKAMQVGLNQTQSAQLQASLDESLSAALSAYVIKNITPPSVTDRDELYGWLLIDNQVSAQIKTTRIAEAIASVQLYVNRSLTGQEDGVDSKVKSGQFFTADWDTYNKRYSTWAGVSELVYYPENYVDPTLRIGQTGMMDEMLQTLSQSQINLDTVSDGMGRYLTDFEEIANLKFLSGYHDNVSGRQGKTWFIGGSQSEPQKFYWRSLDYSKGDGEEFAANAWSEWNHISCAITPLPGFVRVVLFNSRLYLACVEKKEIRDSENKNKASYQLKIAHILYNGEWSAPFSHDITDLYEAGFDPSTTVMHLSVHDESDAIVCIFNNSALESDKNKGVAVNADMSFNNIDSKRVDQIISLLVPDRFIDEGNVIDNLVSELKGSEVTENKKTLENDSFTIDGSINLNKHSIDITGKANLDIQASIAVRSKASPTSHERELIGWLDESQFDYIRLFRGGYNFGQNDGILESCMISAVNSAYTCFLLRADHFSGLFSYGYDLFVFNGDGSKTYTPQVLFEDDIQGTMVLKIVLLNEDKNSKLENFESLGLMKTSAGDHQGEIVCELAKRRTPEPYCVELSRYLPSNVTVTVTSPSGNFTAKDYVLPLPAFNNGDADYKFAPFPLSLESIWGDGKSTSRDIKFTISVKDTCGKVATSELIFTLYKNTSPELITLKTSDAGAQYMQQGVYRTRLNTLFAQKLIKRVSAGIDAVLSWETQQLQEPKLGTGSYISVLIPAYIKLEHGDSRQANLQFSNVDQTGPDNGNYILWSGSLNDTPQQVTIFVPTMQTIGELQFPYDRTSGLNLSLACAAGVYLQGTFKNISASDLSLTEFVAAKNNDSKRDVEVTVLTSINTEPMDFKGANALYFWEMFYYLPMMVFKRLLSESRFTEATQWIRYVWNPDGYLVNDTPATYQWNVRPLEDETSWHANPLDSVDPDAIAQADPLHYKVATFMAYLDLLIARGDAAYRQLERDALSEAKMWYVQALDTLGDEPYLSQNTGWASPCLTDAADETTHKNRQQAMLTVRQKVASSELRTANSLTALFLPQQNAKLAGYWQTLNQRLYNLRNNLSIDGNPLSLSIYATPTDPAALLSSAVISSQGGSDLPAAVMPLYRFPVILESARSMVNQLTQFGSTLLGITERQDAEALSDLLQTQGAGLALQSIALQNSTISEIDADRAALRESLSGAQSRLNSYTTLYDENVNAGETHAMNLFLSSAILADGGQAYHTAAGALDLAPNIFGLADGGSRWGAAFTAMAGIADLAASATHTAADRISQSEAYRRRRQEWEIQRNAAQFEVSQINAQLDALAVRRESAVLQKTYLETQQGQMQAQMTFLQNKFTSKALYNWLRGKLAAIYYQFYDLTVSRCLMAEAAYSWEMKGSQDTGTFIRPGAWQGTYAGLMAGETLMLNLAQMENSYLTKEERQKEVTRTVCLSEVYAGLSSGSFALADTVTTLVGSGKGTAGTNDNGVKIDGKQLLATLKLSDLNIKTDYPESLDKAKRIKQISVTLPTLVGPYQDVRAVLSYGGSVVLPRGCTAVAVSHGMNDSGQFQLDFNDSRWLPFEGIPVDDSGTLTLSFPDITDKQQENLLLSLSDIILHIRYTIAS

SEQ ID NO: 28 - protein, 1421 aa, Pantoea agglomerans
MQNTDQMSLTPPSLPSGGGAVTGLKGDMSGAGPDGAATLSLPLPISPGRGYAPSLSLGYHSRNGNGVFGAGWSCGQMAIRLQTRKGVPFYDGSDVFTAPDGEVLVPALDASGKAEVRTTTTLLGENLGGTFTVQTYRSRVETDFSRLERWVPQTDAAADFWLIYSPDGQIHLLGRNPQARVNNPEDTTQTAAWLIESSVSASGEQIYWQYRQEDELGCTQDEKTAHAHALAQRYLVAVWYGNKAASRTLPGLLSVPAAGSWLFTLALDYGERATDPATPPAWLSPGSGTWLCRQDVFSSWEYGFELRTRRLCRQVLMYHDVAALAGQSGSDAVPQLVTRLLLDYNTSPSLTTLKTAQQAAWEPDGTLRSLPPLAFSWQTFPSTPEKSVSWQRRNDMGKLNPQQPYQMVDLHGEGLAGILYQDSGAWWYREPVRQSGDDDNAVTWAAARPLPAFPALRKGGMLLDLDGDGYLEWVVTAPGVAGCYAQAPEQYWQRFTPLSALPVEYRHSRMEIADVTGAGLADMLLIGPKSVRLYSGSGRGWKKARTVMQDSGITLPVPGTNARVMVAFSDMAGSGQQHLTEIKASGVRYWPSLGHGRFAAPVTLPGFSQPAETFNPAQLYLADVDGSGTTDLIYALSDHLLVWLNQSGNSFDAPFRISLPEGVRYDNTCSLQVADIQGLGISSLVLSVPHPTPRHWVCHLTTEKPWLLDGMNNNMGARHTLCYRSSAQFWLDEKAAATADRPAPACYLPFALHTLSRTEVSDEITGNRLTRTIRYRHGVWDRREREFRGFGFVEVSDAEALAKQTEGMSAPAVKRSWYATGLAAVDAQLPDEFWKGDHAAFAGFTPRFTTGDGEQEAALDTISDDTRFWLTRAIRGTLLRSELYGADGSSQAGIPYSITESRPQVRLITEAGNSPVVWPSVIENRASHYERVSSDPQCGQQILLTSNEYGQPLRQIGISYPRRTRPDASPYPDDLPDGLFADSFDEQQQALRLTLTQSSWHTLKDISSGIWLPAVADATRSDLFVHQAAQVPPAGLTLENLLTDSALLTSPVFGGQSQIWYQDRAGQASITSPDFPPRPSFSETAALDEAQVSTLSADIDQTKLEQAGYTRSAYLFARSGEESKTLWAVRQGYITFSGADHFYLPIAAQQTLLAGKTTVTYDPYDCVVLQAKDAAGAVTSATYDWRFLAPTQITDINDNLKSVTLDALGRVTSQRFSGTENGKPAGYSDHEFPLPASADAALALSAPLPVAQCIIYVPDSWMLTGEQQQPPHVVTLLTDRYDSDSQQQIRQQVVFSDGFGRVLQAASRQVNGEAWQRAANGSFVAGTNDSPVLTETTFRWAVTGRTEYDNKGQAIRAYQPYFLDSWKYVRDDSARQDLYADTHYYDPVGRERQVITAKGWLRRVTHTPWFVVSEDENDTQA

SEQ ID NO: 29 - protein, 951 aa, Pantoea agglomerans
MSAAYVLSNLSYQRENTMSTSLYSRTPSVTVLDNRGLTVRDIAYHRHPDTPAVTSERITRHQYDARGFLTQSADPRLHDAGLANFSYRTDLTGSVLRLQGVDNGITVALNDAAGRPFLAVSNIRTAGDGSEDRSQAVTRTCQYEDATLPGRPLSITEQVKGGAARITERFIYAGNAVEEKALNLAGQPVSHYDTAGLTQTDSIALTGVPLSVTRRLLKDADNPDAVADWQGTDASVWNDPLDVETYTTLSTADATGAVLTTTDAKGNLQRLAYDVAGLLSGSWLTLKDGTEQVIVTSLTYSAAGQKLREEHGNGVVTTYTYEAETQRLTGIKTARPAGHTSGAKVLQDLRYTYDPVGNVLKISNDAEETRFWRNQKVAPESAYVYDSLYQLVSATGREMANAGQQGSSSSSATVPLPADSSAFTNYTRNYTYDEAGNLTQVRHTPATGSGYTTKITVSDKSNRGVLSTLTENPSDVDALFTAGGQQKQLQPGQSLIWTPRNELLKVTPVARDGGADDSESYRYDGGSLRLLKVSVQKTGNSTQTQRALYLPGLELRNTTSGDTETESLQVVTVGEAGRAQVRVLHWESGTPDSVSNDPVRYSYDNLTGSSGLELDSSGNIISMEEYYPYGGTAVWTARSAVEAEYKTVRYSGKERDATGLYYYGYRYYQPWAGRWLSADPAGTVDGLNLFRMVRNNPVTLVDDNGLFTSSPLLGIYEKEMKTFDSIKLSIGSYKYKPSKFDEKKGKYVSSDKYKLIMADDNDLNGYLFDEREMTSHLKDYADKFSKISRLNIGDERMKTNINFGTRISRYLLSSAQASSRENREVDVLSFERKFFAVVKKKDKSHYFGRKIYAIGEAHVLTDFEEKKRTIAIKTLVAHPYTQINESIKNRINDFDKEYNVKGIGTFATFKATNKLIGGIKGALKYKTKVLTQAVNVRSAAIAIKYGAKHVP

SEQ ID NO: 30 - protein, 2523 aa, Pantoea agglomerans; identical to SEQ ID NO: 23

SEQ ID NO: 31 - protein, 1421 aa, Pantoea agglomerans
MQNTDQMSLTPPSLPSGGGAVTGLKGDMSGAGPDGAATLSLPLPISPGRGYAPSLSLGYHSRNGNGVFGAGWSCGQMAIRLQTRKGVPFYDGSDVFTAPDGEVLVPALDASGKAEVRTTTTLLGENLGGTFTVQTYRSRVETDFSRLERWVPQTDAAADFWLIYSPDGQIHLLGRNPQARVNNPEDTTQTAAWLIESSVSASGEQIYWQYRQEDELGCTQDEKTAHAHALAQRYLVAVWYGNKAASRTLPGLLSVPAAGSWLFSLVLDYGERTTDPATLPAWLSPGSGTWLCRQDVFSSWEYGFELRTRRLCRQVLMYHDVAALAGQSGSDAVPQLVTRLLLDYNLSPSLTTLKTAQQAAWEPDGTLRSLPPLAFSWQTFPSTPEKSVSWQRRNDMGKLNPQQPYQMVDLHGEGLAGILYQDSGAWWYREPVRQSGDDDNAVTWAAARPLPAFPALRKGGMLLDLDGDGYLEWVVTAPGVAGCYAQAPEQCWQRFTPLSALPVEYRHSRMEIADVTGAGLADMLLIGPKSVRLYSGSGRGWKKARTVMQDSGITLPVPGTNARVMVAFSDMAGSGQQHLTEIKASGVRYWPSLGHGRFAAPVTLPGFSQPAETFNPAQLYLADVDGSGTTDLIYALSDHLLVWLNQSGNSFDAPFRISLPEGVRYDNTCSLQVADIQGLGISSLVLSVPHPTPRHWVCHLTTEKPWLLDGMNNNMGARHTLCYRSSAQFWLDEKAAATADRPAPACYLPFALHTLSRTEVSDEITGNRLTRTIRYRHGVWDRREREFRGFGFVEVSDAEALAKQTEGMSAPAVKRSWYATGLAAVDAQLPDEFWKGDHAAFAGFTPRFTTGDGEQEAVLDTISDDTRFWLTRAIRGTLLRSELYGADGSSQAGIPYSITESRPQVRLITEAGNSPVVWPSVIENRTSHYERVSSDPQCGQQILLTSNEYGQPLRQIGISYPRRTRPDASPYPDDLPDGLFADSFDEQQQALRLTLTQSSWHTLKDISSGIWLPAVADATRSDLFVHQAAQVPPAGLTLENLLTDSALLTSPVFGGQSQIWYQDRAGQASITSPDFPPRPSFSETAALDEAQVSALSADIDQTKLEQAGYTRSAYLFARSGEESKTLWAVRQGYITFSGADHFYLPIAAQQTLLAGKTTVTYDPYDCVVLQAKDAAGAVTSATYDWRFLAPTQITDINDNLKSVTLDALGRVTSQRFSGTENGKPAGYSDDEFPLPASADAALALSAPLPVAQCIIYVPDSWMLTGEQQQPPHVITLLTDRYDSDSQQQIRQQVVFSDGFGRVLQAASRQVNGEAWQRAANGSFVAGTNDSPVLTETTFRWAVTGRTEYDNKGQAIRAYQPYFLDSWKYVRDDSARQDLYADTHYYDPVGRERQVITAKGWLRRVIHTPWFVVSEDENDTQA

SEQ ID NO: 32 - protein, 1004 aa, Pantoea agglomerans
MSAAYVLSNLSYKLENPMSTSLYSRTPSVTILDNRGLTVRGIAYQRHPDTPAVTSERITRHQYDARGFLMQSADPRLHDAGLANVSYRTNLTGSVLRSQGVDNGITVTLNDAAGRPFLAVSNISTAGDGTEDRSQAVTRTCQYEDATLPGRPLSITEQVNGGAARITERFVYAGNAVEEKALNLAGQPVSHYDTAGLTQTDSIALTGVPLSVTRRLLKDADNPDAVADWQGTDASVWNDPLDVETYTTLSTADATGAVLTTTDAKGNLQRLAYDVAGLLSGSWLTLKDGTEQVIVTSLTYSAAGQKLREEHGNGVVTTYTYEAETQRLTGIKTARPAGHTSGAKVLQDLRYTYDPVGNVLKISNDAEETRFWRNQKVAPESAYVYDSLYQLVSATGREMANAGQQGSSSSSATVPLPADSSAFTNYTRTYAYDEAGNLTQVRHTPATGSGYTTKITVSDKSNRAVLSVLTKNPSDVDALFTAGGQQKQLQPGQSLIWTPRNELLKVMPIMRDGGTDDSESYRYDGGSQRLLKVSVQKTGNSTQTQRALYLPGLELRNTTSGDTETESLQVVTAGEAGRAQVRVLHWESGTPDSVSNDQLRYSYDNLTGSSGLELDSSGNIISMEEYYPYGGTAVWTARSAVEAKYKTVRYSGKERDATGLYYYGYRYYQPWAGRWLSADPAGTADGLNLFRMVRNNPVTLKDTNGLISTGQDARKLVAEAFVHPLHMTVFERISSEENLAMSVREAGIYTISALGEGAAAKGHNILEKTIKPGSLKAVYGDNAESILAQAKRSGFVGRVGQWDASGVRGIYAHNTPGGEDLAYPVNLKNSSANELVNAWIKFKIITPYTGDYDMHDIIKISDGKGHVPMAESNEEKGVKDMINEGVAQVDPARPFTSTAMNVVRHGPQVNFVPYMWEHEHENVVRDNGYLGVVARPGPFPVAMVHKGEWTVFDNKNELFEFYKSTNTPLPEHWSQDFVERGKGNVATPRHAEILDRNSSRLRAA

SEQ ID NO: 33 - protein, 692 aa, Pantoea agglomerans
MCSVADFDRLHNIKQENIMGTSLYSKTPSVTILDNRGLTVRDIAYQRHPDTPAVTSERITRHQYDARGFLMQSADPRLHDAGLANVSYRTNLTGSVLRSQGVDNGITVTLNDAAGRPFLAVSNISTAGDGTEDRSQAVTRTCQYEDATLPGRPLSITEQVNGGAARITERFVYAGNAVEEKALNLAGQPVSHYDTAGLTQTDSIALTGVPLSVTRRLLKDADNPDAVADWQGTDASVWNDPLDVETYTTLSTADATGAVLTTTDAKGNLQRLAYDVAGLLSGSWLTLKDGTEQVIVTSLTYSAAGQKLREEHGNGVVTTYTYEAETQRLTGIKTARPAGHTSGAKVLQDLRYTYDPVGNVLKISNDAEETRFWRNQKVVPESAYVYDSLYQLVSATGREMANAGQQGSSSSSATVPLPADSSAFTNYTRNYTYDEAGNLTQVRHTPATGSGYTTKITVSDKSNRGVLSTLTENPSDVDALFTAGGQQKQLQPGQSLIWTPRNELLKVTPVARDGGADDSESYRYDGGSLRLLKVSVQKTGNSTQTQRALYLPGLELRNTTSGDTETESLQVVTVGEAGRAQVRVLHWESGTPDSVSNDPVRYSYDNLTGSSGLELDSSGNIISMEEYYPYGGTAVWTARSAVEAKYKTVRYSGKERDATGLYYYGYRYYQPWAERGPGGHGGRAEPVQNGAQ

SEQ ID NO: 34 - protein, 2518 aa, Pantoea agglomerans
MYLTEEILAKLNAGNGKLQSTVEQIITLPDIMLHSFAQVKELAGDKLSWGEKNFLYQQAQKQLKENKMAESRILSRANPQLANAVRMGIRQSAMLGSYDDLFPQRASRFVKPGAVASMFSPAGYLTELYREARGLHDDTSDYHLDTRRPDLASMVLSQSNMDTELSTLSLSNELLLKLIQSKESLNYDQVIEKLATYRLTGTTPYNQPYETIRQAILLQDPEFNAFSNNPAVAVKINTSGLLGITSDIAPELHAILTEEITEKKTEALIKKNFGDANINQFQNLAWLAHWYGLSYEELNNLVGMIWSRDDLDPAVEHYKNSSLVTLVAEDGGSLNAVLIKRTKGHDSDDMHYAELIPVGGDKFQYNFSLIDAESSSVYYQFGTKGKNSQDLVPVIHEPLLGNTPYAVTFTLTQEQLSNPVEISLTHGSGGGDRLTSTIFTVTTYPFDTFLLKLNKLIRLYKATGISPASIRTVIESDNTDLIITESVLNQLFWTNYYTQTFEMEFSAALVLAGADIGQIAHSESQPSAFTRLFNTPLLDNQQFSASDESLDLEPGKGADAFRIAVLKRALQVNDAGLYTLYGLSFTDKDKNGKLIPFTTNIENLSALYRTRLLADIFNISVTELSMLLSVSPYASQKVDSLKGQALYQFVATLSDYMQRLKAMNWSVSDLYLMLTNSYSTVLSPEIKNLMTTLKNGLSEQDFNNTDEIAQLNATAPLIAAAMQLDFTETAAALLEWLNQLQPAGLTVAGFLSLVNQTTLEDKDVVKLVSFCQVMGQLALIVRKAALGSSEITFAVAHPAIFKKDANSLAQDIGTLFDLTQLHAFLTDCGTYASEILTSLNEGNLDVSTVATALTLDKTSLAQALAQVSESQAFSNWHELRDALQWTDAASIFNITPVALTAMVNLKFSGDNSSPYQEWVTVSKAMQVGLNQTQSAQLQASLDESLSAALSAYVIKNITPPSVTDRDELYGWLLIDNQVSAQIKTTRIAEAIASVQLYVNRSLTGQEDGVDSKVKSGQFFTADWDTYNKRYSTWAGVSELVYYPENYVDPTLRIGQTGMMDEMLQTLSQSQINLDTVSDGMGRYLTDFEEIANLKFLSGYHDNVSGRQGKTWFIGGSQSEPQKFYWRSLDYSKGDGEEFAANAWSEWNHISCAITPLPGFVRVVLFNSRLYLACVEKKEIRDSENKNKASYQLKIAHILYNGEWSAPFSHDITDLYEAGFDPSTTVMHLSVHDESDAIVCIFNNSALESDKNKGVAVNADMSFNNIDSKRVDQIISLLVPDRFIDEGNVIDNLVSELKGSEVTENKKTLENDSFTIDGSINLNKHSIDITGKANLDIQASIAVRSKASPTSHERELIGWLDESQFDYIRLFRGGYNFGQNDGILESCMISAVNSAYTCFLLRADHFSGLFSYGYDLFVFNGDGSKTYTPQVLFEDDIQGTMVLKIVLLNEDKNSKLENFESLGLMKTSAGDHQGEIVCELAKRRTPEPYCVELSRYLPSNVTVTVTSPSGNFTAKDYVLPLPAFNNGDADYKFAPFPLSLESIWGDGKSTSRDIKFTISVKDTCGKVATSELIFTLYKNTSPELITLKTSDAGAQYMQQGVYRTRLNTLFAQKLIKRVSAGIDAVLSWETQQLQEPKLGTGSYISVLIPAYIKLEHGDSRQANLQFSNVDQTGPDNGNYILWSGSLNDTPQQVTIFVPTMQTIGELQFPYDRTSGLNLSLACAAGVYLQGTFKNISASDLSLTEFVAAKNNDSKRDVEVTVLTSINTEPMDFKGANALYFWEMFYYLPMMVFKRLLSESRFTEATQWIRYVWNPDGYLVNDTPATYQWNVRPLEDETSWHANPLDSVDPDAIAQADPLHYKVATFMAYLDLLIARGDAAYRQLERDALSEAKMWYVQALDTLGDEPYLSQNTGWASPCLTDAADETTHKNRQQAMLTVRQKVASSELRTANSLTALFLPQQNAKLAGYWQTLNQRLYNLRNNLSIDGNPLSLSIYATPTDPAALLSSAVISSQGGSDLPAAVMPLYRFPVILESARSMVNQLTQFGSTLLGITERQDAEALSDLLQTQGAGLALQSIALQNSTISEIDADRAALRESLSGAQSRLNSYTTLYDENVNAGETHAMNLFLSSAILADGGQAYHTAAGALDLAPNIFGLADGGSRWGAAFTAMAGIADLAASATHTAADRISQSEAYRRRRQEWEIQRNAAQFEVSQINAQLDALAVRRESAVLQKTYLETQQGQMQAQMTFLQNKFTSKALYNWLRGKLAAIYYQFYDLTVSRCLMAEAAYSWEMKGSQDTGTFIRPGAWQGTYAGLMAGETLMLNLAQMENSYLTKEERQKEVTRTVCLSEVYAGLSSGSFALADTVTTLVGSGKGTAGTNDNGVKIDGKQLLATLKLSDLNIKTDYPESLDKAKRIKQISVTLPMLVGPYQDVRAVLSYGGSVVLPRGCTAVAVSHGMNDSGQFQLDFNDSRWLPFEGIPVDDSGTLTLSFPDITDKQQENLLLSLSDIILHIRYTIAS

SEQ ID NO: 35 - protein, 1421 aa, Pantoea agglomerans; identical to SEQ ID NO: 28

SEQ ID NO: 36 - protein, 950 aa, Pantoea agglomerans
SAAYVLSNLSYKLENPMSTSLYSRTPSVTILDNRGLSVRDIAYQRHPDTPAVTSERITRHQYDARGFLMQSADPRLHDAGLANVSYRTNLTGSVLRSQGVDNGITVTLNDAAGRPFLAVSNISTAGDGTEDRSQAVTRTCQYEDATLPGRPLSITEQVNGGAARITERFVYAGNAVEEKALNLAGQPVSHYDTAGLTQTDSIALTGVPLSVTRRLLKDADNPDAVADWQGTDASVWNDPLDVETYTTLSTADATGAVLTTTDAKGNLQRLAYDVAGLLSGSWLTLKDGTEQVIVTSLTYSAAGQKLREEHGNGVVTTYTYEAETQRLTGIKTARPAGHASGAKVLQDLRYTYDPVGNVLKISNDAEETRFWRNQKVAPESAYVYDSLYQLVSATGREMANAGQQGSSSSSATVPLPADSSAFTNYTRNYTYDEAGNLTQVRHTPATGSGYTTKITVSDKSNRGVLSTLTENPSDVDALFTAGGQQKQLQPGQSLIWTPRNELLKVTPVARDGGADDSESYRYDGGSLRLLKVSVQKTGNSTQTQRALYLPGLELRNTTSGDTETESLQVVTVGEAGRAQVRVLHWESGTPDSVSNDPVRYSYDNLTGSSGLELDSSGNIISMEEYYPYGGTAVWTARSAVEAEYKTVRYSGKERDATGLYYYGYRYYQPWAGRWLSADPAGTVDGLNLFRMVRNNPVTLVDDNGLFTSSPLLGIYEKEMKTFDSIKLSIGSYKYKPSKFDEKKGKYVSSDKYKLIMADDNDLNGYLFDEREMTSHLKDYADKFSKISRLNIGDERMKTNINFGTRISRYLLSSAQASSRENREVDVLSFERKFFAVVKKKDKSHYFGRKIYAIGEAHVLTDFEEKKRTIAIKTLVAHPYTQINESIKNRINDFDKEYNVKGIGTFATFKATNKLIGGIKGALKYKTKVLTQAVNVRSAAIAIKYGAKHVP

SEQ ID NO: 37 - DNA, 1537 bp, Pantoea agglomerans
  source 1..1537: mol_type = genomic DNA
attgaagagtttgatcatggctcagattgaacgctggcggcaggcctaacacatgcaagtcggacggtagcacagagagcttgctcttgggtgacgagtggcggacgggtgagtaatgtctgggaaactgcccgatggagggggataactactggaaacggtagctaataccgcataacgtcgcaagaccaaagtgggggaccttcgggcctcacaccatcggatgtgcccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttagggttgtaaagtactttcagcggggaggaaggcggtgcggttaataaccgtgccgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagcgaactttccagagatggattggtgccttcgggaacgctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 38 - DNA, 1537 bp, Pantoea agglomerans; identical to SEQ ID NO: 37
  source 1..1537: mol_type = genomic DNA

SEQ ID NO: 39 - DNA, 1063 bp, Pantoea agglomerans
  source 1..1063: mol_type = genomic DNA
ttgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagcgaactttccagagatggattggtgccttcgggaacgctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt